(19) United States
(12) Patent Application Publication
(10) Pub. No.: US 2025/0354978 A1
(43) Pub. Date: Nov. 20, 2025

(54) METHOD TO ASSESS POTENCY OF VIRAL VECTOR PARTICLES

(71) Applicant: Juno Therapeutics, Inc., Seattle, WA (US)

(72) Inventors: Edith NALBANDIAN, Seattle, WA (US); Rupesh AMIN, Seattle, WA (US); Neil HAIG, Seattle, WA (US); Stanislaw MORKOWSKI, Seattle, WA (US); Cyr Clovis Chua DE IMUS, Seattle, WA (US)

(73) Assignee: Juno Therapeutics, Inc., Seattle, WA (US)

(21) Appl. No.: 18/283,435

(22) PCT Filed: Mar. 21, 2022
(86) PCT No.: PCT/US2022/021226

§ 371 Date: May 13, 2024

Related U.S. Application Data
(60) Provisional application No. 63/164,532, filed on Mar. 22, 2021.

(51) Int. Cl. G01N 33/50; C12N 15/86

(52) U.S. Cl. CPC G01N 33/505; C12N 15/86; C12N 2740/15043

(57) ABSTRACT

Provided herein are cells, methods, kits and articles of manufacture, including those related to assessing the potency of viral vectors. The present disclosure relates to a method for screening for potency of a viral vector, including vectors which encode recombinant receptors that contain an extracellular antigen-binding domain and an intracellular signaling domain, such as a chimeric antigen receptor (CAR). The methods include assessing potency of a viral vector based on a detectable or measurable expression or activity of a reporter molecule(s) that are responsive to a signal through the intracellular signaling region of the T cell receptor e.g., recombinant receptor.

### CROSS-REFERENCE TO RELATED APPLICATIONS

This application is a national stage application under 35 U.S.C. § 371 of International Application No. PCT/US2022/021226, filed internationally on Mar. 21, 2022 which claims priority to U.S. provisional application 63/164,532 filed Mar. 22, 2021, the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042023200SubSeqList.txt, created Oct. 31, 2024, which is 57,930 bytes in size. The information in the electronic format of the Sequence Listing is incorporated by reference in its entirety.

### FIELD

The present disclosure relates to a method for screening for one or more potency of a viral vector, including vectors which encode recombinant receptors that contain an extracellular target-binding domain and an intracellular signaling domain, such as a chimeric antigen receptor (CAR). The methods include assessing or determining potency of a viral vector based on a detectable or measurable expression or activity of a reporter molecule, such a reporter enzyme, that is responsive to a signal through the intracellular signaling region of the T cell receptor e.g., recombinant receptor. In some embodiments, the methods can be used to screen a plurality of viral vectors, each containing a nucleic acid molecule encoding a candidate recombinant receptor, e.g. CAR, and assessing such vectors or plurality of vectors for potency. The methods can be high-throughput. Also provided are reporter cells, such as reporter T cells, cell compositions, and kits for use in the methods.

### BACKGROUND

Improved strategies are needed to determine vector potency, wherein current methods are cost prohibitive, imprecise, and not easily reproducible. Defects in current protocols for measuring vector effectiveness result in significant unwanted variation between lots of transduced cells, including in connection with adoptive immunotherapy, for use in treating cancer, infectious diseases, and autoimmune diseases. Provided are methods and cells, for use in the methods that meet such needs.

### SUMMARY

Provided herein is a method for determining potency of viral vectors, comprising a) introducing a titrated amount of a test viral vector encoding a recombinant receptor into a plurality of populations of reporter T cells, wherein each population of reporter T cells is the same and each is introduced with a different amount of the titrated test viral vector, wherein each of the reporter T cell populations comprise reporter T cells comprising a nucleic acid sequence encoding a reporter molecule operably linked to a transcriptional regulatory element of a T cell transcription factor; the recombinant receptor comprises an extracellular binding domain specific to a target, a transmembrane domain and comprises or is complexed with an intracellular signaling region comprising an ITAM-containing domain; b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule; c) measuring each of the plurality of populations of reporter T cells for the detectable signal from the reporter molecule; and d) determining, based on the measured detectable signal, the titrated amount of the test viral vector that results in a specified (e.g., half-maximal) detectable signal. In some embodiments, the target is an antigen of the recombinant receptor.

In some embodiments, provided herein is a method for determining potency of viral vectors, comprising a) introducing a titrated amount of a test viral vector encoding a recombinant receptor into a plurality of populations of reporter T cells, wherein each population of reporter T cells is the same and each is introduced with a different amount of the titrated test viral vector, wherein each of the reporter T cell populations comprise reporter T cells comprising a nucleic acid sequence encoding a reporter molecule operably linked to a transcriptional regulatory element of a T cell transcription factor; the recombinant receptor comprises an extracellular binding domain specific to an antigen, a transmembrane domain and comprises or is complexed with an intracellular signaling region comprising an ITAM-containing domain; b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule; c) measuring each of the plurality of populations of reporter T cells for the detectable signal from the reporter molecule; and d) determining, based on the measured detectable signal, the titrated amount of the test viral vector that results in a specified (e.g., half-maximal) detectable signal.

In some of any of the provided embodiments, the potency is a relative potency and the method further comprises comparing the specified (e.g., half-maximal) detectable signal of the test viral vector to a specified (e.g., half-maximal) detectable signal of a reference viral vector standard in the same assay.

Also provided herein is a method for determining potency of viral vectors, comprising a) introducing a titrated amount of a test viral vector encoding a recombinant receptor into a plurality of populations of reporter T cells, wherein each population of reporter T cells is the same and each is introduced with a different amount of the titrated test viral vector wherein each of the reporter T cell populations comprise reporter T cells comprising a nucleic acid sequence encoding a reporter molecule operably linked to a transcriptional regulatory element of a T cell transcription factor; the recombinant receptor comprises an extracellular binding domain specific to a target, a transmembrane domain and an intracellular signaling region comprising an ITAM-containing domain; b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule; c) measuring each of the plurality of populations of reporter T cells for the detectable signal from the reporter molecule; and d) determining, based on the measured detectable signal, the relative potency of the test viral vector by comparing a specified (e.g., half-maximal) detectable signal of the test viral vector to a specified (e.g., half-maximal) detectable signal of a reference viral vector standard in the same assay. In some embodiments, the target is an antigen of the recombinant receptor.

Also provided herein is a method for determining potency of viral vectors, comprising a) introducing a titrated amount of a test viral vector encoding a recombinant receptor into a plurality of populations of reporter T cells, wherein each population of reporter T cells is the same and each is introduced with a different amount of the titrated test viral vector wherein each of the reporter T cell populations comprise reporter T cells comprising a nucleic acid sequence encoding a reporter molecule operably linked to a transcriptional regulatory element of a T cell transcription factor; the recombinant receptor comprises an extracellular binding domain specific to an antigen, a transmembrane domain and an intracellular signaling region comprising an ITAM-containing domain; b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule; c) measuring each of the plurality of populations of reporter T cells for the detectable signal from the reporter molecule; and d) determining, based on the measured detectable signal, the relative potency of the test viral vector by comparing a specified (e.g., half-maximal) detectable signal of the test viral vector to a specified (e.g., half-maximal) detectable signal of a reference viral vector standard in the same assay.

In some of any of the provided embodiments, the relative potency is a percentage of the detectable signal of the test viral vector to the reference viral vector standard. In some of any of the provided embodiments, the relative potency is a ratio of the detectable signal of the test viral vector to the reference viral vector standard. In some of any of the provided embodiments, the titrated amount of a test viral vector is a serial dilution of the viral vector. In some of any of the provided embodiments, the serial dilution of the viral vector is a serial dilution based on the vector volume. In some of any of the provided embodiments, the serial dilution is a serial dilution based on the vector titer. In some of any of the provided embodiments, the viral vector titer is a functional titer, optionally wherein the functional titer is quantified by in vitro plaque assay. In some of any of the provided embodiments, the viral vector titer is a physical titer, optionally wherein the physical titer is quantified via DNA or RNA quantification by a PCR method. In some of any of the provided embodiments, the viral vector titer is quantified as Infectious Units (IU) per unit of viral vector volume. In some of any of the provided embodiments, the serial dilution is a serial dilution based on the multiplicity of infection (MOI) of the viral vector. In some of any of the provided embodiments, the MOI is quantified via viral vector titer, optionally a functional titer, per number of permissive cells in culture conditions suitable for infection.

In some of any of the provided embodiments, the amount of a test viral vector is a ratio of viral vector concentration to the number of cells in a population of reporter T cells. In some of any of the provided embodiments, the titrated amount of a test viral vector is a ratio of a constant amount of viral vector concentration to the number of cells in a the population of reporter T cells. In some of any of the provided embodiments, the amount of the test viral vector is a volume of the test viral vector. In some of any of the provided embodiments, the amount of the test viral vector is a titer of the test viral vector. In some of any of the provided embodiments, the amount of the test viral vector is a MOI of the test viral vector. In some of any of the provided embodiments, the MOI is between about 0.001 and 10 particles/cell, optionally at or about 0.01, at or about 0.1, at or about 1.0, or at or about 10 particles/cell or any value between any of the foregoing.

In some of any of the provided embodiments, the reporter T cell is an immortalized cell line. In some of any of the provided embodiments, the reporter T cell is a Jurkat cell line or a derivative thereof. In some of any of the provided embodiments, the Jurkat cell line or derivative thereof is Jurkat cell clone E6-1.

In some of any of the provided embodiments, the regulatory element comprises a response element or elements recognized by the transcription factor that is activated upon signaling through the ITAM-containing domain of the recombinant receptor induced by the recombinant receptor stimulating agent. In some of any of the provided embodiments, the T cell transcription factor is selected from the group consisting of Nur77, NF-κB, NFAT or AP1. In some of any of the provided embodiments, the T cell transcription factor is Nur77.

In some of any of the provided embodiments, the transcriptional regulatory element comprises the Nur77 promoter or portion thereof containing a response element or elements recognized by a transcription factor. In some of any of the provided embodiments, the transcriptional regulatory element is a transcriptional regulatory element within an endogenous Nur77 locus in the T cell. In some of any of the provided embodiments, the nucleic acid sequence encoding the reporter molecule is integrated in the genome of the reporter T cell at or near the endogenous locus encoding Nur77, wherein the reporter molecule is operably linked to a transcriptional regulatory element of the endogenous Nur77 locus. In some of any of the provided embodiments, the nucleic acid sequence encoding the reporter molecule is integrated by a) inducing a genetic disruption at one or more target site(s) at or near the endogenous locus encoding Nur77; and b) introducing a template polynucleotide comprising a nucleic acid encoding the reporter molecule for knock-in of the reporter molecule in the endogenous locus by homology directed repair (HDR).

In some of any of the provided embodiments, the genetic disruption is induced by a CRISPR-Cas9 combination that specifically binds to, recognizes, or hybridizes to the target site. In some of any of the provided embodiments, the RNA-guided nuclease comprises a guide RNA (gRNA) having a targeting domain that is complementary to the target site. In some of any of the provided embodiments, the nucleic acid encoding the reporter is present within the genome at a site that is at or near the final exon of the endogenous locus encoding Nur77. In some of any of the provided embodiments, the one or more target site(s) comprise, and/or the nucleic acid is present within the genome at a site comprising, the nucleic acid sequence

(SEQ ID NO: 3)
TCATTGACAAGATCTTCATG
and/or
(SEQ ID NO: 4)
GCCTGGGAACACGTGTGCA.

In some of any of the provided embodiments, the reporter molecule is or comprises a luciferase, a β-galactosidase, a chloramphenicol acetyltransferase (CAT), a β-glucuronidase (GUS), or a modified form thereof. In some of any of the provided embodiments, the reporter molecule is a luciferase, optionally firefly luciferase. In some of any of the provided embodiments, the nucleic acid sequence encoding the reporter molecule further encodes one or more marker(s) that is or comprises a transduction marker and/or a selection marker. In some of any of the provided embodiments, the transduction marker comprises a fluorescent protein, optionally eGFP.

In some of any of the provided embodiments, the reference viral vector standard is a validated viral vector lot that is representative of the same manufacturing process as the test viral vector. In some of any of the provided embodiments, the reference viral vector standard is a viral vector lot produced under good manufacturing practice (GMP). In some of any of the provided embodiments, the assessment of the reference viral vector standard is carried out in parallel with the test viral vector in the assay.

In some of any of the provided embodiments, the intracellular signaling domain is or comprises an intracellular signaling domain of a CD3 chain, or a signaling portion thereof. In some of any of the provided embodiments, the intracellular signaling domain is or comprises a CD3-zeta (CD3ζ) chain or a signaling portion thereof. In some of any of the provided embodiments, the intracellular signaling region further comprises a costimulatory signaling region. In some of any of the provided embodiments, the costimulatory signaling region comprises an intracellular signaling domain of a T cell costimulatory molecule or a signaling portion thereof. In some of any of the provided embodiments, the costimulatory signaling region comprises an intracellular signaling domain of a CD28, a 4-1BB or an ICOS or a signaling portion thereof. In some of any of the provided embodiments, the recombinant receptor is an engineered T cell receptor (eTCR). In some of any of the provided embodiments, the recombinant receptor is a chimeric antigen receptor (CAR).

In some of any of the provided embodiments, the recombinant receptor stimulating agent is a binding molecule that is or comprises a target antigen or an extracellular domain binding portion thereof, optionally a recombinant antigen, of the recombinant receptor. In some of any of the provided embodiments, the binding molecule is or comprises an extracellular domain binding portion of the antigen and the extracellular domain binding portion comprises an epitope recognized by the recombinant receptor. In some of any of the provided embodiments, the recombinant receptor stimulating agent is or comprises a binding molecule that is specific to an extracellular target binding domain of the recombinant receptor. In some of any of the provided embodiments, the recombinant receptor stimulating agent is or comprises an antibody that is specific to an extracellular target binding domain of the recombinant receptor. In some of any of the provided embodiments, the recombinant receptor stimulating agent is or comprises a binding molecule that is an anti-idiotypic antibody specific to an extracellular antigen binding domain of the recombinant receptor. In some of any of the provided embodiments, the recombinant receptor stimulating agent is or comprises a binding molecule that is an anti-idiotypic antibody specific to an extracellular antigen binding domain of the recombinant receptor.

In some of any of the provided embodiments, the recombinant receptor stimulating agent is immobilized or attached to a solid support. In some of any of the provided embodiments, the solid support is a surface of the vessel, optionally a well of microwell plate, in which the plurality of incubations are performed. In some of any of the provided embodiments, the solid support is a bead.

In some of any of the provided embodiments, the beads are from a composition having a concentration of the binding molecule of between or between about 0.5 g/mL and 500 μg/mL, inclusive, optionally at or about 5 μg/mL, 10 μg/mL, 25 μg/mL, 50 μg/mL, 100 μg/mL or 200 μg/m, or any value between the foregoing. In some of any of the provided embodiments, the beads are added at a ratio of reporter T cells to the beads that is from or from about 5:1 to 1:5, inclusive. In some of any of the provided embodiments, the beads are added at a ratio of reporter cells to the beads is from or from about 3:1 to 1:3 or 2:1 to 1:2. In some of any of the provided embodiments, the beads are added at a ratio of reporter cells to the beads that is or is about 1:1.

In some of any of the provided embodiments, the recombinant receptor stimulating agent is an target-expressing cell, optionally wherein the cell is a clone, from a cell line, or a primary cell taken from a subject. In some of any of the provided embodiments, the target-expressing cell is a cell line. In some embodiments, the target is an antigen of the recombinant receptor and thus, in some cases, the target-expressing cells are antigen-expressing cells. In some of any of the provided embodiments, the target-expressing cell is a cell that has been introduced, optionally by transduction, to express the target of the recombinant receptor. In some of any of the provided embodiments, the target-expressing cells are added at a ratio of antigen-expressing cells to the reporter T cells of from or from about 1:1 to 10:1. In some of any of the provided embodiments, the target-expressing cells are added at a ratio of target-expressing cells to the reporter T cells of from or from about 1:1 to 6:1.

In some of any of the provided embodiments, the recombinant receptor stimulating agent is an antigen-expressing cell, optionally wherein the cell is a clone, from a cell line, or a primary cell taken from a subject. In some of any of the provided embodiments, the antigen-expressing cell is a cell line. In some of any of the provided embodiments, the cell line is a tumor cell line.

In some of any of the provided embodiments, the antigen-expressing cell is a cell that has been introduced, optionally be transduction, to express the antigen of the recombinant receptor. In some of any of the provided embodiments, the antigen-expressing cells are added at a ratio of antigen-expressing cells to the reporter T cells of from or from about 1:1 to 10:1. In some of any of the provided embodiments, the antigen-expressing cells are added at a ratio of antigen-expressing cells to the reporter T cells of from or from about 1:1 to 6:1.

In some of any of the provided embodiments, the plurality of incubations are performed in a flask, a tube, or a multi-well plate. In some of any of the provided embodiments, the plurality of incubations are each performed individually in a well of a multi-well plate. In some of any of the provided embodiments, the multi-well plate is a 96-well plate, a 48-well plate, a 12-well plate or a 6-well plate.

In some of any of the provided embodiments, the detectable signal is measured using a plate reader. In some of any of the provided embodiments, the detectable signal is luciferase luminescence and the plate reader is a luminometer plate reader.

In some of any of the provided embodiments, the viral vector is an adenoviral vector, adeno-associated viral vector, or a retroviral vector. In some of any of the provided embodiments, the viral vector is a retroviral vector. In some of any of the provided embodiments, the viral vector is a lentiviral vector. In some of any of the provided embodiments, the lentiviral vector is derived from HIV-1.

In some of any of the provided embodiments, the detectable signal is luciferase luminescence.

### BRIEF DESCRIPTION OF THE DRAWINGS

FIG. 1A shows an exemplary vector potency assay wherein transduced reporter cells are incubated with antigen expressing target cells for a period of time before the luciferase substrate is added.

FIG. 1B depicts results testing expression of enhanced green fluorescent protein (EGFP) and luciferase enzymatic activity in the presence of activation agonists and substrate in several exemplary Jurkat reporter cells that were generated containing a Nur77-Luciferase-EGFP knock-in reporter. FIG. 1C shows a dose-dependent curve of luciferase activity among exemplary Jurkat reporter cells in the presence of decreasing PMA/ionomycin concentration.

FIG. 2A depicts an exemplary 3-plate assay format for a vector potency assay.

FIG. 2B depicts an exemplary dose response curve for an exemplary test sample, in which the vector volume (in microliters) is plotted on the x-axis and the Relative Light Units (RLU) on the y-axis, which is directly proportional to vector function.

FIG. 2C shows the dose response curves for the test and reference samples and the test sample's 50% effective concentration (EC50) compared to the reference standard's EC50.

FIG. 2D shows a further exemplary dose response curve for cells transduced with a CD19 targeted CAR.

FIG. 3 depicts an exemplary dose response curve for cell transduced with a BCMA targeted CAR in which the vector MOI (IU/cell) is plotted on the x-axis and the relative luminescence units on the y axis.

FIG. 4A depicts a calculated line of best fit for a potency assay as described, with the corresponding residual distribution shown in FIG. 4B.

FIG. 5 depicts the specificity of the provided potency assay, as determined by detectable signal from the reference standard but not from a non-specific vector, as determined by measuring Relative Light Units (RLU).

FIG. 6 depicts the stability-indicating specificity of the provided potency assay as determined by assessing vector potency of a viral vector after at least one forced-stress conditions. The results demonstrated a decreased vector potency, indicating the specificity of the assay as stability indicating.

FIG. 7 depicts exemplary readouts across 4 independent assays performed by separate operators.

### DETAILED DESCRIPTION

Provided herein are methods for assessing or determining relative potency of a viral vector, such as a viral vector used to transduce reporter cells (e.g., reporter cell composition). The provided embodiments relate to methods using engineered reporter cells such as those engineered to express recombinant proteins such as expressing recombinant receptors. The receptors may include chimeric receptors, e.g., chimeric antigen receptors (CARs), and other transgenic antigen receptors including transgenic T cell receptors (TCRs).

In some contexts, the provided embodiments, including the cells, methods, kits and articles of manufacture, can be adapted to assess the potency of different types of viral vectors. In some embodiments, the methods can be used to assess the potency of a plurality of viral vectors compositions, e.g., a plurality of viral vectors compositions with different properties or potencies.

In some embodiments, the methods employ a transduced reporter cell, e.g., a reporter T cell, that contains a reporter, such as a reporter enzyme, that is responsive to a signal through the intracellular signaling region of the recombinant receptor, such as a primary activation signal in a T cell, a signaling domain of a T cell receptor (TCR) component, and/or a signaling domain comprising an immunoreceptor tyrosine-based activation motif (ITAM). In some embodiments, the reporter cells, e.g., reporter T cells, have a reporter that is responsive to a signal through the intracellular signaling region of a receptor, in some embodiments a recombinant receptor. In some embodiments, the methods involve the use of such cells. In some embodiments, the reporter T cell comprises a nucleic acid sequence encoding a reporter molecule or reporter molecules operably linked to a transcriptional regulatory element of the endogenous locus encoding Nur77. In some embodiments, the reporter T cell contains a reporter molecule or molecules knocked-in at the endogenous Nur77 locus, such that the expression of the reporter or reporters is controlled by the endogenous transcriptional regulatory elements of the Nur77 gene.

Cell based therapies, including adoptive T cell therapies (such as those involving the administration of cells expressing chimeric receptors specific for a disease or a disorder of interest, such as chimeric antigen receptors (CARs) and/or other recombinant antigen receptors, as well as other adoptive immune cell and adoptive T cell therapies) can be effective in the treatment of cancer and other diseases and disorders. For cell and gene therapies, one aspect of production is the vector used to introduce the gene of interest into cells for administration to a patient or directly to the patient as a therapeutic composition. Inherent to production of viral vectors and their use in downstream therapies is the complexity of viral vectors that necessitates in-process characterization to limit lot-to-lot variability. In certain contexts, available approaches to assess the potency of such vectors may not be satisfactory in one of more aspects of cost, reproducibility, precision, or practicality within the Good Manufacturing Practice (GMP) framework.

Current techniques for measuring vector potency are inconsistent, cost prohibitive, and poorly reproducible. Unlike conventional biologics, viral vectors comprise both protein and nucleic acid components. As a result, there are many detection methods available that target either the viral genome or viral proteins. Methods of characterizing viral vectors include determining physical viral titer through means known in the art, such as DNA hybridization, Real-time PCR (qPCR, ddPCR), optical density (A260/280), NanoSight, and HPLC. In some aspects, quantitative PCR (qPCR) can be used to measure vector potency as a means of transgene expression. qPCR relies on a plasmid DNA standard curve to calculate the viral titer, which can result in variation from batch to batch. Digital droplet PCR (ddPCR) does not quantify from a standard curve, however the selection of the PCR target sequence as well the design of the primers can have a significant impact on the robustness of any PCR based strategy. An Enzyme-Linked Immunosorbent Assay (ELISA) can be used to measure viral protein present in a sample, but is dependent on the availability of appropriate serotyped antibodies. Physical titer often is subject to substantial variability as molecular assays are affected by numerous experimental factors which can directly impact the accuracy of the titer/and or potency calculations. Standards and controls for these are of critical importance, as often there is observed variability in the viral vector manufacturing between lots

In some aspects, viral vectors can also be assessed by measuring the infectious or functional titer of a virus composition. Infectious titer can be measured by a number of cell based assays known to those skilled in the art, including plaque assays, fluorescence foci assays, end point dilution assays (TCID50) or other cell based assays. Generally, these cell based assays are highly product specific as indicator or reporter cells are transfected with the viral vector, and the expression of the transgene is measured (e.g., RT-PCR, ELISA or FACS). In some aspects, functional titer is expressed as transducing units per mL (TU/mL) for lentiviral or retroviral vectors. Similarly, vector titer can also be generally expressed as plaque-forming units per mL (PFU/mL) or infectious units per mL (IFU/mL). The latter term is used for viral vectors that do not lyse cell membranes and therefore are not compatible with the standard plate based plaque assay. However functional titer usually takes significant time to determine, and is often considered not practical during intermediate or beginning stages or viral vector production.

In some aspects, viral vector potency is established in a variety of cell-based assays, but the output of the assay can vary. For instance, in some cases, viral vector potency is assessed by determining the degree or percentage of CAR expression or assessing cytokine production. In some embodiments, such assays may be long in duration and/or may be subject to high variability (e.g. 20-30% prevision). Further, many existing assays are not carried out in a relative format so day to day variability is not accounted for. This means a risk of many existing viral vector potency assays is that the results may be variable assay to assay, even from the same test viral vector.

Another important consideration for viral vector analytics is the relatively small lot sizes, which limit the availability of sufficient material for method development, assay qualification/validation and stability testing. There is much less material made during viral vector manufacturing than the manufacturing of conventional biologics, such as monoclonal antibodies (King et al. “Viral Vector Characterization: A Look at Analytical Tools” CellCultureDish.Com, October 2018). Thus, there is a need to provide a more effective method of assessing potency of a viral vector. In some aspects, the provided methods permit potency to be more easily, rapidly, and reliably determined.

Thus, in some contexts, the ability to efficiently and reliably assess the potency of a viral vector can be a useful tool for the generation of cell and gene based therapies. Improved strategies are also needed to assess the potency of a viral vectors produced from different manufacturing lots and different processes, including in a relatively fast and reliable manner. The provided methods can be used to assess release of genetic material for use in engineering of cell therapies, including T cell therapies.

The provided embodiments for assessing viral vector potency are particularly useful in connection with viral vectors used for delivering certain transgenes to T cells that encode recombinant receptors, such as CARs, containing an intracellular signaling domain of a T cell receptor (TCR) component, and/or a signaling domain comprising an immunoreceptor tyrosine-based activation motif (ITAM). The provided reporter cells contain a reporter molecule operably linked to a transcriptional regulatory element of a T cell transcription factor that is responsive to a transcription factor induced by signaling upon stimulation of such signaling domain. In some embodiments, expression of the reporter or reporters, among other parameters, can be assessed after incubation of the reporter T cells in the presence or absence of a recombinant receptor stimulating agent that binds to the binding domain of the T cell receptor and/or an agent that induces or is capable of inducing a signal through the intracellular signaling region of the receptor.

The provided embodiments, in some contexts, are based on the observation that the expression of the endogenous Nur77 gene is cell intrinsic, and/or is not substantially affected or influenced by other signaling pathways, such as cytokine signaling or toll like receptor (TLR) signaling (see, e.g., Ashouri et al., (2017) J. Immunol. 198:657-668), which may act in a cell extrinsic manner and may not depend on signaling through the recombinant receptor. In some contexts, Nur77 expression is sensitive to a primary activation signal in a T cell, signals from a signaling domain of a T cell receptor (TCR) component, and/or a signaling domain comprising an immunoreceptor tyrosine-based activation motif (ITAM). In some contexts, the response of Nur77 reporter is dose-responsive to signals through the signaling regions. Further, in some embodiments, the provided reporter T cells contain nucleic acid sequences encoding the reporter molecule or molecules knocked into the endogenous Nur77 locus, providing a stable reporter cell line that can generate consistent results, e.g., not dependent on the location of random genomic integration or copy number and/or loss of reporter. Such reporter cells can be used to screen the potency of numerous viral vectors, simultaneously with consistent readouts.

In particular embodiments, the assay is carried out with reporter cells in which the reporter molecule is an enzyme, such as luciferase. An advantage of using an enzyme-based assay, such as luminescence-based assay, is that it can output signals of several logs of range, whereas fluorescent based reporters are often not bright enough to offer such a quantitative range. Further, luminescence based detection methods also can provide high sensitivity and low background intensity. In addition, luciferase or other enzymes are more compatible in plate-based and can be measured in solution, offering the possibility of a rapid read-out. Further, due to the dose-responsiveness of the induced signal by the T cell transcription factor, particularly as provided by the Nur77 reporter system, along with the high sensitivity and wide range of detection of luminescent-based reporter, the provided methods permit a wide linear range that includes a true linear range of the potency of the viral vector. These features of the provided assays offer advantages that are not possible with existing methods for measuring viral vector potency.

The methods provided herein are designed to more comprehensively assess the relative potency of a viral vector. The methods provided herein are designed to provide a more biologically relevant measure of a viral vector potency. In some embodiments, the potency of a viral vector composition determined according to the methods described herein may provide improved measures of manufacturing control and/or variability, which in turn can allow for improved assessment of vector release for use in genetic engineering, including in connection with assessing vector stability.

In some embodiments, the methods provided herein, reduce or eliminate sources of variability. For example, the methods provided herein are robust to variability that may arise due to plate location bias, operator bias, and/or day to day sampling or testing. In some cases, eliminating variability, such as variability due to plate location bias, operator bias and/or sampling or testing, allows for comparison of viral vector lot compositions.

The methods provided herein include assay formats including a series of incubations in which different titrated ratios of viral vector are introduced into cells of the reporter cell composition for assessment of reporter signal induced by a recombinant receptor stimulating agent (e.g., binding molecule). In some embodiments, the measure of potency includes measurements of a detectable signal of the reporter molecule stimulated by binding of a recombinant receptor stimulating agent (e.g. binding molecule) to the recombinant receptor across a plurality of titrated ratios of the viral vector. The ability of the methods to assess reporter activity at different titrated ratios or viral vector allows determination, estimation, and/or extrapolation of the potency of the viral vector lot to recombinant receptor (i.e., antigen) specific stimulation. In some embodiments, the range of measurements can used to extract, estimate, and/or determine the potency of a viral vector as measured by how engineered cells of a particular reporter cell composition respond to different levels of recombinant receptor stimulation (i.e., titrated vector).

In some embodiments, the potency of a viral vector is expressed as a value or measure of the titrated ratio, and/or amount or concentration (e.g. titer) or volume of viral vector determined based on the detectable signal (e.g. luminescence) of the reporter molecule. In some embodiments, the potency of a viral vector composition is the value or measure of the titrated ratio, and/or amount or concentration or volume of viral vector at which the specified value (e.g., half-maximal value (e.g., 50% of maximum activity)) of the detectable signal (e.g. luminescence signal) occurs. In some embodiments, the potency of a viral vector composition is the titrated ratio at which the specified value (e.g., half-maximal value (e.g., 50% of maximum activity)) of the detectable signal occurs. In some embodiments, the potency of the viral vector composition is the concentration of viral vector at which the specified (e.g., half-maximal) value of the detectable signal (e.g. luminescence signal) occurs. In some embodiments, the method is a volume-based titration and the potency of the viral vector composition is the volume of a particular viral vector lot at which the specified (e.g., half-maximal) value of the recombinant receptor-dependent activity occurs. In some embodiments, the specified (e.g., half-maximal) value of the detectable signal (e.g. luminescence) reflects the titrated ratio, concentration (e.g. titer) of viral vector, and/or volume, at which a specified effective stimulation (e.g., 50% effective stimulation (ES₅₀)) of the reporter T cells occurs, according to the measured detectable signal from the reporter molecule present in the reporter cells.

In some embodiments, the potency of a viral vector is expressed as a value or measure of the titrated ratio, and/or amount or concentration (e.g. titer) or volume of viral vector determined based on the detectable signal (e.g. luminescence) of the reporter molecule. In some embodiments, the potency of a viral vector composition is the value or measure of the titrated ratio, and/or amount or concentration or volume of viral vector at which the specified (e.g., half-maximal value (e.g., 50% of maximum activity)) of the detectable signal (e.g. luminescence signal) occurs. In some embodiments, the potency of a viral vector composition is the titrated ratio at which the specified (e.g., half-maximal value (e.g., 50% of maximum activity)) of the detectable signal occurs. In some embodiments, the potency of the viral vector composition is the concentration of viral vector at which the specified (e.g., half-maximal) value of the detectable signal (e.g. luminescence signal) occurs. In some embodiments, the method is a titration based on Multiplicity of Infection (MOI) and the potency of the viral vector composition is the IU/cell ratio of a particular viral vector lot at which the specified (e.g., half-maximal) value of the recombinant receptor-dependent activity occurs. In some embodiments, the specified (e.g., half-maximal) value of the detectable signal (e.g. luminescence) reflects the titrated ratio, concentration (e.g. MOI) of viral vector, and/or IU/cell ratio, at which a specified effective stimulation (e.g., 50% effective stimulation (ES50)) of the reporter T cells occurs, according to the measured detectable signal from the reporter molecule present in the reporter cells.

In some embodiments, the potency of the viral vector composition is a relative potency. For example, the titrated ratio at which half-maximal detectable signal is measured for a viral vector can be compared to the titrated ratio at which half-maximal detectable signal is measured for a reference standard or for a control viral vector. It should be appreciated that concentration or amount or volume or MOI of viral vector may be used in place of the titrated ratio, if applicable. In some embodiments, the reference standard or control is a viral vector having a known and/or validated titrated ratio at which the specified (e.g., half-maximal) detectable signal occurs in the assay. In some embodiments, the reference standard or control is a commercially available viral vector for which a titrated ratio at which the specified (e.g., half-maximal) detectable signal has been determined, for example using a method as described herein. In some embodiments, the reference standard or control is a different viral vector for which a titrated ratio at which the specified (e.g., half-maximal) detectable signal has been determined, for example using a method as described herein. In some embodiments, the different viral vector composition contains nucleic acid encoding the same recombinant receptor that binds to the same target as the test viral vector. In some embodiments, the reference viral vector standard is one that has been manufactured from a process determined to be representative of the manufacturing process of the test viral vector. In some embodiments, the reference viral vector standard is GMP (Good Manufacturing Practice) grade. In some embodiments, the relative potency is a ratio determined by dividing the titrated ratio that results in the specified (e.g., half-maximal) value of the test viral vector by the titrated ratio that results in the specified (e.g., half-maximal) value of the reference standard or control. In some embodiments, the relative potency is a percentage determined by dividing the titrated ratio that results in the specified (e.g., half-maximal) value of the test viral vector composition by the titrated ratio that results in the specified (e.g., half-maximal) value of the reference standard and multiplying by 100.

The methods, including assays, provided herein for assessing potency of a viral vector composition allows for different viral vector compositions, including references standards, to be compared. The ability to compare viral vector compositions provides a method not only for identifying viral vector compositions with improved, optimal, and/or consistent potencies, but also to: identify candidate viral vector compositions for further development and/or analysis; identify manufacturing processes and procedures that yield viral vector compositions with improved or optimal potency; identify manufacturing procedures or processes that yield viral vector compositions with consistent potency, and/or estimate a variability inherent to a manufacturing procedure. In particular embodiments, the methods can be used in a release assay to confirm a viral vector genetic material is suitable for use in connection with methods of engineering cell therapies with a recombinant receptor (e.g. a CAR).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS FOR ASSESSING THE POTENCY OF VIRAL VECTORS

Provided herein are methods of assessing potency of a test viral vector, such as a viral vector encoding a recombinant receptor (e.g., CAR). Provided herein is a reporter T cell composition containing T cells (e.g., CD3+, CD4+, CD8+ T cells) transfected using a test viral vector to express a recombinant receptor (e.g., CAR), wherein the potency of the test viral vector is measured using an assay including a plurality of incubations, where each of the plurality of incubations includes culturing cells of the reporter cell composition containing cells engineered to express a recombinant receptor with a recombinant receptor stimulating agent, for example an antigen, antigen-expressing cell, or other binding domain able to bind to the recombinant receptor, and where binding of the recombinant receptor stimulating agent to the recombinant receptor stimulates a detectable signal in the reporter cell. In some embodiments, the detectable signal is enzymatic, such as the expression of an enzyme which converts available substrate into a detectable product, i.e., a luciferase reaction.

The methods provided herein for determining potency may be performed with replication. For example, an assay may be performed 2, 3, 4, 5, or more times. In some embodiments, replicates are used to confirm accuracy and/or precision of the assay, including the consistency of measured of detectable signal and/or determined potency and/or relative potency of a test viral vector. In some embodiments, a single assay is conducted by performing the assay on a particular test viral vector in duplicate or triplicate. In some embodiments, the assay is performed in duplicate. In some embodiments, the assay is performed in triplicates. In some cases where the assay is performed, for example, in duplicate or triplicate, the measured detectable signal from each of the replicates is used to provide a statistical measure of the measured detectable signal. For example, in some cases, an average, median, standard deviation, and/or variance of each measure of the detectable signal is determined. In some embodiments, an average of each measure of the detectable signal is determined. In some embodiments, a standard deviation of each measure of the detectable signal is determined. In some embodiments, the average measure of detectable signal are fit using a mathematical model to produce a curve of the detectable signal. In some embodiments, the curve is normalized to the average maximal value. In some embodiments, the average titrated ratio that results in half-maximal detectable signal in the assay is the potency of the test viral vector. In some embodiments, the potency of the test viral vector is a relative potency determined by taking an average titrated ratio that results in half-maximal detectable signal in the assay and comparing the average titrated ratio to a single or average titrated ratio that results in half-maximal detectable signal in a reference viral vector. In some embodiments, the relative potency is the average potency of the test viral vector divided by the single or average potency of the reference viral vector. In some embodiments, the relative potency is expressed as a ratio. In some embodiments, the relative potency is expressed as a percentage.

a. Introducing Titrated Viral Vector into Reporter Cells

In some embodiments, a plurality of populations of reporter T cells are generated in which a constant number of cells of the reporter composition are introduced, such as transduced, with differing or titrated amounts of test viral vector to generate a plurality of different titrated ratios. In some embodiments, each of the plurality of a plurality of populations of reporter T cells contains a different titrated amount of viral vector, such as a different ratio, concentration or volume or MOI of the test viral vector. In some embodiments, each of the plurality of populations of reporter T cells is generated by introducing a constant number of cells of the reporter cells with a differing amount, concentration, MOI, or volume of test viral vector to generate a plurality of different titrated ratios.

In some embodiments, the titrated amount of a test viral vector is a serial dilution of a the viral vector. For instance, a range of serially diluted amounts (e.g. volumes, titers or MOI) of the viral vector are assessed among each of the plurality of populations of reporter cells. In some embodiments, the serial dilution of the viral vector is a serial dilution based on the vector volume, In some embodiments, the serial dilution is a serial dilution based on the viral vector titer.

In some embodiments, the titrated amount is a ratio of a constant amount of the test viral vector to the number of cells in each of the plurality of populations of reporter cells.

Methods of characterizing viral vectors include determining physical viral titer, such as by any of a variety of known methods such as by DNA hybridization, or PCR methods such as Real-time PCR (qPCR, ddPCR), optical density (A260/280), NanoSight, and HPLC. In some embodiments, physical titer may be done by quantitation of viral RNA or DNA by a PCR method. In some aspects, quantitative PCR (qPCR) can be used to measure vector potency as a means of transgene expression. qPCR relies on a plasmid DNA standard curve to calculate the viral titer, which can result in variation from batch to batch. Digital droplet PCR (ddPCR) does not quantify from a standard curve, however the selection of the PCR target sequence as well the design of the primers can have a significant impact on the robustness of any PCR based strategy. An Enzyme-Linked Immunosorbent Assay (ELISA) can be used to measure viral protein present in a sample, but is dependent on the availability of appropriate serotyped antibodies. Physical titer often is subject to substantial variability as molecular assays are affected by numerous experimental factors which can directly impact the accuracy of the titer/and or potency calculations. Standards and controls for these are of critical importance, as often there is observed variability in the viral vector manufacturing between lots. In some embodiments, the viral vector titer is a physical titer.

In some aspects, viral vectors can also be assessed by measuring the infectious or functional titer of a virus composition. Infectious titer can be measured by a number of cell based assays known to those skilled in the art, including plaque assays, fluorescence foci assays, end point dilution assays (TCID50) or other cell based assays. Generally, these cell based assays are highly product specific as indicator or reporter cells are transfected with the viral vector, and the expression of the transgene is measured (e.g., RT-PCR, ELISA or FACS). In some aspects, functional titer is expressed as transducing units per mL (TU/mL) for lentiviral or retroviral vectors. Similarly, vector titer can also be generally expressed as plaque-forming units per mL (PFU/mL) or infectious units per mL (IU/mL). The latter term is used for viral vectors that do not lyse cell membranes and therefore are not compatible with the standard plate based plaque assay. However functional titer usually takes significant time to determine, and is often considered not practical during intermediate or beginning stages or viral vector production. In some embodiments, the viral vector titer is a functional titer. In some embodiments, the viral vector titer is quantified in IU/mL.

In some embodiments, the serial dilution of the viral vector is a serial dilution based on the Multiplicity of Infection (MOI) of the viral vector, In some embodiments, the serial dilution is a serial dilution based on the viral vector titer. In some aspects, MOI of a viral vector can be determined as the ratio of viral vector particles to cells present in a population (e.g., the ratio of test viral vector particles to cells in a population of permissive cells). Quantification of viral vector particles can, in some aspects, be quantified via titer. In some embodiments, MOI is quantified using a functional titer. In some aspects. Functional titer can be determined using the methods described above, including a plaque assay or other in vitro infection assays known in the art.

In some embodiments, at or at least 3, 4, 5, 6, 7, 8, 9, 10, 11, 12, 13, 14, 15, or more incubations are performed, each incubation containing a different ratio of test viral vector to reporter cells, i.e. a titrated amount that is a volume or IU/cell ratio. In some embodiments, at or at least 3 series of titrations are performed, each containing introduction of a different serial dilution of test viral vector to the reporter cells. In some embodiments, at or at least 6 series of titrations are performed, each containing introduction of a different serial dilution of test viral vector to the reporter cells. In some embodiments, at or at least 10 serial dilutions are performed, each containing a different serial dilution of test viral vector to the reporter cells.

In some embodiments, the methods for determining potency of viral vectors, includes a) introducing (e.g. transducing) a titrated amount of a test viral vector encoding a recombinant receptor into a plurality of populations of reporter T cells, wherein each population of reporter T cells is the same and each is introduced (e.g. transduced) with a different amount of the titrated test viral vector, wherein: each of the reporter T cell populations comprise reporter T cells comprising a nucleic acid sequence encoding a reporter molecule operably linked to a transcriptional regulatory element of a T cell transcription factor, and in which the recombinant receptor comprises an extracellular binding domain specific to an antigen, a transmembrane domain and comprises or is complexed with an intracellular signaling region comprising an ITAM-containing domain. In provided embodiments, the methods further include incubating each of the plurality of populations of reporter T cells in the presence of a recombinant receptor stimulating agent, wherein binding of the recombinant receptor stimulating agent to the recombinant receptor induces signaling through the intracellular signaling region of the recombinant producer to produce a detectable signal from the reporter molecule. In the provided methods, the methods include measuring each of the plurality of populations of reporter T cells for the detectable signal from the reporter molecule, and then determining, based on the measured detectable signal, the titrated amount of the test viral vector that results in a half-maximal detectable signal.

In some embodiments, the titrated amount that results in half-maximal detectable signal is compared to a titrated ratio that results in half-maximal detectable signal in a reference standard, such as a reference viral vector. For example, the titrated ratio of the test viral vector that results in half-maximal detectable signal is divided by a titrated ratio that results in half-maximal detectable signal in a reference viral vector, for example determined according to the methods described herein, to yield a relative potency. In some embodiments, the relative potency is expressed as a ratio. In some embodiments, the relative potency is expressed as a percentage.

In some embodiments, provided herein is a method for determining potency of viral vectors, that includes introducing (e.g. transducing) a titrated amount of a test viral vector encoding a recombinant receptor into a plurality of populations of reporter T cells, wherein each population of reporter T cells is the same and each is introduced (e.g. transduced) with a different amount of the titrated test viral vector, wherein each of the reporter T cell populations comprise reporter T cells comprising a nucleic acid sequence encoding a reporter molecule operably linked to a transcriptional regulatory element of a T cell transcription factor, and in which the recombinant receptor comprises an extracellular binding domain specific to an antigen, a transmembrane domain and an intracellular signaling region comprising an ITAM-containing domain. In provided methods, the methods further include incubating each of the plurality of populations of reporter T cells in the presence of a recombinant receptor stimulating agent, wherein binding of the recombinant receptor stimulating agent to the recombinant receptor induces signaling through the intracellular signaling region of the recombinant producer to produce a detectable signal from the reporter molecule. In the provided methods, the methods further include measuring each of the plurality of populations of reporter T cells for the detectable signal from the reporter molecule; and determining, based on the measured detectable signal, the relative potency of the viral test viral vector by comparing the half-maximal detectable signal to a half-maximal detectable signal of a reference viral vector standard in the same assay.

The assay provided herein may be performed in any vessel(s) suitable for a plurality of incubations. In some embodiments, the assay is performed in multiwell plates.

The conditions under which the introduction (e.g. transduction) with the reporter cell composition and viral vector is performed can include one or more of particular media, temperature, oxygen content, carbon dioxide content, time, and/or agents, e.g., nutrients, amino acids, antibiotics, ions. The duration of the introduction of the viral vector, such as transduction, is contemplated to be commensurate with at least the minimal amount of time for introduction of the viral vector into the reporter cell (e.g., transduction as described in Section 1.A.3). In some embodiments, the introduction (e.g. transduction) is performed for at, about, or at least 24, 36, 48, 60, or 72 hours. In some embodiments, the introduction (e.g. transduction) is performed for at, about, or at least 24 or 48 hours. In some embodiments, the introduction (e.g. transduction) is performed for between at or about 24 hours and at or about 72 hours. In some embodiments, the introduction (e.g. transduction) is performed for between at or about 24 hours and at or about 48 hours.

In some embodiments, the introduction (e.g. transduction) is performed at a temperature from about 25 to about 38° C., such as from about 30 to about 37° C., for example at or about 37° C. ±2° C. In some embodiments, the introduction (e.g. transduction) is performed with a CO₂ level from about 2.5% to about 7.5%, such as from about 4% to about 6%, for example at or about 5%+0.5%. In some embodiments, the introduction (e.g. transduction) is performed at a temperature of or about 37° C. and/or at a CO₂ level of or about 5%.

### 1. Reporter Cells

Provided herein are cells, methods, vectors, polynucleotides, pluralities of cells, pluralities of polynucleotides, kits and articles of manufacture, including those related to assessing the potency of viral vectors, such as methods of assessing the activity of recombinant receptors, e.g., chimeric antigen receptors (CARs).

In some embodiments, provided are cells, such as reporter T cells, for assessing potency of the viral vector. In some embodiments, the reporter T cell comprises a reporter molecule or molecules, wherein the expression of a reporter molecule or molecules is responsive to a signal through the intracellular signaling region of the T cell receptor. In some embodiments, the provided cells include reporter T cells. In some embodiments, the reporter T cells contains nucleic acid sequences encoding a reporter molecule operably linked to a transcriptional regulatory element or a variant thereof of a Nur77, wherein the transcriptional regulatory element optionally is a transcriptional regulatory element within an endogenous Nur77 locus in the T cell. In some aspects the provided cells such as provided reporter T cells contain nucleic acid sequence encoding a reporter molecule or molecules operably linked to a transcriptional regulatory element, such as a transcriptional regulatory element of the endogenous locus encoding Nur77. In some embodiments, the provided cells can be used to assess activity of one or more viral vectors, e.g., for screening a plurality or a library of vector encoded candidate receptors.

Provided embodiments also include methods of assessing transduction efficiency of viral vectors such as those using any of the provided cells or constructs. In some embodiments, the vector contains a nucleic acid encoding a recombinant receptor. In some embodiments, the recombinant receptor is a CAR. In some embodiments, the methods involve incubating one or more reporter T cells, such as T cells each comprising i) a recombinant receptor, such as a recombinant receptor that is a CAR comprising an intracellular signaling region and ii) a reporter molecule or molecules, wherein the expression of said reporter molecule(s) is responsive to a signal through the intracellular signaling region of the recombinant receptor, wherein the incubating is carried out in the presence and/or absence of an agent that binds to the binding domain of the recombinant receptor and/or an agent that induces or is capable of inducing a signal through the intracellular signaling region of the recombinant receptor; and assessing the one or more reporter T cells for expression or activity of the reporter molecule(s). In some embodiments, the methods can employ any of the cells, e.g., reporter T cells, described herein.

In some embodiments, also provided are pluralities (and/or libraries) of reporter T cells that include one or more of any of the reporter T cells generated by the methods described herein.

In some embodiments, also provided are reporter T cells, polynucleotides encoding a recombinant receptor, binding domain, or recombinant receptor identified by, or present in the cell identified by any of the methods provided herein.

Provided herein are cells, such as T cell lines, that contain a reporter molecule or molecules that are capable of being expressed upon signal through the intracellular signaling region of the T cell receptor, including a recombinant receptor. Also provided are methods of using such cells, e.g., methods of assessing potency of viral vectors using such cells. In some embodiments, the methods provided herein include assessing potency, e.g., transduction efficiency, of a vector encoding a recombinant receptor, e.g., CAR, in a T cell. In some embodiments of the methods provided herein, the potency is assessed in T cells, such as a T cell line. In some embodiments, the T cell comprises a reporter molecule or molecules, e.g., reporter molecules that are capable of being expressed upon signal through the intracellular signaling region of the T cell receptor and/or binding and/or recognition of the recombinant receptor to an antigen or epitope. In some embodiments, provided are reporter T cells, such as reporter T cell lines, comprising a nucleic acid sequence encoding a reporter molecule or molecules operably linked to a transcriptional regulatory element of the endogenous locus encoding Nur77.

In some embodiments, provided are T cells, such as T cells comprising reporter molecule(s) or reporter T cells. In some embodiments of the methods provided herein, T cell, such as a reporter T cell, is employed to assess potency of a viral vector e.g. transduction efficiency. In some embodiments, the T cell is a T cell line, such as a Jurkat-derived cell line. In some embodiments, provided are reporter T cells that are derived from a T cell line. In some embodiments, provided are reporter T cells which stably express a fluorophore, such as any fluorescent protein. Examples of fluorescent proteins include green fluorescent protein (GFP), yellow fluorescent protein (YFP), cerulean/cyan fluorescent protein (CYP), or enhanced GFP (eGFP). In some embodiments, the T cell is a T cell line expressing a fluorescent protein and containing a reporter molecule, such as reporter molecules capable of producing a detectable signal or catalyzing measurable activity upon signal through the intracellular signaling region of a recombinant receptor. Also provided are compositions containing any of the cells, such as reporter T cells, described herein.

In some aspects, the T cells or T cell compositions into which the viral vectors are introduced, can be referred to as “host cells” or “host cell lines.” In some embodiments, the host cell is a T cell. The terms “host cell,” “host cell line,” and “host cell culture” are used interchangeably and refer to cells into which exogenous nucleic acid molecules have been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

In some embodiments, the cell or cell line is an immortalized cell line and/or a clonal cell line. In some embodiments, the cell or cell line is a transformed cell line. In some embodiments, the cell or cell line is a T cell line. In some embodiments, the cell or cell line is a cell line capable of transmitting, transducing, and/or mediating signaling through CD3. For example, the cell or cell line contains or expresses components of the T cell receptor (TCR) signaling pathway containing CD3 or can transduce a TCR complex containing CD3. In some embodiments, the cell contains or expresses components of the signaling pathways for transmission of signals from a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some embodiments, the cell or cell line is H9 human T lymphocyte (ATCC, HTB-176) or Jurkat human T cell leukemia cell line (ATCC, TIB-152).

In some embodiments, the cell is a cell line, such as a cell line available from private and commercial sources, such as American Type Culture Collection (ATCC); National Institute of General Medical Sciences (NIGMS); ASHI Repository; the European Collection of Cell Cultures (ECACC); or the International Histocompatibility Working (IHW) Group Cell and DNA bank. In some cases, cell lines are commercially available. In some embodiments, the cells are cell lines or derived from cell lines, e.g., T cell lines. In some embodiments, the cell line is a T lymphocyte or T lymphoblast cell line. For example, the cell or cell line is Jurkat, Clone E6-1 (ATCC, PTS-TIB-152™, TIB-152™); 31E9 (ATCC, HB-11052™); CCRF-CEM (ATCC, CCL-119™, CRM-CCL-119D™, CRM-CCL-119™, PTS-CCL-119™); CCRF-HSB-2 (ATCC, CCL-120.1™); CEM/C1 (ATCC, CRL-2265™); CEM/C2 (ATCC, CRL-2264™); CEM-CM3 (ATCC, TIB-195™); FeT-1C (ATCC, CRL-11968™); FeT-J (ATCC, CRL-11967™); J.CaM1.6 (ATCC, CRL-2063™); J.RT3-T3.5 (ATCC, TIB-153™); J45.01 (ATCC, CRL-1990™); Loucy (ATCC, CRL-2629™); MOLT-3 (ATCC, CRL-1552™); MYA-1 (ATCC, CRL-2417™); SUP-T1 (ATCC, CRL-1942™); TALL-104 (ATCC, CRL-11386™); I 9.2; I 2.1; D1.1; J.gammal subline or J-Lat. In some embodiments, the cell or cell line is Jurkat, Clone E6-1 (ATCC, PTS-TIB-152™, TIB-152™).

a. Engineering Reporter Cells

In some embodiments, the T cells include one or more nucleic acid molecules introduced via genetic engineering, and thereby express recombinant or genetically engineered products of such nucleic acid molecules. In some embodiments, the nucleic acid molecules are heterologous, i.e., normally not present in a cell or sample obtained from the cell, such as one obtained from another organism or cell, which, for example, is not ordinarily found in the cell being engineered and/or an organism from which such cell is derived. In some embodiments, the nucleic acid molecules are not naturally occurring, such as a nucleic acid not found in nature, including one comprising chimeric combinations of nucleic acid molecules encoding various domains from multiple different cell types. In some embodiments, the T cells into which one of a plurality of recombinant receptors are introduced, transfected and/or transduced are T hybridoma cells.

Also provided are plurality of T cells or composition of T cells. In some embodiments, the provided plurality of T cells or composition of T cells comprise any of the T cells described herein, such as reporter T cells. In some embodiments, the provided plurality of T cells or composition of T cells (e.g., reporter T cells) that have been engineered to stably express a fluorescent protein, e.g., a eGFP.

Various methods for the introduction of genetically engineered components, are well known and may be used with the provided methods and compositions. Exemplary methods include those for transfer of nucleic acids and stable expression of corresponding protein, including via viral, e.g., retroviral or lentiviral, transduction, transposons, and electroporation.

In some embodiments, the methods provided herein are used in association with engineering one or more compositions of reporter T cells. In certain embodiments, the engineering is or includes the introduction of a polynucleotide, e.g., a recombinant polynucleotide encoding a recombinant protein. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as Piggy Bac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of reporter T cells.

b. Reporter Molecules

In some embodiments, the cell lines, e.g. T cell lines, contain a reporter molecule or molecules whose expression is responsive to a signal through the intracellular signaling region of the T cell receptor, i.e. hereinafter also called “reporter cells,” such as “reporter T cells”. In some embodiments, the provided cells, such as reporter T cells, contain a reporter molecule or molecules whose expression is responsive to a signal through the intracellular signaling region of the T cell receptor or recombinant receptor. In some embodiments, the expression of the reporter molecule or molecules is responsive to signals through a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some embodiments, expression of the reporter molecule or molecules is responsive to signals through an intracellular signaling domain of a CD3 chain, optionally a CD3-zeta (CD32) chain, or a signaling portion thereof and/or a costimulatory signaling region, such as an intracellular signaling domain of a T cell costimulatory molecule or a signaling portion thereof.

In some embodiments, the provided T cells, e.g., reporter T cells, and/or any of the T cells used to assess vector potency, contain nucleic acid sequences encoding one or more reporter molecules capable of producing a detectable signal or catalyzing measurable activity upon signaling through the intracellular signaling region of the recombinant receptor.

In some embodiments, the detectable signal or measurable activity comprises an indicator that is altered compared to the indicator produced by the reporter molecule(s) in the reporter cell in the absence of vector transduction in the cell, and/or in the presence or absence of an agent that binds to the binding domain of the receptor and/or an agent that induces or is capable of inducing a signal through the intracellular signaling region of the receptor. In some embodiments, the detectable indicator is induced or expressed, increased, decreased, repressed, changed in color or changed in location in the cell compared to the signal produced by the reporter(s) in the absence of vector transduction in the cell, and/or in the presence or absence of an agent that binds to the binding domain of the receptor and/or an agent that induces or is capable of inducing a signal through the intracellular signaling region of the receptor. In some embodiments, the expression of the reporter molecule(s) is responsive to the quality and/or strength of the signal through the intracellular signaling region and/or binding and/or recognition of the recombinant receptor to a target antigen or epitope. Thus, in some embodiments, the reporter(s) capable of producing an indictor upon signal through the intracellular signaling region of the recombinant receptor, can be used in low-, medium- or high-throughput screening methods to determine the potency, e.g., transduction efficiency, of the vector introduced into the T cells or plurality of T cells.

In some embodiments, the reporter(s) are capable of being detected, such as expressed or induced into catalytic activity, in the cell upon signaling through the intracellular signaling region and/or binding and/or recognition of the recombinant receptor to a target antigen or epitope and/or upon cell signaling transduced through an intracellular signaling region containing CD3 or a portion thereof. In general, a signal, such as a T cell receptor activation signal, is induced or initiated upon binding of an agent, e.g., specific antigen or epitope, which leads to the cross-linking and activation of the signaling complex that contains CD3. The signal, in some cases, then can initiate further downstream signaling and expression of various intracellular compounds associated with antigen or epitope binding and/or activation signaling, e.g., T cell activation signaling. In some embodiments, T cell activation through the CD3 complex can lead to induction of signal transduction pathways in the T cell resulting in production of cellular signaling and expression of products (e.g., interleukin-2) by that T cell.

In some embodiments, a “reporter molecule” or “reporter” is any molecule that is or can produce a detectable signal that is altered compared to the signal from or produced by the reporter in the presence or absence of an agent that binds to the binding domain of the receptor and/or an agent that induces or is capable of inducing a signal through the intracellular signaling region of the recombinant receptor, and/or in the absence of T cell activation, e.g., T cell activation through the intracellular signaling region of the receptor. In some embodiments, the detectable signal is induced or expressed, increased, decreased, repressed, changed in color or changed in location in the cell compared to the signal produced by the reporter in the absence of T cell activation and/or in the absence of the recombinant receptor in the cell. In some embodiments, the reporter is or can produce a detectable signal in the cell that can include light emission (e.g. fluorescence), FRET, concentration of a biochemical second messenger, i.e. molecule (e.g. calcium), protein or gene expression in the cell or protein secretion from the cell (e.g. IL-2). In some embodiments, the reporter is an enzyme or can catalyze a reaction within the cell that produces measurable product or products. Various reporter systems of T cell function, including T cell activation, are known (see e.g. Hoekstra et al. (2015) Trends in Immunol, 36:392-400).

In some embodiments, the reporter is a detectable moiety, such as a light-emitting protein or bioluminescent protein, that can be detectable and can be monitored visually, or by using a spectrophotometer, luminometer, fluorometer or other related methods. In some embodiments, the reporter is a detectable moiety, such as an enzyme that produces bioluminescence, e.g., enzymes that can convert a substrate that emits light, e.g., luciferase or variants thereof. Non-limiting examples of light emitting proteins or enzymes that produce bioluminescence include, for example, luciferase, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS), and fluorescent protein and variants thereof, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. Luciferases and variants thereof can include luciferases from the firefly (Photinus pyralis), sea pansy (Renilla reniformis), Photobacterium species (Vibrio fischeri, Vibrio haweyi and Vibrio harveyi), dinoflagellates, marine copepod (Metridia longa), deep sea shrimp (Oplophorus) and Jack-O-Lantern mushroom (Omphalotus olearius), and variants thereof, including codon-optimized and/or enhanced variants. In some embodiments, the reporter molecule is a firefly luciferase, optionally firefly luciferase 2 (amino acid sequence set forth in SEQ ID NO: 9, encoded by nucleic acid sequence set forth in SEQ ID NO:8). In some embodiments, the reporter molecule is a green fluorescent protein (GFP), optionally enhanced GFP (amino acid sequence set forth in SEQ ID NO: 11, encoded by nucleic acid sequence set forth in SEQ ID NO:10).

In some embodiments, the reporter molecule can be a hormone or cytokines or other such well-known genes that can be induced or expressed in a T cell upon antigen or epitope binding and/or activity of a receptor, e.g., signaling or activation. The expression of these reporter genes can also be monitored by measuring levels of mRNA transcribed from these genes.

In some embodiments, a reporter, such as a detectable moiety, can be directly associated with a particular recombinant receptor, e.g., CAR, or downstream signal induced by activation of the recombinant receptor, e.g., CAR, following antigen or epitope binding, thereby providing a direct read-out of activity of the reporter, e.g., signaling or cell activation. In some embodiments, the detectable signal in the cell induced upon antigen or epitope binding and/or signal or activity through the intracellular signaling region of the recombinant receptor, is a change in location of the detectable moiety in the cell compared to its location in the cell in the absence of binding of the antigen receptor to a recognized antigen or epitope, and/or signal or activity through the intracellular signaling region of the recombinant receptor. In some aspects, a particular recombinant receptor, e.g., CAR, can be engineered with, such as operably fused to, a detectable moiety whose activity is turned on and/or can be otherwise visualized upon engagement or binding to an antigen, such as an epitope. In some cases, engagement of the recombinant receptor, e.g., CAR, can result in internalization of the receptor, which can be monitored. In some embodiments, a transcription factor or other signaling molecule whose expression is induced in response to signal or activity through the intracellular signaling region of the recombinant receptor can be engineered with, such as operably fused to, a detectable moiety whose activity is turned on and/or can be otherwise visualized upon engagement of binding to an antigen or epitope. In some cases, signal or activity through the intracellular signaling region of the recombinant receptor, such as T cell activation and/or signaling, can result in translocation of the signal-specific transcription factor from the cytosol to the nucleus, which can be monitored. In some embodiments, the detectable moiety can be any as described, such as a fluorescent, enzymatic or luminescent protein.

In some embodiments, fluorescence resonance energy transfer (FRET) based systems can be used that monitor changes in the interactions between two molecules in the cell. FRET systems that can monitor TCR engagement and/or T cell activation are known (see e.g., Zal and Gascoigne (2004) Curr. Opin. Immunol., 16:674-83; Yudushkin and Vale (2010) PNAS, 107:22128-22133; Ibraheem et al. (2010) Curr. Opin. Chem. Biol., 14:30-36).

In some embodiments of the methods and cells provided herein, the reporter molecule(s) are associated with, under operable control of and/or regulated by a T cell activation factor. In some embodiments, the reporter molecule is encoded by a nucleic acid sequence under the operable control of a T cell activation factor, e.g., a regulatory element that is responsive to the quality and/or strength of the signal through the intracellular signaling region and/or binding and/or recognition of the recombinant receptor to a target antigen or epitope. In some embodiments, a “T cell activation factor” is a molecule or factor or portion thereof that is responsive to antigen or epitope binding by a receptor, e.g. T cell receptor (TCR) present or expressed on a T cell or to a signal transduced through a components of the TCR complex of a T cell, or a recombinant receptor comprising intracellular signaling regions that comprise a component of the TCR complex or a portion thereof. In some embodiments, the T cell activation factor can be a canonical factor or a portion thereof that is part of the normal downstream signaling pathway of T cells. In some embodiments, the read-out of T cell activation is a reporter encoded by a construct containing a T cell activation factor operably connected to the reporter molecule capable of detectable expression. In some embodiments, antigen or epitope binding and/or signal or activity through the intracellular signaling region of the recombinant receptor, e.g., CAR induces signaling that induces the T cell activation factor to express the reporter. Detectable expression of the reporter molecule can then be monitored as an indicator of T cell activation.

In some embodiments, the T cell activation factor is or contains one or more regulatory elements, such as one or more transcriptional control elements, of a target gene whose expression depends on or is associated with activation of components of the TCR complex, whereby the regulatory domain or element is recognized by a transcription factor to drive expression of such gene. In some cases, the T cell activation factor, such as a regulatory domain or element, can be or contain all or a portion of an endogenous regulatory region of a particular gene locus, e.g. the T cell activation factor is derived from a target gene locus. In some embodiments, the T cell activation factor is or contains a promoter, enhancer or other response element or portion thereof, recognized by a transcription factor to drive expression of a gene whose activity is normally turned on by T cell activation. In some embodiments, the T cell activation factor can be a regulatory domain or region (e.g. promoter, enhancer or other response element) of a transcription factor whose activity is turned on by T cell activation. In some embodiments, the T cell activation factor is responsive to one or more of the quality and/or strength of the signal through the intracellular signaling region and/or binding and/or recognition of the recombinant receptor to a target antigen or epitope. In some embodiments, the regulatory element is responsive to one or more of the state of the recombinant receptor binding to an antigen or epitope, T cell activation, signal strength of the recombinant receptor and/or quality of the signaling through the intracellular signaling region of the recombinant receptor, e.g., CAR. In some embodiments, the T cell activation factor is or comprises a transcriptional regulatory element of a gene whose expression is induced and/or is upregulated upon binding of the recombinant receptor binding to an antigen or epitope, T cell activation, signal strength of the recombinant receptor and/or quality of the signaling through the intracellular signaling region of the recombinant receptor, e.g., CAR.

Typically, a T cell activation factor is operably associated with a detectable readout of T cell activation, such as a reporter that is expressed from the cell and can be detected. Thus, for example, the expression of the reporter, instead of or in addition to the endogenous gene, can be induced upon T cell activation. The T cell activation factor, alone or together with a detectable readout, can be endogenous, exogenous or heterologous to the cell.

In some embodiments, the T cell activation factor can be a regulatory element, such as a transcriptional regulatory element, such as promoter, enhancer or response element or elements, that contain a binding site for a T cell transcription factor, and that thereby is associated with the downstream activity of a T cell transcription factor. In some embodiments, the transcription factor is nuclear factor of activated T cells (NFAT), C/EBP, AP1, STAT1, STAT2, Nur77 or NFκB. In some embodiments, the T cell activation factor contains a response element or elements recognized by a nuclear factor of activated T cells (NFAT), C/EBP, AP1, STAT1, STAT2, Nur77 and NFκB. In some embodiments, the T cell activation factor can contain a regulatory element or elements recognized by or responsive to one or two, and in some cases three or more, unique transcription factors.

In some cases, the T cell activation factor contains a binding site, such as a response element, recognized by only a single transcription factor that is selectively activated by signaling through components of the TCR complex induced through receptor engagement following antigen or epitope binding to the receptor, e.g., recombinant receptor, e.g., CAR. In some embodiments, the T cell activation factor comprises a response element or elements recognized by a transcription factor that is activated upon stimulation of T cells through an endogenous TCR complex. For example, generally regulatory regions of genes contain multiple regulatory elements that can be responsive to more than one signaling pathway in a cell. In contrast, an artificial regulatory region or artificial promoter that contains a regulatory element or elements recognized by a transcription factor selectively activated by signaling only through the components of the TCR complex can increase the specificity of the reporter system so that it is responsive only to T cell activation. In some embodiments, the T cell activation factor contains a regulatory element or elements recognized by NFAT. In some embodiments, the T cell activation factor contains a regulatory element or elements recognized by NFκB.

In some embodiments, the reporter molecule is encoded by a nucleic acid sequence under the operable control of a T cell activation factor, such as a regulatory element that is responsive to the quality and/or strength of the signal through an antigen receptor such as a TCR complex. In some aspects the T cell activation factor is responsive to the quality and/or strength of signal through the intracellular signaling region of, and/or in response to the binding to and/or recognition of a recombinant receptor (such as the receptor being screened or assessed, such as the recombinant receptor expressed by the cell) a target antigen or epitope. In some aspects, the T cell activation factor is or contains a transcriptional regulatory element or elements associated with the expression of the orphan nuclear hormone receptor Nur77 (also called Nr4a1, nerve growth factor IB (NGFIB), GFRP1; Gfrp; HMR; Hbr-1; Hbr1; Hmr; N10; NAK-1; NGFI-B; NGFIB; NP10; Ngfi-b; Orphan nuclear receptor HMR; ST-59; TIS1; TR3; TR3 orphan receptor; early response protein NAK1; growth factor-inducible nuclear protein N10; hormone receptor; immediate early gene transcription factor NGFI-B; nerve growth factor IB nuclear receptor variant 1; nerve growth factor induced protein I-B; nerve growth factor-induced protein I-B; neural orphan nuclear receptor NUR77; nhr-6; nr4a1; nuclear hormone receptor NUR/77; nuclear protein N10; nuclear receptor subfamily 4 group A member 1; orphan nuclear receptor NGFI-B; orphan nuclear receptor NR4A1; orphan nuclear receptor TR3; steroid receptor TR3; testicular receptor 3; zgc: 92434; exemplary human Nur77 DNA sequence set forth in SEQ ID NO: 1, encoding the polypeptide set forth in SEQ ID NO:2).

Nur77 generally is encoded by an immediate-early response gene induced in response to signaling through, or activation of signal from, the endogenous T cell receptor (TCR) complex, engagement of the endogenous TCR and/or via molecules containing immunoreceptor tyrosine-based activation motif (ITAM) that are involved in the signal from the TCR complex, e.g., CD3-zeta signaling regions. Nur77 gene product itself generally can bind to regulatory elements associated with the promoters of several genes to induce downstream expression of genes. The level or extent of expression of Nur77 can serve as an indicator for strength of T cell signals, e.g., TCR signals (Moran et al. (2011) JEM, 208:1279-1289). Thus, in some embodiments, expression of a reporter molecule operably connected to a transcriptional regulatory element or elements of the Nur77 gene locus, or portion thereof, can provide an indicator of the strength of T cells signaling. Further, Nur77 expression is generally not affected or influenced by other signaling pathways such as cytokine signaling or toll-like receptor (TLR) signaling (see, e.g., Ashouri et al., (2017) J. Immunol. 198:657-668), which may act in a cell extrinsic manner and may not depend on signaling through the recombinant receptor. In some embodiments, the T cell activation factor is a Nur77 promoter or enhancer or a portion thereof, or is a molecule or gene that contains a Nur77 response element or elements.

In some of any of the embodiments, the reporter T cells contain a nucleic acid sequence encoding a reporter molecule operably linked to a transcriptional regulatory element of a Nur77, or a variant thereof. In some of any of such embodiments, the variant of the transcriptional regulatory element is a variant nucleic acid sequence that exhibits at least 85%, 86%, 87%, 88%, 89%, 90%, 91%, 92%, 93%, 94%, 95%, 96%, 97%, 98%, 99% or more sequence identity to a transcriptional regulatory element within an endogenous Nur77 locus in the T cell. In some of any of such embodiments, the variant of the transcriptional regulatory element is a functional variant, having a nucleic acid sequence that exhibits at least 85%, 86%, 87%, 88%, 89%, 90%, 91%, 92%, 93%, 94%, 95%, 96%, 97%, 98%, 99% or more sequence identity to a transcriptional regulatory element within an endogenous Nur77 locus in the T cell and is responsive to signaling through, or signal from, the endogenous T cell receptor (TCR) complex, engagement of the endogenous TCR and/or via molecules containing immunoreceptor tyrosine-based activation motif (ITAM) that are involved in the signal from the TCR complex, e.g., CD3-zeta signaling regions; and/or is responsive to a signal through the intracellular signaling region of the recombinant receptor, wherein the incubating is carried out in the presence or absence of an agent that binds to the binding domain of the recombinant receptor and/or an agent that induces or is capable of inducing a signal through the intracellular signaling region of the recombinant receptor.

In some embodiments, a construct or vector is generated that contains nucleic acid sequences encoding a reporter molecule under the operable control of a T cell activation factor, e.g., Nur77 promoter, capable of being activated or induced upon antigen or epitope binding and/or signal or activity through the intracellular signaling region of the receptor, e.g., recombinant receptor e.g., CAR, to a recognized an antigen or an epitope thereof. In some embodiments, “a reporter construct” comprises a nucleic acid that encodes reporter molecule(s) operatively linked to sequences for a T cell activation factor or factors that is/are capable of inducing its expression.

Reporter constructs are known or can be generated by recombinant DNA techniques. In some embodiments, the nucleic acid sequences encoding a reporter molecule or molecules is cloned into an expression plasmid, such as a mammalian expression vector, for example pcDNA or other mammalian expression vector. In some embodiments, the nucleic acid sequences encoding a reporter molecule or molecules is cloned into a retroviral vector, e.g. lentiviral vector.

In some embodiments, the nucleic acid sequences encoding a reporter molecule or molecules is integrated into a genomic location in the cell, e.g., an endogenous genomic location. In some embodiments, the nucleic acid sequences encoding a reporter molecule can be integrated into a genomic location for its expression to be associated with, under operable control of and/or regulated by the regulatory elements present in the endogenous genomic location of a particular gene whose expression can be responsive to the quality and/or strength of the signal through the intracellular signaling region and/or binding and/or recognition of the receptor to a target antigen or epitope, and/or T cell signaling or T cell activation. In some embodiments, the nucleic acid sequences encoding a reporter molecule or molecules can be integrated into an endogenous genomic location, placed under the operative control of a transcriptional regulatory element of a gene whose expression is induced and/or is upregulated upon signal through the intracellular signaling region of the recombinant receptor and/or binding and/or recognition of the recombinant receptor to a target antigen or epitope. In some embodiments, the nucleic acid sequences encoding a reporter molecule or molecules can be integrated into an endogenous genomic location for co-expression with the endogenous gene encoded at the location, which is under operable control of a T cell activation factor, e.g., a promoter, an enhancer or a response element or a portion thereof, capable of being activated or induced upon antigen or epitope binding and/or signal or activity through the intracellular signaling region of the recombinant receptor, e.g., CAR, to a recognized an antigen or an epitope thereof and/or T cell signaling or T cell activation. In some embodiments, the endogenous gene is Nur77. In some embodiments, the T cell activation factor is the Nur77 promoter, enhancer or response element or a portion thereof. In some embodiments, the nucleic acid sequences encoding a reporter molecule is targeted for integration in-frame with the coding sequence, coding region and/or open reading frame (ORF) of the endogenous gene, e.g., the endogenous Nur77 gene, separated by sequences encoding a self-cleavage element, e.g., T2A.

In some embodiments, the T cells or plurality of T cells provided herein or the T cells or plurality of T cells used in the methods provided herein can contain more than one reporters. In some embodiments, the T cells or plurality of T cells can contain two different reporters.

c. Exemplary Reporter T Cells

In some embodiments, the provided reporter T cells or the reporter T cells used in the methods provided herein, contain nucleic acid sequences encoding a reporter molecule is present within the genome of the cell or is targeted for integration into an endogenous genomic location, such that the expression of the reporter can be associated with, under operable control of and/or regulated by the regulatory elements present in the endogenous genomic location of a particular gene whose expression can be responsive to the quality and/or strength of the signal through the intracellular signaling region and/or binding and/or recognition of the recombinant receptor to a target antigen or epitope, and/or T cell signaling or T cell activation. In some embodiments, the reporter T cell is generated by inducing a genetic disruption at one or more target site(s) at or near the endogenous locus of interest; and introducing a template polynucleotide for homology directed repair (HDR). In some embodiments, the reporter T cells contain a targeted knock-in of nucleic acid sequences encoding a reporter molecule at an endogenous locus that is linked to a T cell activation factor, such as a regulatory element that is responsive to the quality and/or strength of the signal through an endogenous T cell receptor (TCR) and/or binding and/or recognition of the TCR to a target antigen or epitope.

In some embodiments, the reporter T cell is generated by inducing a targeted genetic disruption, e.g., generation of a DNA break, using gene editing methods, followed by HDR for a targeted knock-in of the nucleic acid sequences encoding a reporter molecule at the endogenous locus linked to a T cell activation factor, such as the Nur77 promoter, enhancer or response element or a portion thereof. In some embodiments, the nucleic acid sequences encoding a reporter molecule is present within the genome of the cell or is targeted for integration in-frame with the coding sequence, coding region and/or open reading frame (ORF) of the endogenous gene, e.g., the endogenous Nur77 gene. Thus, in some exemplary embodiments, the reporter T cell is generated by inducing a genetic disruption at one or more target site(s) at or near the endogenous locus encoding Nur77; and introducing a template polynucleotide for HDR.

In some embodiments, the genetic disruption is induced by a DNA binding protein or DNA-binding nucleic acid that specifically binds to or hybridizes to the target site, optionally a fusion protein comprising a DNA-targeting protein and a nuclease or an RNA-guided nuclease. In some embodiments, the fusion protein comprising a DNA-targeting protein and a nuclease or the RNA-guided nuclease is or comprises a zinc finger nuclease (ZFN), a TAL-effector nuclease (TALEN), or a CRISPR-Cas9 combination that specifically binds to, recognizes, or hybridizes to the target site. In some embodiments, the RNA-guided nuclease comprises a guide RNA (gRNA) having a targeting domain that is complementary to the target site.

In some embodiments, the introduction of a genetic disruption or cleavage involve the use of one or more agent(s) capable of introducing a genetic disruption, a cleavage, a double strand break (DSB) and/or a nick at a target site in the genomic DNA, thereby activating and/or recruiting various cellular DNA repair mechanisms, which can utilize the template polynucleotide, containing homology arm sequences, a DNA repair template, to effectively copy and integrate the nucleic acid sequences encoding the reporter molecule, at or near the site of the targeted genetic disruption by HDR, based on homology between the endogenous gene sequence surrounding the target site and the 5′ and/or 3′ homology arms included in the template polynucleotide.

In some embodiments, the one or more agent(s) capable of introducing a genetic disruption or cleavage comprises a DNA binding protein or DNA-binding nucleic acid that specifically binds to or hybridizes to a target site in the genome, e.g., at or near the Nur77 gene. In some aspects, the targeted cleavage, e.g., DNA break, at or near the endogenous gene encoding Nur77 is achieved using a protein or a nucleic acid is coupled to or complexed with a gene editing nuclease, such as in a chimeric or fusion protein. In some embodiments, the one or more agent(s) capable of introducing a genetic disruption or cleavage comprises a fusion protein comprising a DNA-targeting protein and a nuclease or an RNA-guided nuclease.

In some embodiments, introducing a genetic disruption or cleavage is carried out by gene editing methods, such as using a zinc finger nuclease (ZFN), TALEN or a CRISPR/Cas system with an engineered guide RNA that cleaves the target site(s), e.g., target site(s) at or near the Nur77 gene.

In some embodiments, the agent capable of introducing a targeted cleavage comprises various components, such as a fusion protein comprising a DNA-targeting protein and a nuclease or an RNA-guided nuclease. In some embodiments, the targeted cleavage is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted cleavage is carried out using RNA-guided nucleases such as a clustered regularly interspaced short palindromic nucleic acid (CRISPR)-associated nuclease (Cas) system (including Cas and/or Cfp1). In some embodiments, the targeted cleavage is carried using agents capable of introducing a genetic disruption or cleavage, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically engineered and/or designed to be targeted to the at least one target site(s), sequence of a gene or a portion thereof.

In some embodiments, the one or more agent(s) specifically targets the at least one target site(s), e.g., at or near the Nur77 gene. In some embodiments, the agent comprises a ZFN, TALEN or a CRISPR/Cas9 combination that specifically binds to, recognizes, or hybridizes to the target site(s). In some embodiments, the CRISPR/Cas9 system includes an engineered crRNA/tracr RNA (“single guide RNA”) to guide specific cleavage. In some embodiments, the agent comprises nucleases based on the Argonaute system (e.g., from T. thermophilus, known as ‘TtAgo’, (Swarts et at (2014) Nature 507 (7491): 258-261).

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073. Exemplary ZFNs, TALEs, and TALENs are described in, e.g., Lloyd et al., Frontiers in Immunology, 4 (221): 1-7 (2013).

A zinc finger protein (ZFP) or zinc finger domain thereof is a protein or domain within a larger protein that binds DNA in a sequence-specific manner through one or more zinc fingers, regions of amino acid sequence within the binding domain whose structure is stabilized through coordination of a zinc ion. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of targets. See, e.g., Gaj et al., Trends in Biotechnology, 2013, 31 (7), 397-405. In some cases, commercially available zinc fingers are used or are custom designed.

In some embodiments, the Nur77 gene can be targeted for cleavage using clustered regularly interspaced short palindromic repeats (CRISPR) and CRISPR-associated (Cas) proteins. See Sander and Joung, Nature Biotechnology, 32 (4): 347-355. In some embodiments, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9), with nuclease functionality. In some embodiments, the CRISPR/Cas nuclease system comprises at least one of: a guide RNA (gRNA) having a targeting domain that is complementary with a target site of a Nur77 gene; or at least one nucleic acid encoding the gRNA.

In general, a guide sequence, e.g., guide RNA, is any polynucleotide sequences comprising at least a sequence portion, e.g., targeting domain, that has sufficient complementarity with a target site sequence, such as a target site in the Nur77 gene in humans, to hybridize with the target sequence at the target site and direct sequence-specific binding of the CRISPR complex to the target sequence. In some embodiments, in the context of formation of a CRISPR complex, “target site” (also known as “target position,” “target DNA sequence” or “target location”) generally refers to a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some aspects, a CRISPR enzyme (e.g. Cas9 nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a guide RNA (gRNA) specific to the target site (e.g. the Nur77 gene) is used to guide RNA-guided nucleases, e.g., Cas, to introduce a DNA break at the target site or target position. Methods for designing gRNAs and exemplary targeting domains can include those described in, e.g., in International PCT Publication No. WO2015/161276. Targeting domains can be incorporated into the gRNA that is used to target Cas9 nucleases to the target site or target position. Methods for selection and validation of target sequences as well as off-target analyses are described, e.g., in Mali et al., 2013 Science 339 (6121): 823-826; Hsu et al. Nat Biotechnol, 31 (9): 827-32; Fu et al., 2014 Nat Biotechnol; Heigwer et al., 2014 Nat Methods 11 (2): 122-3; Bae et al., 2014 Bioinformatics; Xiao A et al., 2014 Bioinformatics. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some exemplary embodiments, the target site is at or near the final exon of the endogenous locus encoding Nur77. In some exemplary embodiments, the target site is at or near the final exon of the endogenous locus encoding Nur77 but prior to the stop codon of the endogenous locus encoding Nur77. In some embodiments, the one or more target site(s) comprise the nucleic acid sequence TCATTGACAAGATCTTCATG (SEQ ID NO:14) and/or GCCTGGGAACACGTGTGCA (SEQ ID NO:15). In some embodiments, the gRNA comprises a targeting domain sequence selected from CAUGAAGAUCUUGUCAAUGA (SEQ ID NO:3) or UGCACACGUGUUCCCAGGC (SEQ ID NO:4).

In some embodiments, induction of genetic disruption or cleavage is carried out by delivering or introducing one or more agent(s) capable of introducing a genetic disruption or cleavage, e.g., Cas9 and/or gRNA components, to a cell, using any of a number of known delivery method or vehicle for introduction or transfer to cells, for example, using lentiviral delivery vectors, or any of the known methods or vehicles for delivering Cas9 molecules and gRNAs. Exemplary methods are described in, e.g., Wang et al. (2012) J. Immunother. 35 (9): 689-701; Cooper et al. (2003) Blood. 101:1637-1644; Verhoeyen et al. (2009) Methods Mol Biol. 506:97-114; and Cavalieri et al. (2003) Blood. 102 (2): 497-505. In some embodiments, nucleic acid sequences encoding one or more components of one or more agent(s) capable of introducing a genetic disruption or cleavage, e.g., DNA break, is introduced into the cells, e.g., by any methods for introducing nucleic acids into a cell described herein or known. In some embodiments, a vector encoding components of one or more agent(s) capable of introducing a genetic disruption or cleavage such as a CRISPR guide RNA and/or a Cas9 enzyme can be delivered into the cell.

In some embodiments, the one or more agent(s) capable of introducing a genetic disruption or cleavage, e.g., a Cas9/gRNA system, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, calcium phosphate transfection, cell compression or squeezing. In some embodiments, the RNP can cross the plasma membrane of a cell without the need for additional delivery agents (e.g., small molecule agents, lipids, etc.).

In some embodiments, a template polynucleotide comprising nucleic acid sequences encoding the reporter molecule is introduced into the cell. In some embodiments, a template polynucleotide is introduced into the engineered cell, prior to, simultaneously with, or subsequent to introduction of agent(s) capable of inducing a targeted genetic disruption. In the presence of a targeted genetic disruption, e.g., DNA break, the template polynucleotide can be used as a DNA repair template, to effectively copy and integrate the transgene, e.g., nucleic acid sequences encoding the reporter molecule, at or near the site of the targeted genetic disruption by HDR, based on homology between the endogenous gene sequence surrounding the target site and the 5′ and/or 3′ homology arms included in the template polynucleotide. In some embodiments, the gene editing and HDR steps are performed simultaneously and/or in one experimental reaction. In some embodiments, the gene editing and HDR steps are performed consecutively or sequentially, in one or consecutive experimental reaction(s). In some embodiments, the gene editing and HDR steps are performed in separate experimental reactions, simultaneously or at different times.

In some embodiments, HDR can be utilized for targeted integration of one or more transgene at one or more target site in the genome, e.g., the Nur77 gene. In some embodiments, the nuclease-induced HDR can be used to alter a target sequence, integrate a transgene, e.g., nucleic acid sequences encoding a reporter molecule, at a particular target location.

Alteration of nucleic acid sequences at the target site can occur by HDR with an exogenously provided template polynucleotide (also referred to as donor polynucleotide or template sequence). For example, the template polynucleotide provides for alteration of the target sequence, such as insertion of the transgene contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Template polynucleotide-effected alteration of a target sequence depends on cleavage by a nuclease, e.g., a targeted nuclease such as CRISPR/Cas9. Cleavage or genetic disruption by the nuclease can comprise a double strand break or two single strand breaks.

In some embodiments, “recombination” refers to a process of exchange of genetic information between two polynucleotides. In some embodiments, “homologous recombination (HR)” refers to the specialized form of such exchange that takes place, for example, during repair of double-strand breaks in cells via homology-directed repair mechanisms. This process requires nucleotide sequence homology, uses a template polynucleotide to template repair of a target DNA (i.e., the one that experienced the double-strand break, e.g., target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide.

In some embodiments, a template polynucleotide, e.g., polynucleotide containing transgene, is integrated into the genome of a cell via homology-independent mechanisms. The methods comprise creating a double-stranded break (DSB) in the genome of a cell and cleaving the template polynucleotide molecule using a nuclease, such that the template polynucleotide is integrated at the site of the DSB. In some embodiments, the template polynucleotide is integrated via non-homology dependent methods (e.g., NHEJ). Upon in vivo cleavage the template polynucleotides can be integrated in a targeted manner into the genome of a cell at the location of a DSB. The template polynucleotide can include one or more of the same target sites for one or more of the nucleases used to create the DSB. Thus, the template polynucleotide may be cleaved by one or more of the same nucleases used to cleave the endogenous gene into which integration is desired. In some embodiments, the template polynucleotide includes different nuclease target sites from the nucleases used to induce the DSB. As described above, the genetic disruption of the target site or target position can be created by any mechanisms, such as ZFNs, TALENs, CRISPR/Cas9 system, or TtAgo nucleases.

In canonical HDR, a double-stranded template polynucleotide is introduced, comprising a homologous sequence to the target site that will either be directly incorporated into the target site or used as a template to insert the transgene near the target site. After resection at the genetic disruption or cleavage, repair can progress by different pathways, e.g., by the double Holliday junction model (or double strand break repair, DSBR, pathway) or the synthesis-dependent strand annealing (SDSA) pathway. In some embodiments, other DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

Targeted integration results in the transgene being integrated into a specific gene or locus in the genome. The transgene may be integrated anywhere at or near one of the at least one target site(s) or site in the genome. In some embodiments, the transgene is present within the genome of the cell or present within the genome of the cell or integrated at or near one of the at least one target site(s), for example, within 300, 250, 200, 150, 100, 50, 10, 5, 4, 3, 2, 1 or fewer base pairs upstream or downstream of the site of cleavage, such as within 100, 50, 10, 5, 4, 3, 2, 1 base pairs of either side of the target site, such as within 50, 10, 5, 4, 3, 2, 1 base pairs of either side of the target site.

The genetic disruption or cleavage at the target site should be sufficiently close to the site for targeted integration such that an alteration is produced in the desired region, e.g., insertion of transgene occurs. In some embodiments, the distance is not more than 10, 25, 50, 100, 200, 300, 350, 400 or 500 nucleotides. In some embodiments, it is believed that the genetic disruption or cleavage should be sufficiently close to the site for targeted integration such that the genetic disruption or cleavage is within the region that is subject to exonuclease-mediated removal during end resection. In some embodiments, the targeting domain is configured such that a cleavage event, is positioned within 1, 2, 3, 4, 5, 10, 15, 20, 25, 30, 35, 40, 45, 50, 60, 70, 80, 90, 100, 150, 200, 300, 350, 400 or 500 nucleotides of the region desired to be altered, e.g., site for targeted insertion, such as between about 0 and about 200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the site for targeted integration. The genetic disruption or cleavage can be positioned upstream or downstream of the region desired to be altered, e.g., site for targeted insertion. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of site for targeted integration.

A template polynucleotide having homology with sequences at or near one or more target site(s) in the endogenous DNA can be used to alter the structure of a target DNA, e.g., targeted insertion of the transgene, e.g., nucleic acid sequences encoding a reporter molecule. In some embodiments, the template polypeptide contains homology sequences (e.g., homology arms) flanking the transgene, e.g., nucleic acid sequences encoding a reporter molecule, such as any reporter molecules described herein, for targeted insertion. In some embodiments, the homology sequences target the transgene at or near the Nur77 locus. In some embodiments, the template polynucleotide includes additional sequences (coding or non-coding sequences) between the homology arms, such as a regulatory sequences, such as promoters and/or enhancers, splice donor and/or acceptor sites, internal ribosome entry site (IRES), sequences encoding ribosome skipping elements (e.g., 2A peptides), markers and/or SA sites, and/or one or more additional transgenes. The sequence of interest in the template polynucleotide may comprise one or more sequences encoding a functional polypeptide (e.g., a cDNA), with or without a promoter.

In some embodiments, nuclease-induced HDR results in an insertion of a transgene (also called “exogenous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

Polynucleotides for insertion can also be referred to as “transgene” or “exogenous sequences” or “donor” polynucleotides or molecules. The template polynucleotide can be DNA, single-stranded and/or double-stranded and can be introduced into a cell in linear or circular form. See also, U.S. Patent Publication Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by methods known. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Publication No. 20130326645.

In some embodiments, the template polynucleotide is double stranded. In some embodiments, the template polynucleotide is single stranded. In some embodiments, the template polynucleotide comprises a single stranded portion and a double stranded portion.

In some embodiments, the template polynucleotide contains the transgene, e.g., reporter molecule-encoding nucleic acid sequences, flanked by homology sequences (also called “homology arms”) on the 5′ and 3′ ends, to allow the DNA repair machinery, e.g., homologous recombination machinery, to use the template polynucleotide as a template for repair, effectively inserting the transgene into the target site of integration in the genome. The homology arm should extend at least as far as the region in which end resection may occur, e.g., in order to allow the resected single stranded overhang to find a complementary region within the template polynucleotide. The overall length could be limited by parameters such as plasmid size or viral packaging limits. In some embodiments, a homology arm does not extend into repeated elements, e.g., ALU repeats or LINE repeats.

Exemplary homology arm lengths include at least or at least about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides.

Target site (also known as “target position,” “target DNA sequence” or “target location”), in some embodiments, refers to a site on a target DNA (e.g., the chromosome) that is modified by the one or more agent(s) capable of inducing a genetic disruption, e.g., a Cas9 molecule. For example, the target site can be a modified Cas9 molecule cleavage of the DNA at the target site and template polynucleotide directed modification, e.g., targeted insertion of the transgene, at the target site. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence (e.g., the sequence to which the gRNA binds).

In some embodiments, the template polynucleotide comprises about 500 to 1000, e.g., 600 to 900 or 700 to 800, base pairs of homology on either side of the target site at the endogenous gene. In some embodiments, the template polynucleotide comprises about 500, 600, 700, 800, 900 or 1000 base pairs homology 5′ of the target site, 3′ of the target site, or both 5′ and 3′ of the target site.

In some embodiments, a template polynucleotide is to a nucleic acid sequence which can be used in conjunction with a nuclease, e.g., Cas9 molecule, and/or a gRNA molecule to alter the structure of a target site. In some embodiments, the target site is modified to have some or all of the sequence of the template polynucleotide, typically at or near cleavage site(s). In some embodiments, the template polynucleotide is single stranded. In some embodiments, the template polynucleotide is double stranded. In some embodiments, the template polynucleotide is DNA, e.g., double stranded DNA In some embodiments, the template polynucleotide is single stranded DNA. In some embodiments, the template polynucleotide is encoded on the same vector backbone, e.g. AAV genome, plasmid DNA, as the Cas9 and gRNA. In some embodiments, the template polynucleotide is excised from a vector backbone in vivo, e.g., it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, e.g., in a vector.

In some embodiments, the template polynucleotide alters the structure of the target site, e.g., insertion of transgene, by participating in a homology directed repair event. In some embodiments, the template polynucleotide alters the sequence of the target site.

In some embodiments, the template polynucleotide includes sequence that corresponds to a site on the target sequence that is cleaved by a Cas9-mediated cleavage event. In some embodiments, the template polynucleotide includes sequence that corresponds to both, a first site on the target sequence that is cleaved in a first Cas9 mediated event, and a second site on the target sequence that is cleaved in a second Cas9 mediated event.

A template polynucleotide typically comprises the following components: [5′ homology arm]-[transgene]-[3′ homology arm]. The homology arms provide for recombination into the chromosome, thus insertion of the transgene into the DNA at or near the cleavage site e.g., target site(s). In some embodiments, the homology arms flank the most distal cleavage sites.

In some embodiments, the template polynucleotide comprises the structure [5′ homology arm]-[nucleic acid sequence encoding the reporter molecule]-[3′ homology arm]. In some embodiments, the 5′ homology arm and/or 3′ homology arm comprises nucleic acid sequences homologous to nucleic acid sequences present at and/or surrounding the one or more target site(s).

In some embodiments, the 5′ homology arm comprises nucleic acid sequences that are homologous to nucleic acid sequences 5′ of the one or more target site(s). In some embodiments, the 3′ homology arm comprises nucleic acid sequences that are homologous to nucleic acid sequences 3′ of the one or more target site(s). In some embodiments, the 5′ homology arm and 3′ homology arm independently is between about 50 and 100, 100 and 250, 250 and 500, 500 and 750, 750 and 1000, 1000 and 2000 base pairs in length.

In some embodiments, the 3′ end of the 5′ homology arm is the position next to the 5′ end of the transgene. In some embodiments, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the transgene. In some embodiments, the 5′ end of the 3′ homology arm is the position next to the 3′ end of the transgene. In some embodiments, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate. In some embodiments, for targeted insertion, the homology arms, e.g., the 5′ and 3′ homology arms, may each comprise about 1000 base pairs (bp) of sequence flanking the most distal gRNAs (e.g., 1000 bp of sequence on either side of the genetic disruption or target site).

In some embodiments, the template polynucleotide contains homology arms for targeting the endogenous Nur77 locus (exemplary nucleotide sequence of an endogenous human Nur77 set forth in SEQ ID NO:1; NCBI Reference Sequence: NM_001202233.1, encoding the amino acid sequence set forth in SEQ ID NO:2). In some embodiments, the genetic disruption of the Nur77 locus is introduced at or near the 3′ end of the coding region, e.g., at or near the final exon of the coding region the gene, including sequence immediately before a stop codon, e.g., within the final exon of the coding sequence, or within 500 bp of the stop codon (e.g., less than 500, 450, 400, 350, 300, 250, 200, 150, 100 or 50 bp). In some embodiments, the genetic disruption of the Nur77 locus is introduced at an early coding region in the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In some embodiments, the template polynucleotide comprises about 500 to 1000, e.g., 600 to 900 or 700 to 800, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequence, which is homologous to 500, 600, 700, 800, 900 or 1000 base pairs of sequence 5′ of the genetic disruption (e.g., at the Nur77 locus), the transgene, and about 500, 600, 700, 800, 900 or 1000 base pairs of 3′ homology arm sequence, which is homologous to 500, 600, 700, 800, 900 or 1000 base pairs of sequence 3′ of the genetic disruption (e.g., at the Nur77 locus).

In some embodiments, the location of the genetic disruption (e.g., target site) and the design of the template polynucleotide are selected such that upon introduction of the genetic disruption and targeted integration of the transgene, e.g., nucleic acid sequences encoding a reporter molecule, is in-frame with the endogenous gene, e.g., endogenous Nur77 gene. In some embodiments, the transgene, e.g., nucleic acid sequences encoding a reporter molecule, is integrated or is targeted for integration, in-frame, near the end of the final exon of the endogenous Nur77 gene, such that expression of the transgene is under operable control of the endogenous Nur77 transcriptional regulatory elements, while permitting the expression of the endogenous Nur77 polypeptide (in some cases, except for the final several amino acids at the C-terminal). In some embodiments, a ribosome skipping element/self-cleavage element, such as a 2A element, is placed upstream of the transgene coding sequence, such that the ribosome skipping element/self-cleavage element is placed in-frame with the endogenous gene. In some embodiments, the transgene, e.g., nucleic acid sequences encoding a reporter molecule, is integrated or is targeted for integration such that the endogenous Nur77 transcriptional regulatory elements control the expression of the endogenous Nur77 polypeptide-T2A-reporter molecule.

In some exemplary embodiments, the encoded reporter molecule is or comprises a fluorescent protein, a luciferase, a β-galactosidase, a chloramphenicol acetyltransferase (CAT), a β-glucuronidase (GUS), or a modified form thereof. In some embodiments, the fluorescent protein is or comprises a green fluorescent protein (GFP), enhanced green fluorescent protein (EGFP), a super-fold GFP, red fluorescent protein (RFP), cyan fluorescent protein (CFP), blue green fluorescent protein (BFP), enhanced blue fluorescent protein (EBFP), and yellow fluorescent protein (YFP), or a variant thereof, including species variants, monomeric variants, and codon-optimized and/or enhanced variants of the fluorescent proteins. In some embodiments, the encoded reporter molecule is a red fluorescent protein (RFP), such as tdTomato, mCherry, mStrawberry, AsRed2, DsRed or DsRed2. In some embodiments, the encoded reporter molecule is EGFP. For example, in some embodiments, the nucleic acid sequence encoding the reporter molecule comprises the sequence of nucleic acids set forth in SEQ ID NO: 10 or a sequence of nucleic acids that exhibits at least 85%, 86%, 87%, 88%, 89%, 90%, 91%, 92%, 93%, 94%, 95%, 96%, 97%, 98%, 99% or more sequence identity to any of SEQ ID NO: 10. In some embodiments, the encoded reporter molecule comprises the sequence of amino acids set forth in SEQ ID NO:11, or a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NO: 11

In some cases, the ribosome skipping element/self-cleavage element, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). This allows the inserted transgene to be controlled by the transcription of the endogenous promoter at the integration site, e.g., Nur77 promoter. Exemplary ribosome skipping element/self-cleavage element include 2A sequences from the foot-and-mouth disease virus, equine rhinitis A virus, Thosea asigna virus (T2A, e.g., SEQ ID NO: 6), and porcine teschovirus-1 as described in U.S. Patent Publication No. 20070116690. In some embodiments, exemplary ribosome skipping element/self-cleavage element includes a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NO: 6. In some embodiments, the template polynucleotide includes a T2A ribosome skipping element (sequence set forth in SEQ ID NO: 6 or 7) upstream of the transgene, e.g., nucleic acid sequences encoding a reporter molecule.

In some embodiments, the template polynucleotide comprises one or more mutations, e.g., silent mutations, that prevent the RNA-guided nuclease or DNA-binding nuclease fusion protein from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered. In some embodiments, the transgene contains one or more mutations, e.g., silent mutations that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered. In some embodiments, homology arm contained in the template polynucleotide includes silent mutations, to prevent the RNA-guided nuclease or DNA-binding nuclease fusion protein from recognizing and cleaving the template polynucleotide.

In some embodiments, an exemplary template polynucleotide contains a polynucleotides encoding a T2A ribosomal skip element (sequence set forth in SEQ ID NO:6, encoding polypeptide sequence set forth in SEQ ID NO: 7), the luciferase enzyme (FFLuc2 set forth in SEQ ID NO:8; encoding the polypeptide sequence set forth in SEQ ID NO: 9), and the eGFP fluorescent protein (sequence set forth in SEQ ID NO: 10; encoding polypeptide sequence set forth in SEQ ID NO:11), flanked on either side of the T2A, FFLuc2 and eGFP coding sequences by the 5′ homology arm (set forth in SEQ ID NO:12, containing 2 silent mutations compared to the corresponding Nur77 genomic sequence set forth in SEQ ID NO:1) and the 3′ homology arm (set forth in SEQ ID NO: 13), homologous to sequences surrounding the stop codon of the endogenous Nur77 gene. In some embodiments, the transgene, e.g., T2A-EGFP-FFLuc2 encoding sequences, can be targeted to be inserted in-frame with the endogenous Nur77 gene and prior to the stop codon. In some embodiments, an exemplary template polynucleotide for HDR includes a nucleic acid sequence set forth in SEQ ID: 5. In some embodiments, an exemplary target site sequence for introduction of the genetic disruption or cleavage comprises the nucleic acid sequence TCATTGACAAGATCTTCATG (SEQ ID NO:14) and/or GCCTGGGAACACGTGTGCA (SEQ ID NO:15).

### 2. Viral Vectors

In some embodiments, the methods involve contacting a cell composition, such as a reporter T cell composition as described in Section I.A.1, with a prepared test or reference viral vector (also referred to as a “viral vector composition”).

In some embodiments, the viral vector (e.g. retroviral vector, such as a lentiviral vector) contains a nucleic acid encoding a recombinant receptor, such as chimeric antigen receptor (CAR) or other antigen receptor, in a genome of the viral vector. The genome of the viral vector typically includes sequences in addition to the nucleic acid encoding the recombinant receptor. Such sequences may include sequences that allow the genome to be packaged into the virus particle and/or sequences that promote expression of a nucleic acid encoding a recombinant receptor, such as a CAR.

Viral vectors, including retroviral vectors, have become the dominant method for the introduction of genes into mammalian, e.g., human cells. Other sources of viral vectors include DNA viruses, poxviruses, herpes simplex virus I, adenoviruses and adeno-associated viruses. Methods for producing vectors, such as a vector containing a nucleic acid encoding a recombinant receptor, are well-known in the art. See, for example, Sambrook et al., 2001, Molecular Cloning: A Laboratory Manual, Cold Spring Harbor Laboratory, New York. In some of any such embodiments, the vector is a Adenovirus, Adeno-associated virus, or retrovirus, such as a lentivirus.

The provided viral vector particles contain a genome derived from a retroviral genome based vector, such as derived from a gammaretroviral or lentiviral genome based vector. Any of a large number of such suitable vector genomes are known ((see, e.g., Koste et al. (2014) Gene Therapy 2014 Apr. 3. doi: 10.1038/gt.2014.25; Carlens et al. (2000) Exp Hematol 28 (10): 1137-46; Alonso-Camino et al. (2013) Mol Ther Nucl Acids 2, e93; Park et al., Trends Biotechnol. 2011 Nov. 29 (11): 550-557; Pfeifer and Verma (2001) Annu. Rev. Genomics Hum. Genet., 2:177-211). In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

a. Retroviral Vectors

In some embodiments, the viral vector particles contain a genome derived from a retroviral genome based vector, such as derived from a lentiviral genome based vector. In some embodiments, the viral vector particle is a lentiviral vector particle. In some aspects of the provided viral vectors, a heterologous nucleic acid (e.g., polynucleotide) encoding a recombinant protein, such as an antigen receptor, such as a chimeric antigen receptor (CAR) or a transgenic T cell receptor (TCR), is contained and/or located between the 5′ LTR and 3′ LTR sequences of the vector genome. In some embodiments, the recombinant protein is an antigen receptor. In some embodiments, the recombinant protein is a T cell receptor (TCR). In some embodiments, the recombinant protein is a chimeric antigen receptor (CAR).

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72:9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Spl and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

b. Preparation of Retroviral Vectors

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g. antigen receptor, such as CAR, can be detected.

c. Nucleic Acid Encoding a Heterologous Protein

In some embodiments, the viral vector contains a nucleic acid (e.g., polynucleotide) that encodes a heterologous recombinant protein. In some embodiments, the heterologous recombinant protein or molecule is or includes a recombinant receptor, e.g., an antigen receptor, SB-transposons, e.g., for gene silencing, capsid-enclosed transposons, homologous double stranded nucleic acid, e.g., for genomic recombination or reporter genes (e.g., fluorescent proteins, such as GFP) or luciferase).

In some embodiments, the viral vector contains a nucleic acid (e.g., polynucleotide) that encodes a recombinant receptor and/or chimeric receptor, such as a heterologous receptor protein. The recombinant receptor, such as heterologous receptor, may include antigen receptors, such as functional non-TCR antigen receptors, including chimeric antigen receptors (CARs), and other antigen-binding receptors such as transgenic T cell receptors (TCRs). The receptors may also include other receptors, such as other chimeric receptors, such as receptors that bind to particular ligands and having transmembrane and/or intracellular signaling domains similar to those present in a CAR.

In any of such examples, the nucleic acid (e.g., polynucleotide) is inserted or located in a region of the viral vector, such as generally in a non-essential region of the viral genome. In some embodiments, the nucleic acid (e.g., polynucleotide) is inserted into the viral genome in the place of certain viral sequences to produce a virus that is replication defective.

In some embodiments, the encoded recombinant antigen receptor, e.g., CAR, is one that is capable of specifically binding to one or more ligand on a cell or disease to be targeted, such as a cancer, infectious disease, inflammatory or autoimmune disease, or other disease or condition, including those described herein for targeting with the provided methods and compositions.

In certain embodiments, an exemplary antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b, or CD30.

In some embodiments, the exemplary antigens are orphan tyrosine kinase receptor ROR1, tEGFR, Her2, Ll-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acetylcholine receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by and/or characteristic of or specific for HIV, HCV, HBV, HPV, and/or other pathogens and/or oncogenic versions thereof.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

Antigen receptors, including CARs and recombinant TCRs, and production and introduction thereof, in some embodiments include those described, for example, in international patent application publication numbers WO200014257, WO2013126726, WO2012/129514, WO2014031687, WO2013/166321, WO2013/071154, WO2013/123061, WO2015/168613, WO2016/030414, U.S. patent application publication numbers US2002131960, US2013287748, US20130149337, US20190389925, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3 (4): 388-398; Davila et al. (2013) PLOS ONE 8 (4): e61338; Turtle et al., Curr. Opin. Immunol., 2012 October; 24 (5): 633-39; Wu et al., Cancer, 2012 Mar. 18 (2): 160-75.

### 1) Chimeric Antigen Receptors

In some embodiments, the nucleic acid (e.g., polynucleotide) contained in a genome of the viral vector encodes a chimeric antigen receptor (CAR). The CAR is generally a genetically engineered receptor with an extracellular ligand binding domain, such as an extracellular portion containing an antibody or fragment thereof, linked to one or more intracellular signaling components. In some embodiments, the chimeric antigen receptor includes a transmembrane domain and/or intracellular domain linking the extracellular domain and the intracellular signaling domain. Such molecules typically mimic or approximate a signal through a natural antigen receptor and/or signal through such a receptor in combination with a costimulatory receptor.

In some embodiments, CARs are constructed with a specificity for a particular marker, such as a marker expressed in a particular cell type to be targeted by adoptive therapy, e.g., a cancer marker and/or any of the antigens described. Thus, the CAR typically includes one or more antigen-binding fragment, domain, or portion of an antibody, or one or more antibody variable domains, and/or antibody molecules. In some embodiments, the CAR includes an antigen-binding portion or portions of an antibody molecule, such as a variable heavy chain (VH) or antigen-binding portion thereof, or a single-chain antibody fragment (scFv) derived from the variable heavy (VH) and variable light (VL) chains of a monoclonal antibody (mAb).

In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain or region (also interchangeably called an intracellular signaling domain or region), such as a cytoplasmic (intracellular) region capable of inducing a primary activation signal in a T cell, for example, a cytoplasmic signaling domain or region of a T cell receptor (TCR) component (e.g., a cytoplasmic signaling domain or region of a zeta chain of a CD3-zeta (CD3ζ) chain or a functional variant or signaling portion thereof) and/or that comprises an immunoreceptor tyrosine-based activation motif (ITAM). In some embodiments, the CAR comprises an extracellular antigen-recognition domain that specifically binds to a target antigen and an intracellular signaling domain comprising an ITAM. In some embodiments, the intracellular signaling domain comprises an intracellular domain of a CD3-zeta (CD35) chain.

In some embodiments, the chimeric receptor further contains an extracellular ligand-binding domain that specifically binds to a ligand (e.g., antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3 (4): 388-398; Davila et al. (2013) PLOS ONE 8 (4): e61338; Turtle et al., Curr. Opin. Immunol., 2012 October; 24 (5): 633-39; Wu et al., Cancer, 2012 Mar. 18 (2): 160-75. In some aspects, the antigen receptors include a CAR as described in U.S. Pat. No. 7,446,190, and those described in International Patent Application Publication No. WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. No. 7,446,190, U.S. Pat. No. 8,389,282, Kochenderfer et al., 2013, Nature Reviews Clinical Oncology, 10, 267-276 (2013); Wang et al. (2012) J. Immunother. 35 (9): 689-701; and Brentjens et al., Sci Transl Med. 2013 5 (177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g., CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some embodiments, the antigen is associated with a disease or condition, such as cancer, an autoimmune disease or disorder, or an infectious disease. In some embodiments, the antigen receptor, e.g., CAR, specifically binds to a universal tag.

In some embodiments, the CAR contains an antibody or an antigen-binding fragment (e.g., scFv) that specifically recognizes an antigen, such as an intact antigen, expressed on the surface of a cell. In some embodiments, the target is an antigen of the recombinant receptor and thus, in some cases, the target-expressing cells are antigen-expressing cells

In some embodiments, the antigen (or a ligand) is a tumor antigen or cancer marker. In some embodiments, the antigen (or a ligand) the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes BCMA, CD20, CD19, CD22, ROR1, CD45, CD21, CD5, CD33, Igkappa, Iglambda, CD79a, CD79b, or CD30. In some embodiments, the antigen is or includes CD19. In some embodiments, the antigen is or includes BCMA.

In some embodiments, the antigen or antigen binding domain is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgG1 antibody raised against Nalm-1 and-16 cells expressing CD19 of human origin (Ling, N. R., et al. (1987). Leucocyte typing III. 302). In some embodiments, the FMC63 antibody comprises CDRH1 set forth in SEQ ID NOS: 19, CDRH2 set forth in SEQ ID NO: 20, and CDRH3 set forth in SEQ ID NO: 21 or SEQ ID NO:35, and CDRL1 set forth in SEQ ID NO: 16 and CDR L2 set forth in SEQ ID NO: 17 or 36 and CDR L3 set forth in SEQ ID NO: 18 or 37. In some embodiments, the FMC63 antibody comprises the heavy chain variable region (V_{H}) comprising the amino acid sequence of SEQ ID NO: 22 and the light chain variable region (V_{L}) comprising the amino acid sequence of SEQ ID NO: 23.

In some embodiments, the scFv comprises a variable light chain containing the CDRL1 sequence of SEQ ID NO:16, a CDRL2 sequence of SEQ ID NO: 17, and a CDRL3 sequence of SEQ ID NO: 18 and a variable heavy chain containing a CDRH1 sequence of SEQ ID NO: 19, a CDRH2 sequence of SEQ ID NO:20, and a CDRH3 sequence of SEQ ID NO: 21. In some embodiments, the scFv comprises a variable light chain containing the CDRL1 sequence of SEQ ID NO: 16, a CDRL2 sequence of SEQ ID NO:36, and a CDRL3 sequence of SEQ ID NO:37 and a variable heavy chain containing a CDRH1 sequence of SEQ ID NO:19, a CDRH2 sequence of SEQ ID NO:20, and a CDRH3 sequence of SEQ ID NO:35.

In some embodiments, the scFv comprises a variable heavy chain region set forth in SEQ ID NO:22 and a variable light chain region set forth in SEQ ID NO:23. In some embodiments, the variable heavy and variable light chains are connected by a linker. In some embodiments, the linker is set forth in SEQ ID NO:39. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:24 or a sequence that exhibits at least 85%, 86%, 87%, 88%, 89%, 90%, 91%, 92%, 93%, 94%, 95%, 96%, 97%, 98%, or 99% sequence identity to SEQ ID NO: 24. In some embodiments, the scFv comprises the sequence of amino acids set forth in SEQ ID NO: 24 or a sequence that exhibits at least 85%, 86%, 87%, 88%, 89%, 90%, 91%, 92%, 93%, 94%, 95%, 96%, 97%, 98%, or 99% sequence identity to SEQ ID NO:24.

In some embodiments the scFv is derived from SJ25C1. SJ25C1 is a mouse monoclonal IgG1 antibody raised against Nalm-1 and-16 cells expressing CD19 of human origin (Ling, N. R., et al. (1987). Leucocyte typing III. 302). In some embodiments, the SJ25C1 antibody comprises CDRH1, H2 and H3 set forth in SEQ ID NOS: 28-30, respectively, and CDRL1, L2 and L3 sequences set forth in SEQ ID NOS: 25-27, respectively. In some embodiments, the SJ25C1 antibody comprises the heavy chain variable region (V_{H}) comprising the amino acid sequence of SEQ ID NO: 31 and the light chain variable region (V_{L}) comprising the amino acid sequence of SEQ ID NO: 32.

In some embodiments, the scFv comprises a variable light chain containing the CDRL1 sequence of SEQ ID NO:25, a CDRL2 sequence of SEQ ID NO: 26, and a CDRL3 sequence of SEQ ID NO:27 and a variable heavy chain containing a CDRH1 sequence of SEQ ID NO: 28, a CDRH2 sequence of SEQ ID NO:29, and a CDRH3 sequence of SEQ ID NO: 30. In some embodiments, the scFv comprises a variable heavy chain region set forth in SEQ ID NO:31 and a variable light chain region set forth in SEQ ID NO:32. In some embodiments, the variable heavy and variable light chain are connected by a linker. In some embodiments, the linker is set forth in SEQ ID NO:33. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:34 or a sequence that exhibits at least 85%, 86%, 87%, 88%, 89%, 90%, 91%, 92%, 93%, 94%, 95%, 96%, 97%, 98%, or 99% sequence identity to SEQ ID NO:34.

In some embodiments, the antibody or an antigen-binding fragment (e.g. scFv or V_{H} domain) specifically recognizes an antigen, such as BCMA. In some embodiments, the antibody or antigen-binding fragment is derived from, or is a variant of, antibodies or antigen-binding fragment that specifically binds to BCMA.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human antibodies, and cells expressing such chimeric receptors have been previously described. See Carpenter et al., Clin Cancer Res., 2013, 19 (8): 2048-2060, WO 2016/090320, WO2016090327, WO2010104949A2 and WO2017173256. In some embodiments, the antigen or antigen binding domain is BCMA. In some embodiments, the scFv contains a VH and a VL derived from an antibody or an antibody fragment specific to BCMA. In some embodiments, the antibody or antibody fragment that binds BCMA is or contains a VH and a VL from an antibody or antibody fragment set forth in International Patent Applications, Publication Number WO 2016/090327 and WO 2016/090320.

In some embodiments, the antigen or antigen binding domain is GPRC5D. In some embodiments, the scFv contains a VH and a VL derived from an antibody or an antibody fragment specific to GPRC5D. In some embodiments, the antibody or antibody fragment that binds GPRC5D is or contains a VH and a VL from an antibody or antibody fragment set forth in International Patent Applications, Publication Number WO 2016/090329 and WO 2016/090312.

In some aspects, the CAR contains a ligand- (e.g., antigen-) binding domain that binds or recognizes, e.g., specifically binds, a universal tag or a universal epitope. In some aspects, the binding domain can bind a molecule, a tag, a polypeptide and/or an epitope that can be linked to a different binding molecule (e.g., antibody or antigen-binding fragment) that recognizes an antigen associated with a disease or disorder. Exemplary tag or epitope includes a dye (e.g., fluorescein isothiocyanate) or a biotin. In some aspects, a binding molecule (e.g., antibody or antigen-binding fragment) linked to a tag, that recognizes the antigen associated with a disease or disorder, e.g., tumor antigen, with an engineered cell expressing a CAR specific for the tag, to effect cytotoxicity or other effector function of the engineered cell. In some aspects, the specificity of the CAR to the antigen associated with a disease or disorder is provided by the tagged binding molecule (e.g., antibody), and different tagged binding molecule can be used to target different antigens. Exemplary CARs specific for a universal tag or a universal epitope include those described, e.g., in U.S. Pat. No. 9,233,125, WO 2016/030414, Urbanska et al., (2012) Cancer Res 72:1844-1852, and Tamada et al., (2012). Clin Cancer Res 18:6436-6445.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In some embodiments, the CAR contains a TCR-like antibody, such as an antibody or an antigen-binding fragment (e.g., scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e., MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated β2 microglobulin. Generally, MHC class II molecules are composed of two transmembrane glycoproteins, a and B, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g., US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen, or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g., US published application No. US20020150914, US2014/0294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH,F(ab′) 2; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the extracellular portion of the CAR, such as an antibody portion thereof, further includes a spacer, such as a spacer region between the antigen-recognition component, e.g. scFv, and a transmembrane domain. The spacer may be or include at least a portion of an immunoglobulin constant region or variant or modified version thereof, such as a hinge region, e.g., an IgG4 hinge region, and/or a CH1/CL and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. In some embodiments, the spacer has the sequence set forth in SEQ ID NO: 40, and is encoded by the sequence set forth in SEQ ID NO: 41. In some embodiments, the spacer has the sequence set forth in SEQ ID NO: 42. In some embodiments, the spacer has the sequence set forth in SEQ ID NO: 43.

In some embodiments, the constant region or portion is of IgD. In some embodiments, the spacer has the sequence set forth in SEQ ID NO: 44. In some embodiments, the spacer has a sequence of amino acids that exhibits at least or at least about 85%, 86%, 87%, 88%, 89%, 90%, 91%, 92%, 93%, 94%, 95%, 96%, 97%, 98%, or 99% or more sequence identity to any of SEQ ID NOS: 40, 42, 43, and 44.

In some embodiments, the spacer may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014/031687. In some embodiments, the spacer has the sequence set forth in SEQ ID NO: 40, and is encoded by the sequence set forth in SEQ ID NO: 41. In some embodiments, the spacer has the sequence set forth in SEQ ID NO: 42. In some embodiments, the spacer has the sequence set forth in SEQ ID NO: 43.

In some embodiments, the constant region or portion is of IgD. In some embodiments, the spacer has the sequence set forth in SEQ ID NO: 44. In some embodiments, the spacer has a sequence of amino acids that exhibits at least or at least about 85%, 86%, 87%, 88%, 89%, 90%, 91%, 92%, 93%, 94%, 95%, 96%, 97%, 98%, or 99% or more sequence identity to any of SEQ ID NOS: 40, 42, 43, and 44.

The extracellular ligand binding, such as antigen recognition domain, generally is linked to one or more intracellular signaling components, such as signaling components that mimic activation through an antigen receptor complex, such as a TCR complex, in the case of a CAR, and/or signal via another cell surface receptor. In some embodiments, a transmembrane domain links the extracellular ligand binding domain and intracellular signaling domains. In some embodiments, the antigen binding component (e.g., antibody) is linked to one or more transmembrane and intracellular signaling regions. In some embodiments, the CAR includes a transmembrane domain fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, or CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The recombinant receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor Y, CD8, CD4, CD25, or CD16. For example, in some aspects, the CAR or other chimeric receptor includes a chimeric molecule between CD3-zeta (CD3-2) or Fc receptor y and CD8, CD4, CD25, or CD16.

In some embodiments, upon ligation of the CAR or other chimeric receptor, the cytoplasmic domain and/or region or intracellular signaling domain and/or region of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by at least two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CD8, CD22, CD79a, CD79b, and CD66d. In certain embodiments, ITAM containing primary cytoplasmic signaling sequences include those derived from TCR or CD3 zeta, FcR gamma, or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, CD27, DAP10, and/or ICOS. In some aspects, the same CAR includes both the activating or signaling region and costimulatory components. In some embodiments, the intracellular signaling domain comprises an intracellular signaling domain of a T cell costimulatory molecule. In some embodiments, the T cell costimulatory molecule is selected from the group consisting of CD28 and 41BB.

In some embodiments, the activating domain is included within one CAR, whereas the costimulatory component is provided by another CAR recognizing another antigen. In some embodiments, the CARs include activating or stimulatory CARs, and costimulatory CARs, both expressed on the same cell (see WO2014/055668). In some aspects, the CAR is the stimulatory or activating CAR; in other aspects, it is the costimulatory CAR. In some embodiments, the cells further include inhibitory CARs (iCARs, see Fedorov et al., Sci. Transl. Medicine, 5 (215) (December 2013), such as a CAR recognizing a different antigen, whereby an activating signal delivered through a CAR recognizing a first antigen is diminished or inhibited by binding of the inhibitory CAR to its ligand, e.g., to reduce off-target effects.

In certain embodiments, the intracellular signaling domain comprises a CD28 transmembrane and signaling domain linked to a CD3 intracellular domain. In some embodiments, the intracellular signaling domain comprises a chimeric CD28 and CD137 co-stimulatory domains, linked to a CD3 intracellular domain.

In some embodiments, the intracellular signaling domain of the CD8+ cytotoxic T cells is the same as the intracellular signaling domain of the CD4+ helper T cells. In some embodiments, the intracellular signaling domain of the CD8+ cytotoxic T cells is different than the intracellular signaling domain of the CD4+ helper T cells.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the recombinant receptor(s), e.g., CAR, encoded by nucleic acid(s) (e.g., polynucleotide(s)) within the provided viral vectors further include one or more marker, e.g., for purposes of confirming transduction or engineering of the cell to express the receptor and/or selection and/or targeting of cells expressing molecule(s) encoded by the polynucleotide. In some aspects, such a marker may be encoded by a different nucleic acid or polynucleotide, which also may be introduced during the genetic engineering process, typically via the same method, e.g., transduction by any of the methods provided herein, e.g., via the same vector or type of vector.

In some aspects, the marker, e.g., transduction marker, is a protein and/or is a cell surface molecule. Exemplary markers are truncated variants of a naturally-occurring, e.g., endogenous markers, such as naturally-occurring cell surface molecules. In some aspects, the variants have reduced immunogenicity, reduced trafficking function, and/or reduced signaling function compared to the natural or endogenous cell surface molecule. In some embodiments, the marker is a truncated version of a cell surface receptor, such as truncated EGFR (tEGFR). In some aspects, the marker includes all or part (e.g., truncated form) of CD34, an NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A P2A, E2A and/or F2A. See, e.g., WO2014/031687.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular ligand-binding portion, such as an antigen-binding portion, such as an antibody or fragment thereof and in intracellular domain. In some embodiments, the antibody or fragment includes an scFv or a single-domain VH antibody and the intracellular domain contains an ITAM. In some aspects, the intracellular signaling domain includes a signaling domain of a zeta chain of a CD3-zeta (CD32) chain. In some embodiments, the chimeric antigen receptor includes a transmembrane domain linking and/or disposed between the extracellular domain and the intracellular signaling region or domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 45 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 45; in some embodiments, the transmembrane-domain containing portion of the recombinant receptor comprises the sequence of amino acids set forth in SEQ ID NO: 46 or a sequence of amino acids having at least or at least about 85%, 86%, 87%, 88%, 89%, 90%, 91%, 92%, 93%, 94%, 95%, 96%, 97%, 98%, or 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling region and/or domain can comprise the sequence of amino acids set forth in SEQ ID NO: 47 or 48 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 47 or 48. In some embodiments, the intracellular region and/or domain comprises an intracellular costimulatory signaling domain of 4-1BB or functional variant thereof, such as a 42-amino acid cytoplasmic domain of a human 4-1BB (Accession No. Q07011.1), or functional variant or portion thereof, such as the sequence of amino acids set forth in SEQ ID NO: 49 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 49.

In some embodiments, the intracellular signaling region and/or domain comprises a human CD3 chain, optionally a CD3 zeta stimulatory signaling domain or functional variant thereof, such as an 112 AA cytoplasmic domain of isoform 3 of human CD32 (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 50, 51, or 52 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 50, 51, or 52.

In some aspects, the spacer contains only a hinge region of an IgG, such as only a hinge of IgG4 or IgG1 such as the hinge only spacer set forth in SEQ ID NO: 40. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains, such as set forth in SEQ ID NO: 42. In some embodiments, the spacer is an Ig hinge, e.g., an IgG4 hinge, linked to a CH3 domain only, such as set forth in SEQ ID NO: 43. In some embodiments, the spacer is or comprises a glycine-serine rich sequence or other flexible linker such as known flexible linkers.

For example, in some embodiments, the CAR includes: an extracellular ligand-binding portion, such as an antigen-binding portion, such as an antibody or fragment thereof, including sdAbs and scFvs, that specifically binds an antigen, e.g., an antigen described herein; a spacer such as any of the Ig-hinge containing spacers; a transmembrane domain that is a portion of CD28 or a variant thereof; an intracellular signaling domain containing a signaling portion of CD28 or functional variant thereof; and a signaling portion of CD3 zeta signaling domain or functional variant thereof. In some embodiments, the CAR includes: an extracellular ligand-binding portion, such as an antigen-binding portion, such as an antibody or fragment thereof, including sdAbs and scFvs, that specifically binds an antigen, e.g., an antigen described herein; a spacer such as any of the Ig-hinge containing spacers; a transmembrane domain that is a portion of CD28 or a variant thereof; an intracellular signaling domain containing a signaling portion of 4-1BB or functional variant thereof; and a signaling portion of CD3 zeta signaling domain or functional variant thereof.

In some embodiments, such CAR constructs further includes a T2A ribosomal skip element and/or a tEGFR sequence, e.g., downstream of the CAR. In some embodiments, nucleic acid molecules encoding such CAR constructs further includes a sequence encoding a ribosomal skip element (e.g. T2A) followed by a sequence encoding a tEGFR sequence, e.g., downstream of the sequence encoding the CAR. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

### 3. Transduction

In any of the provided embodiments, the provided methods involve methods of introducing a viral vector into reporter cells by transduction. In some embodiments, transducing cells involves contacting, e.g., incubating, a viral vector particle with a cell composition comprising a plurality of the reporter cells.

In some embodiments, the cell composition (e.g., the transducing composition) is or has been incubated under stimulatory conditions prior to transducing the cells by incubating them with a viral vector particle. In some aspects, prior to the incubation, at least 60%, 65%, 70%, 75%, 80%, 85%, 90%, or 95% of the T cells of the cell composition are activated cells, e.g., express a surface marker selected from the group consisting of HLA-DR, CD25, CD69, CD71, CD40L, and 4-1BB; comprise intracellular expression of a cytokine selected from the group consisting of IL-2, IFN-gamma, and TNF-alpha, are in the G1 or later phase of the cell cycle, and/or are capable of proliferating. In some aspects, prior to the incubation, at least 10%, at least 20%, at least 30%, at least 40%, at least 50%, or at least 60% of the T cells of the cell composition are activated cells, e.g., express a surface marker selected from the group consisting of HLA-DR, CD25, CD69, CD71, CD40L, and 4-1BB; comprise intracellular expression of a cytokine selected from the group consisting of IL-2, IFN-gamma, and TNF-alpha, are in the G1 or later phase of the cell cycle, and/or are capable of proliferating.

In some embodiments, during or during at least a portion of the incubating and/or contacting, the cell composition can comprise one or more cytokines. In some embodiments, the cytokine is selected from IL-2, IL-7, or IL-15. In some embodiments, the cytokine is a recombinant cytokine. In some embodiments, the concentration of the cytokine in the cell composition, independently, is from or from about 1 IU/mL to 1500 IU/mL, such as from or from about 1 IU/mL to 100 IU/mL, 2 IU/mL to 50 IU/mL, 5 IU/mL to 10 IU/mL, 10 IU/mL to 500 IU/mL, 50 IU/mL to 250 IU/mL, 100 IU/mL to 200 IU/mL, 50 IU/mL to 1500 IU/mL, 100 IU/mL to 1000 IU/mL, or 200 IU/mL to 600 IU/mL. In some embodiments, the concentration of the cytokine in the cell composition, independently, is at least or at least about 1 IU/mL, 5 IU/mL, 10 IU/mL, 50 IU/mL, 100 IU/mL, 200 IU/mL, 500 IU/mL, 1000 IU/mL, or 1500 IU/mL. In certain aspects, an agent capable of activating an intracellular signaling domain of a TCR complex, such as an anti-CD3 and/or anti-CD28 antibody, also can be including during or during at least a portion of the incubating or subsequent to the incubating.

In some embodiments, during or during at least a portion of the incubating and/or contacting, the cell composition can comprises serum. In some embodiments, the serum is fetal bovine serum. In some embodiments, the serum is human serum. In some embodiments, the serum is present in the cell composition at a concentration from or from about 0.5% to 25% (v/v), 1.0% to 10% (v/v) or 2.5% to 5.0% (v/v), each inclusive. In some embodiments, the serum is present in the cell composition at a concentration that is at least or at least about 0.5% (v/v), 1.0% (v/v), 2.5% (v/v), 5% (v/v) or 10% (v/v).

In some embodiments, during or during at least a portion of the incubating and/or contacting, the cell composition is free and/or substantially free of serum. In some embodiments, during or during at least a portion of the incubating and/or contacting, the cell composition is incubated and/or contacted in the absence of serum. In particular embodiments, during or during at least a portion of the incubating and/or contacting, the cell composition is incubated and/or contacted in serum-free media. In some embodiments, the serum free media is a defined and/or well-defined cell culture media. In some embodiments, the serum free media is formulated to support growth, proliferation, health, homeostasis of cells of a certain cell type, such as immune cells, T cells, and/or CD4+ and CD8+ T cells.

In some embodiments, during or during at least a portion of the incubating and/or contacting, the cell composition comprises N-Acetylcysteine. In some embodiments, the concentration of N-Acetylcysteine in the cell composition is from or from about 0.4 mg/mL to 4 mg/mL, 0.8 mg/mL to 3.6 mg/mL, or 1.6 mg/mL to 2.4 mg/mL, each inclusive. In some embodiments, the concentration of N-Acetylcysteine in the cell composition is at least or at least about or about 0.4 mg/mL, 0.8 mg/mL, 1.2 mg/mL, 1.6 mg/mL, 2.0 mg/mL, 2.4 mg/mL, 2.8 mg/mL, 3.2 mg/mL, 3.6 mg/mL, or 4.0 mg/mL.

In some embodiments, a plurality of transductions are performed to produce a plurality of reporter T cells that have been introduced with the viral vector encoding the recombinant receptor. In some embodiments, a titrated amount of viral vector is added to each of a plurality of reporter cell compositions. In some embodiments, each titrated amount is a serial dilution of the viral vector lot (e.g. test viral vector lot). In some embodiments, the viral vector is diluted 2-fold to 10,000-fold or more, such as 2-fold to 5,000-fold, 2-fold to 2,000-fold. The particular range of dilutions can be empirically determined depending on the viral vector and encoded recombinant receptor being employed. For instance, the particular dilution range is one that results in a linear dose-response increase in detectable signal upon incubation with a recombinant receptor stimulating agent by a reference standard across the plurality of titrated amounts. In some embodiments, the particular range of serial dilutions is chosen to also include a lower asymptote of detectable signal and an upper asymptote of detectable signal that represent a minimum and a maximum responses, respectively.

In some embodiments, the concentration of cells of the cell composition is from or from about 1.0×10⁵ cells/mL to 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL, or 1×10⁸ cells/mL.

In some embodiments, the cell composition (e.g., the transducing composition) comprises at least at or about at least or about 25×10⁶ cells, 50×10⁶ cells, 75×10⁶ cells 100×10⁶ cells, 125×10⁶ cells, 150×10⁶ cells, 175×10⁶ cells, 200×10⁶ cells, 225×10⁶ cells, 250×10⁶ cells, 275×10⁶ cells, or 300×10⁶ cells. For example, in some embodiments, the cell composition (e.g., the transducing composition) comprises at least at or about at least or about 50×10⁶ cells, 100×10⁶ cells, or 200×10⁶ cells.

In some embodiments, the cell composition (e.g., the transducing composition) comprises at least at or about at least or about 25×10⁵ cells, 50×10⁵ cells, 75×10⁵ cells 100×10⁵ cells, 125×10⁵ cells, 150×10⁵ cells, 175×10⁵ cells, 200×10⁵ cells, 225×10⁵ cells, 250×10⁵ cells, 275×10⁵ cells, or 300×10⁵ cells. For example, in some embodiments, the cell composition (e.g., the transducing composition) comprises at least at or about at least or about 50×10⁵ cells, 100×10⁵ cells, or 200×10⁵ cells.

In some embodiments, the viral vector particles are provided at a certain ratio of copies of the viral vector particles per total number of cells as a Multiplicity of Infection (MOI). In some embodiments, MOI may refer to the ratio of agents (e.g. viral vector copies) to infection targets (e.g. cells). In some embodiments, the MOI is between 0.01-10 particles/cell in a population of reporter T cells. In some embodiments, the MOI is 0.001-10 particles/cell in a population of reporter T cells. In some embodiments, the MOI is at least 0.001, 0.01, 0.10, 1.0, or 10 particles/cell in a population of reporter T cells. In some embodiments, the MOI is 0.001, 0.01, 0.10, 1.0, or 10 particles/cell. in a population of reporter T cells

In some embodiments, the viral vector particles are provided at a certain ratio of copies of the viral vector particles or infectious units (IU) thereof, per total number of cells (IU/cell) in the cell composition of reporter T cells or total number of cells to be transduced (e.g., a certain ratio that is an MOI as described above). For example, in some embodiments, the viral vector particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL. In some embodiments, the titer of viral vector particles is at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL. In some embodiments, the titer of viral vector particles is at or about 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL, or any value between any of the foregoing.

In some embodiments, the method involves contacting or incubating, such as admixing, the cells with the viral vector particles. In some embodiments, the contacting or incubating is for 30 minutes to 72 hours, such as 30 minute to 48 hours, 30 minutes to 24 hours, or 1 hour to 24 hours, such as at least or about at least 30 minutes, 1 hour, 2 hours, 6 hours, 12 hours, 24 hours, or 36 hours or more.

In some embodiments, contacting or incubating is performed in solution. In some embodiments, the cells and viral particles are contacted in a volume of from or from about 0.5 mL to 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL, or 200 mL to 500 mL.

In some embodiments, contacting or incubating is performed in solution. In some embodiments, the cells and viral particles are contacted in a volume of from or from about 1 μL to 1 mL, such as from or from about 2 μL, 5 μL, 10 μL, 15 μL, 20 μL, 25 μL, 30 μL, 40 μL, 50 μL, 100 μL, 200 μL, 400 μL, 500 μL, or 1 mL, or any value between any of the foregoing.

In certain embodiments, at least a portion of the engineering, transduction, and/or transfection is conducted at a volume from about 5 mL to about 100 mL, such as from about 10 mL to about 50 mL, from about 15 mL to about 45 mL, from about 20 mL to about 40 mL, from about 25 mL to about 35 mL, or at or at about 30 mL.

In some embodiments, the incubation of the cells with the viral vector particles is carried out by contacting one or more cells of a composition with a nucleic acid molecule encoding the recombinant protein, e.g., recombinant receptor. In some embodiments, the contacting can be effected with centrifugation. Such methods include any of those as described in International Publication Number WO2016/073602. Exemplary centrifugal chambers include those produced and sold by Biosafe S A, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No. US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the incubation of the cells with the viral vector particles further comprises contacting the composition (e.g., stimulated composition) and/or viral vector particles with a transduction adjuvant. In some embodiments, the contacting the composition (e.g., stimulated composition) and/or the viral vector particles with a transduction adjuvant is carried out prior to, concomitant with, or after spinoculating the viral vector particles with the composition (e.g., stimulated composition).

In some embodiments, at least a portion of the incubation of the viral vector particle is carried out at or about 37° C.±2° C. For instance, in some embodiments, at least a portion of the incubation of the viral particle is carried out at or about 35-39° C. In some embodiments, the at least a portion of the incubation of the viral vector particle that is carried out at or about 37° C.±2° C. is carried out for no more than or no more than about 2 hours, 4 hours, 12 hours, 18 hours, 24 hours, 30 hours, 36 hours, 48 hours, 60 hours, or 72 hours. In some embodiments, the at least a portion of the incubation of the viral vector particle that is carried out at or about 37° C.±2° C. is carried out for or for about 24 hours.

In some embodiments, at least a portion of the incubation of the viral vector particle is carried out after the inoculation. In some embodiments, the at least a portion of the incubation of the viral vector particle that is carried out after the inoculation is carried out for no more than or no more than about 2 hours, 4 hours, 12 hours, 18 hours, 24 hours, 30 hours, 36 hours, 48 hours, 60 hours, or 72 hours. In some embodiments, the at least a portion of the incubation of the viral vector particle that is carried out after the inoculation is carried out for or for about 24 hours.

In some embodiments, the total duration of the incubation of the viral vector particle is for no more than 12 hours, 24 hours, 36 hours, 48 hours, or 72 hours.

In some embodiments, the incubation of the cells with the viral vector particles results in or produces an output composition comprising cells transduced with the viral vector particles, which is also referred to herein as a population of transduced cells. Accordingly, in some embodiments, the population of transduced cells comprises T cells transduced with the heterologous polynucleotide. In some embodiments, at least 20%, at least 25%, at least 30%, at least 35%, at least 40%, at least 45%, at least 50%, at least 55%, at least 60%, at least 65%, at least 70%, at least 75%, at least 80%, or at least 85% of the T cells in the population of transduced cells are transduced with the heterologous polynucleotide. In some embodiments, at least 55%, at least 60%, at least 65%, at least 70%, at least 75%, at least 80%, or at least 85% of the T cells in the population of transduced cells are transduced with the heterologous polynucleotide. In some embodiments, at least 95%, at least 96%, at least 97%, at least 98%, or at least 99% of the T cells transduced with the heterologous polynucleotide are CCR7+.

In some embodiments, the population of transduced cells comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% cells that express the recombinant protein. In some embodiments, the population of transduced cells comprises at least 55%, at least 60%, at least 65%, at least 70%, at least 75%, at least 80%, at least 85%, at least 90%, or at least 95% cells that express the recombinant protein.

In some embodiments, the method further comprises one or more additional steps. In some embodiments, the method further comprises recovering or isolating from the population of transduced cells the transduced cells produced by the method. In some embodiments, the recovering or isolating comprises selecting for expression of the recombinant protein (e.g., the CAR or TCR).

The percentage of T cells in the population of transduced cells that are transduced with the heterologous polynucleotide can be compared to the percentage of transduced T cells in other populations of transduced cells, e.g., the percentage of T cells in a plurality of populations of transduced cells that are transduced with a heterologous polynucleotide can be compared. In some embodiments, the maximum variability among the percentage of transduced T cells in the plurality of populations is less than 50%, less than 40%, less than 30%, less than 25%, less than 20%, less than 15%, less than 10%, or less than 5%, from the average percentage of transduction among the plurality. For example, a plurality of populations of transduced cells that include transduction percentages of 70%, 80%, and 90%, has a maximum variability of 12.5%. In some embodiments, the plurality of populations of transduced cells includes at least 5, at least 10, at least 20, at least 30, at least 40, at least 50, at least 60, at least 70, at least 80, at least 90, or at least 100 populations of transduced cells.

### B. Recombinant Receptor-Dependent Stimulation of Transduced Reporter Cells Via Binding Agents

The methods of assessing potency provided herein include means of stimulating the recombinant receptors (e.g., CARs, TCRs) of the reporter T cells introduced with the recombinant receptor. It is contemplated that any means suitable for stimulating the recombinant receptor that is also capable of being quantified may be used. In some embodiments, the means of stimulation of the recombinant receptor is achieved by a recombinant receptor stimulating agent able to bind to and stimulate an intracellular signal by the recombinant receptor to produce a detectable signal from the reporter, such as described in Section I-C. Exemplary recombinant receptor stimulating agents include antigens (e.g. purified or recombinant antigens) of the recombinant receptor, antibodies such as anti-idiotype antibodies, and antigen-expressing cells.

### 1. Surface Immobilized Agent

In particular embodiments, the recombinant receptor stimulating agent is composed of a binding molecule that is able to be bound by the recombinant receptor that is immobilized on a surface support. In provided embodiments, the binding molecule may be an antigen or a portion of an antigen of the recombinant receptor (e.g. extracellular portion of an antigen) or an antibody (e.g., an anti-idiotypic antibody) specific to the recombinant receptor. For instance, the binding molecule (e.g. antigen or binding portion thereof, or antibody) may be immobilized or bound to a surface support, such as a non-cell particle, wherein recombinant receptor-expressing cells (e.g. CAR-T cells) of the therapeutic composition, e.g. titrated amount of cells, are contacted with the surface support. In some embodiments, a particle described herein (e.g., bead particle) provides a solid support or matrix to which the binding molecule (e.g. an antigen or binding portion thereof, or an anti-idiotypic antibody), can be bound or attached in a manner that permits an interaction between the binding molecule and a cell, in particular binding between the binding molecule and a recombinant receptor, e.g., a CAR, expressed on the surface of the cell. In particular embodiments, the interaction between the conjugated or attached binding molecule and the cell mediates stimulation of the recombinant receptor, including one or more recombinant receptor-dependent activity such as activation, expansion, cytokine production, cytotoxicity activity or other activity as described, see e.g. Section I.C.

In certain embodiments, the surface support is a particle (e.g., a bead particle) to which the binding molecule (e.g. an antigen or binding portion thereof, or an anti-idiotypic antibody) is immobilized or attached. In some embodiments, the surface support is a solid support. In some examples, the solid support is a bead, and the antigen or portion is immobilized on the bead. In some embodiments, the solid support is the surface of a well or plate, e.g., a cell culture plate. In some embodiments, the surface support is a soluble oligomeric particle, and the antigen is immobilized on the surface of the soluble oligomeric particle. Examples of surface supports for immobilization or attachment of an agent (e.g. binding molecule) for recognition or binding to a recombinant receptor may be found in published International application WO 2019/027850, which is incorporated by reference for all purposes.

In particular embodiments, the surface support is a particle that may include a colloidal particle, a microsphere, nanoparticle, a bead, such as a magnetic bead, or the like. In some embodiments, the particles or beads are biocompatible, i.e. non-toxic. In certain embodiments the particles or beads are non-toxic to cultured cells, e.g., cultured T cells. In particular embodiments, the particles are monodisperse. In certain embodiments, “monodisperse” encompasses particles (e.g., bead particles) with size dispersions having a standard deviation of less than 5%, e.g., having less than a 5% standard deviation in diameter.

In some embodiments, the particle or bead is biocompatible, i.e., composed of a material that is suitable for biological use. In some embodiments, the particles, e.g., beads, are non-toxic to cultured cells, e.g., cultured T cells. In some embodiments, the particles, e.g., beads, may be any particles which are capable of attaching binding molecules in a manner that permits an interaction between the binding molecule and a cell. In certain embodiments, the particles, e.g., beads, may be any particles that can be modified, e.g., surface functionalized, to allow for the attachment of a binding molecule at the surface of the particle. In some embodiments, the particles, e.g., beads, are composed of glass, silica, polyesters of hydroxy carboxylic acids, polyanhydrides of dicarboxylic acids, or copolymers of hydroxy carboxylic acids and dicarboxylic acids. In some embodiments, the particles, e.g., beads, may be composed of or at least partially composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, particles, e.g., beads, can be quantum dots, or composed of quantum dots, such as quantum dot polystyrene particles, e.g., beads. Particles, e.g., beads, including mixtures of ester and anhydride bonds (e.g., copolymers of glycolic and sebacic acid) may also be employed. For example, particles, e.g., beads, may comprise materials including polyglycolic acid polymers (PGA), polylactic acid polymers (PLA), polysebacic acid polymers (PSA), poly(lactic-co-glycolic) acid copolymers (PLGA), [rho]poly(lactic-co-sebacic) acid copolymers (PLSA), poly(glycolic-co-sebacic) acid copolymers (PGSA), etc. Other polymers that particles, e.g., beads, may be composed of include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to binding molecules such as polypeptide antigen or antibodies.

In some embodiments, the particle is a bead that has a diameter of greater than 0.001 μm, greater than 0.01 μm, greater than 0.05 μm, greater than 0.1 μm, greater than 0.2 μm, greater than 0.3 μm, greater than 0.4 μm, greater than 0.5 μm, greater than 0.6 μm, greater than 0.7 μm, greater than 0.8 μm, greater than 0.9 μm, greater than 1 μm, greater than 2 μm, greater than 3 μm, greater than 4 μm, greater than 5 μm, greater than 6 μm, greater than 7 μm, greater than 8 μm, greater than 9 μm, greater than 10 μm, greater than 20 μm, greater than 30 μm, greater than 40 μm, greater than 50 μm, greater than 100 μm, greater than 500 μm, and/or greater than 1,000 μm. In some embodiments, the particles or beads have a diameter of between or between about 0.001 μm and 1,000 μm, 0.01 μm and 100 μm, 0.1 μm and 10, μm, 0.1 μm and 100 μm, 0.1 μm and 10 μm, 0.001 μm and 0.01 μm, 0.01 μm and 0.1 μm, 0.1 μm and 1 μm, 1 μm and 10 μm, 1 μm and 2 μm, 2 μm and 3 μm, 3 μm and 4 μm, 4 μm and 5 μm, 1 μm and 5 μm, and/or 5 μm and 10 μm, each inclusive. In certain embodiments, the particles or beads have a mean diameter of 1 μm and 10 μm, each inclusive. In certain embodiments, the particles, e.g., beads, have a diameter of or of about 1 μm. In particular embodiments, the particles, e.g., beads, have a mean diameter of or of about 2.8 μm. In some embodiments, the particles, e.g., beads, have a diameter of or of about 4.8 μm.

The particles (e.g., bead particles) used in the methods described herein can be produced or obtained commercially. Particles, e.g., beads, including methods of producing particles, e.g., beads, are well known in the art. See, for example, U.S. Pat. Nos. 6,074,884; 5,834,121; 5,395,688; 5,356,713; 5,318,797; 5,283,079; 5,232,782; 5,091,206; 4,774,265; 4,654,267; 4,554,088; 4,490,436; 4,452,773; U.S. Patent Application Publication No. 20100207051; and Sharpe, Pau T., Methods of Cell Separation, Elsevier, 1988. Commercially available particles, e.g., beads, (e.g., bead particles) include, but are not limited to, ProMag™ (PolySciences, Inc.); COMPEL™ (PolySciences, Inc.); BioMag® (PolySciences, Inc.), including BioMag® Plus (PolySciences, Inc.) and BioMag® Maxi (Bang Laboratories, Inc.); M-PVA (Cehmagen Biopolymer Technologie AG); SiMAG (Chemicell GmbH); beadMAG (Chemicell GmbH); MagaPhase® (Cortex Biochem); Dynabeads® (Invitrogen), including Dynabeads® M-280 Sheep Anti-rabbit IgG (Invitrogen), Dynabeads® FlowComp™ (e.g., Dynabeads® FlowCompTMHuman CD3, Invitrogen), Dynabeads® M-450 (e.g., Dynabeads® M-450 Tosylactivated, Invitrogen), Dynabeads® Untouched™ (e.g., Dynabeads® Untouched™ Human CD8 T Cells, Invitrogen), and Dynabeads® that bind, expand and/or activate T cells (e.g., Dynabeads® Human T-Activator CD3/CD28 for T Cell Expansion and Activation, Invitrogen); Estapor® M (Merk Chimie SAS); Estapor® EM (Merk Chimie SAS); MACSiBeads™ Particles (e.g., anti-biotin MACSiBead Particles, Miltenyi Biotec, catalog #130-091-147); Streptamer® Magnetic Beads (IBA BioTAGnology); Strep-Tactin® Magnetic Beads (IBA BioTAGnology); Sicastar®-M (Micormod Partikeltechnologie GmbH) Micromer®-M (Micromod Partikeltechnologie); MagneSil™ (Promega GmbH); MGP (Roche Applied Science Inc.); Pierce™ Protein G Magnetic Beads (Thermo Fisher Scientific Inc.); Pierce™ Protein A Magnetic Beads (Thermo Fisher Scientific Inc.); Pierce™ Protein A/G Magnetic Beads (Thermo Fisher Scientific Inc.); Pierce™ NHS-Activated Magnetic Beads (Thermo Fisher Scientific Inc.); Pierce™ Protein L Magnetic Beads (Thermo Fisher Scientific Inc.); Pierce™ Anti-HA Magnetic Beads (Thermo Fisher Scientific Inc.); Pierce™ Anti-c-Myc Magnetic Beads (Thermo Fisher Scientific Inc.); Pierce™ Glutathione Magnetic Beads (Thermo Fisher Scientific Inc.); Pierce™ Streptavidin Magnetic Beads (Thermo Fisher Scientific Inc.); MagnaBind™ Magnetic Beads (Thermo Fisher Scientific Inc.); Sera-Mag™ Magnetic Beads (Thermo Fisher Scientific Inc.); Anti-FLAG® M2 Magnetic Beads (Sigma-Aldrich); SPHERO™ Magnetic Particles (Spherotech Inc.); and HisPur™ Ni-NTA Magnetic Beads (Thermo Fisher Scientific Inc.).

In certain embodiments, the antigen or an extracellular domain portion thereof is bound to the particle (e.g. bead) via a covalent chemical bond. In particular embodiments, a reactive group or moiety of an amino acid of the antigen or extracellular domain portion thereof is conjugated directly to a reactive group or moiety on the surface of the particle by a direct chemical reaction. In certain embodiments, an amino acid carboxyl group (e.g., a C-terminal carboxyl group), hydroxyl, thiol, or amine group (such as an amino acid side chain group) of the antigen or extracellular binding portion thereof is conjugated directly to a hydroxyl or carboxyl group of a PLA or PGA polymer, a terminal amine or carboxyl group of a dendrimer, or a hydroxyl, carboxyl or phosphate group of a phospholipid on the surface of the particle by direct chemical reaction. In some embodiments, a conjugating moiety conjugates, e.g., covalently binds, to both the binding molecule and the particle, thereby linking them together.

In certain embodiments, the surface of the particle comprises chemical moieties and/or functional groups that allow attachment (e.g., covalent, non-covalent) of the binding molecule (e.g., polypeptide antigen or antibody). In particular embodiments, the particle surfaces contain exposed functional groups. Suitable surface exposed functional groups include, but are not limited to, carboxyl, amino, hydroxyl, sulfate groups, tosyl, epoxy, and chloromethyl groups. In some embodiments, the binding molecule is a polypeptide and is conjugated to the surface-exposed functional groups. In some embodiments, the surface exposed functional group must be activated, i.e., it must undergo a chemical reaction to yield an intermediate product capable of directly binding a polypeptide. For example, a carboxyl group of the polypeptide molecule may be activated with the agents described above to generate intermediate esters capable of directly binding to surface exposed amino groups of the particle. In other examples, free amine groups on the surface of a support surface (e.g. bead) may be covalently bound to antigen peptides and proteins, or antigen peptide or protein fusion proteins, using sulfosuccinimidyl (4-iodoacetyl)aminobenzoate (sulfo-SIAB) chemistry. In still other particular embodiments, a polypeptide binding molecule is covalently attached to the particle, e.g., a bead particle, at a surface exposed functional group that does not require activation by an agent prior to forming a covalent attachment. Examples of such functional groups include, but are not limited to, tosyl, epoxy, and chloromethyl groups.

In some embodiments, a non-covalent bond between a ligand bound to the antigen peptide or protein and an anti-ligand attached to the surface support (e.g. bead) may conjugate the antigen to the support (e.g. bead). In some embodiments, a biotin ligase recognition sequence tag may be joined to the C-terminus of an antigen peptide or protein, and this tag may be biotinylated by biotin ligase. The biotin may then serve as a ligand to non-covalently conjugate the antigen peptide or protein to avidin or streptavidin which is adsorbed or otherwise bound to the surface of the carrier as an anti-ligand. Alternatively, if the binding molecule (e.g. antigen) are fused to an immunoglobulin domain bearing an Fc region, as described herein, the Fc domain may act as a ligand, and protein A, either covalently or non-covalently bound to the surface of the surface support (e.g. bead), may serve as the anti-ligand to non-covalently conjugate the antigen peptide or protein to the carrier. Other means are well known in the art which may be employed to non-covalently conjugate binding molecules (e.g. antigen or anti-idiotypic antibody) to a surface support (e.g. beads (, including metal ion chelation techniques (e.g., using a poly-His tag at the C-terminus of the binding molecule, e.g. antigen, and a Ni-coated surface support), and these methods may be substituted for those described here.

In some embodiments, the binding molecule (e.g. antigen or anti-idiotypic antibody) is conjugated to the particle by a linker. In certain embodiments, the linkers can include, but are not limited to, a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particular coupling agents include N-succinimidyl-3-(2-pyridyldithio) propionate (SPDP) and N-succinimidyl-4-(2-pyridylthio) pentanoate (SPP) to provide for a disulfide linkage.

a. Target, e.g. Antigen

In some embodiments, the recombinant receptor stimulating agent is or includes a target, e.g., an antigen, a recombinant antigen, or fragment thereof. In some embodiments, the target is an antigen of the recombinant receptor. In some embodiments, the recombinant receptor stimulating agent is or includes an antigen, e.g., a recombinant antigen or fragment thereof.

For instance, the recombinant receptor stimulating agent may be target, such as an antigen, that is immobilized or bound to a surface support, such as a microwell plate, a solid particle (e.g. bead) or an oligomeric particle, e.g. as described above. In some embodiments, the target, e.g. antigen, is a polypeptide, or a variant or fragment of a polypeptide that is expressed on the surface of a cell that is associated with a disease, for example, a cancer cell and/or a tumor cell. It is understood that the target is any molecule that is recognized or bound by an extracellular domain of the recombinant receptor. In some embodiments, the target is an antibody that is recognized or bound by an extracellular domain of the recombinant receptor. In some embodiments, the target is a an antigen and it is understood that the antigen is an antigen that is recognized or bound by an extracellular domain of the recombinant receptor. A skilled artisan can determine the target, suc has an antigen, and format of the target or antigen (e.g. cell expressed or immobilized on a solid surface) sufficient to stimulate the recombinant receptor.

In some embodiments, the target is an antigen recognized by the extracellular domain of the recombinant receptor. In some embodiments, the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or comprises a portion of a polypeptide antigen that is recognized by or bound by a recombinant receptor, e.g. a CAR. In particular embodiments, the portion of an antigen is a region that contains an epitope that is recognized by or bound by a recombinant receptor, e.g. a CAR. In certain embodiments, the portion of the polypeptide antigen contains, about, or contains at least 10, 15, 20, 25, 30, 35, 40, 50, 55, 60, 65, 70, 75, 80, 85, 90, 95, 100, 110, 120, 130, 140, 150, 160, 170, 180, 190, 200, 250, 300, 400, or 500 amino acids, in some cases contiguous amino acids, of the polypeptide that is recognized by or bound by a recombinant receptor and or a CAR. In certain embodiments, the polypeptide portion comprises an amino acid sequence of the epitope that is recognized by the recombinant receptor and/or CAR.

In certain embodiments, the antigen or portions is a polypeptide variant that contains, contains about, or contains at least 50%, 55%, 60%, 65%, 70%, 75%, 80%, 85%, 90%, 95%, 97%, 98%, 99%, or 99.5% amino acid sequence identity to a polypeptide that is bound by and/or recognized by recombinant receptor and/or CAR.

In certain embodiments, the extracellular domain of the recombinant receptor (e.g. CAR) is specific for or binds to BCMA and the antigen is BCMA or is an extracellular domain portion of BCMA. In some embodiments, the BCMA polypeptide is a mammalian BCMA polypeptide. In particular embodiments, the BCMA polypeptide is a human BCMA polypeptide. In some embodiments, the BCMA antigen is or comprises an extracellular domain of BCMA or a portion thereof comprising an epitope recognized by an antigen receptor, e.g. CAR. In certain embodiments, the BCMA antigen is or comprises a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 53 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 53. In some embodiments, the BCMA antigen is or includes the sequence set forth in SEQ ID NO: 53 or a portion thereof that is or contains an epitope recognized by an antigen receptor, e.g. CAR.

In certain embodiments, the extracellular domain of the recombinant receptor (e.g. CAR) is specific for or binds to ROR1 and the antigen is ROR1 or is an extracellular domain portion of ROR1. In certain embodiments, the ROR1 polypeptide is mammalian. In particular embodiments, the ROR1 polypeptide is human. In some embodiments, the antigen is an extracellular domain of ROR1 or a portion thereof comprising an epitope recognized by an antigen receptor, e.g. CAR. In some embodiments, the antigen is a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 49 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 49. In some embodiments, the ROR1 antigen comprises the sequence set forth in SEQ ID NO: 49 or a portion thereof comprising an epitope recognized by an antigen receptor, e.g. CAR.

In certain embodiments, the extracellular domain of the recombinant receptor (e.g. CAR) is specific for or binds to CD22 and the antigen is CD22 or is an extracellular domain portion of CD22. In certain embodiments, the CD22 polypeptide is mammalian. In particular embodiments, the CD22 polypeptide is human. In some embodiments, the antigen is an extracellular domain of CD22 or a portion thereof comprising an epitope recognized by an antigen receptor, e.g. CAR. In some embodiments, the antigen is a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 54 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 54. In some embodiments, the CD22 antigen comprises the sequence set forth in SEQ ID NO: 54 or a portion thereof comprising an epitope recognized by an antigen receptor, e.g. CAR.

In certain embodiments, the extracellular domain of the recombinant receptor (e.g. CAR) is specific for or binds to CD19 and the antigen is CD19 or is an extracellular domain portion of CD19. In certain embodiments, the CD19 polypeptide is mammalian. In particular embodiments, the CD19 polypeptide is human. In some embodiments, the antigen is an extracellular domain of CD19 or a portion thereof comprising an epitope recognized by an antigen receptor, e.g. CAR. In some embodiments, the antigen is a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 45 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 45. In some embodiments, the CD19 antigen comprises the sequence set forth in SEQ ID NO: 45 or a portion thereof comprising an epitope recognized by an antigen receptor, e.g. CAR.

In some embodiments, the antigen or portion thereof may be formatted as a multimer, e.g. a dimer, comprising two or more polypeptide antigens, or portion or variant thereof, that is recognized and/or bound by a recombinant receptor, such as an antigen receptor (e.g. a CAR). In some embodiments, the polypeptide antigen, or portion thereof, are identical. In certain embodiments, the polypeptide antigen is linked, directly or indirectly, to a region or domain, e.g. a multimerization domain, that promotes or stabilizes interaction between two or more polypeptide antigens via complementary interactions between the domains or regions. In some embodiments, providing the polypeptide antigen as a multimer, e.g. dimer, provides for a multivalent interaction between the antigen or extracellular domain portion thereof and the antigen-binding domain of the antigen receptor, e.g. CAR, which, in some aspects, can increase the avidity of the interaction. In some embodiment, an increased avidity may favor stimulatory or agonist activity of antigen receptor, e.g. CAR, by the antigen or extracellular domain portion thereof conjugated to the bead.

In some embodiments, a polypeptide is joined directly or indirectly to a multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof. In particular embodiments, the polypeptide antigen is linked, directly or indirectly, to an Fc domain. In some embodiments, the polypeptide is a fusion polypeptide comprising the polypeptide antigen or portion thereof and the Fc domain.

In particular embodiments, an antigen or extracellular domain portion thereof is a fusion polypeptide that comprises an Fc domain. In some embodiments, the Fc domain is composed of the second and third constant domains (i.e., CH2 and CH3 domains) of the heavy chain of a IgG, IgA or IgD isotype, e.g. CH2 or CH3 of IgG, IgA and IgD isotypes. In some embodiments, the Fc domain is composed of three heavy chain constant domain (i.e., CH2, CH3, and CH4 domains) of an IgM or IgE isotype. In some embodiments, the Fc domain may further include a hinge sequence or portion thereof. In certain aspects, the Fc domain contains part or all of a hinge domain of an immunoglobulin molecule plus a CH2 and a CH3 domain. In some cases, the Fc domain can form a dimer of two polypeptide chains joined by one or more disulfide bonds. In some embodiments, the Fc domain is derived from an immunoglobulin (e.g., IgG, IgA, IgM, or IgE) of a suitable mammal (e.g., human, mouse, rat, goat, sheep, or monkey). In some embodiments, the Fc domain comprises CH2 and CH3 domains of IgG. In certain embodiments, the Fc domain is fused to the C-terminal of the polypeptide antigen. In particular embodiments, the Fc domain is fused to the N-terminal of the polypeptide antigen.

In some embodiments, the Fc domain is an IgG Fc domain, or a portion or variant thereof. In some embodiments, the Fc domain is a human IgG Fc domain, or a portion or a variant thereof, that comprises an amino acid sequence set forth in SEQ ID NO: 46 or an amino acid sequence that is at least 70%, 75%, 80%, 85%, 86%, 87%, 88%, 89%, 90%, 91%, 92%, 93%, 94%, 95%, 96%, 97%, 98%, 99% sequence identity to the sequence set forth in SEQ ID NO: 46. In particular embodiments, the Fc domain is a wild-type human IgG Fc domain, or a portion or variant thereof. In particular embodiments, the Fc domain is a variant of the wild-type human IgG1 Fc domain.

In some embodiments, the fusion polypeptide comprises a variant Fc domain. In certain embodiments, the variant human IgG Fc domain contains a mutation, e.g., a substitution, deletion, or insertion, that reduces, decreases, and/or diminishes pairing between the Fc domain and a light chain. In some embodiments, the variant human IgG Fc domain contains a mutation that reduces the binding affinity between the Fc domain and an Fc Receptor. In particular embodiments, the variant human IgG Fc domain contains a mutation that reduces, decreases, and/or diminishes the interactions, or the probability or likelihood of an interaction, between the Fc domain and an Fc Receptor. In some embodiments, the variant human IgG Fc domain contains a mutation that reduces the binding affinity between the Fc domain and a protein of the complement system. In particular embodiments, the variant human IgG Fc domain contains a mutation that reduces, decreases, and/or diminishes the interactions, or the probability or likelihood of an interaction, between the Fc domain and a protein of the complement system.

In some embodiments, the antigen or portion thereof is linked to a variant human IgG1 Fc domain. In some embodiments, the variant human IgG Fc domain contains a cystine to serine substitution in the hinge region of the Fc domain. In some embodiments, the variant human IgG Fc domain contains a leucine to alanine substitution in the hinge region of the Fc domain. In particular embodiments, the variant human IgG Fc domain contains a glycine to alanine substitution in the hinge region. In certain embodiments, the variant human IgG Fc domain contains an alanine to a serine substitution in the CH2 region of the Fc domain. In some embodiments, the variant human IgG Fc domain comprises a proline to serine substitution in the CH2 region of the Fc domain. In some embodiments, the variant human IgG Fc domain comprises an amino acid sequence as set forth by SEQ ID NO: 47.

In some embodiments, the antigen or extracellular domain portion thereof is provided as a fusion polypeptide comprising an Fc domain, wherein the Fc domain is present at the C-terminus of the fusion polypeptide.

In some embodiments, the antigen and the multimerization domain, such as Fc domain, are connected by a linker, such as an amino acid linker. In certain embodiments, the antigen is fused to the N-terminus of an amino acid linker, and the multimerization domain, such as Fc domain, is fused to the C-terminus of the linker. Although amino acid linkers can be any length and contain any combination of amino acids, the linker length may be relatively short (e.g., ten or fewer amino acids) to reduce interactions between the linked domains. The amino acid composition of the linker also may be adjusted to reduce the number of amino acids with bulky side chains or amino acids likely to introduce secondary structure. Suitable amino acid linkers include, but are not limited to, those up to 3, 4, 5, 6, 7, 10, 15, 20, or 25 amino acids in length. Representative amino acid linker sequences include GGGGS (SEQ ID NO: 52), and linkers comprising 2, 3, 4, or 5 copies of GGGGS (SEQ ID NO: 22).

In some embodiments, the antigen is provided as an extracellular domain of BCMA, e.g. human BCMA, fused to an Fc domain (BCMA-Fc). In particular embodiments, the BCMA-Fc antigen contains all or a portion of the amino acid sequence set forth in SEQ ID NO: 48 or a sequence of amino acids that exhibits at least 70%, 75%, 80%, 85%, 86%, 87%, 88%, 89%, 90%, 91%, 92%, 93%, 94%, 95%, 96%, 97%, 98%, 99% to SEQ ID NO: 48, and that comprises an epitope recognize by an antigen receptor, e.g. CAR.

In some embodiments, the antigen is provided as an extracellular domain of ROR1, e.g. human ROR1, fused to an Fc domain (ROR1-Fc). In certain embodiments, the ROR-1-Fc antigen contains all or a portion of the amino acid sequence set forth in SEQ ID NO: 20 or a sequence of amino acids that exhibits at least 70%, 75%, 80%, 85%, 86%, 87%, 88%, 89%, 90%, 91%, 92%, 93%, 94%, 95%, 96%, 97%, 98%, 99% to SEQ ID NO: 50 and that comprises an epitope recognize by an antigen receptor, e.g. CAR.

In particular embodiments, The antigen is provided as an extracellular domain of CD22, e.g. human CD22, fused to an Fc domain (e.g. CD22-Fc). In certain embodiments, the CD22-Fc antigen contains all or a portion of the amino acid sequence set forth in SEQ ID NO: 51 or a sequence of amino acids that exhibits at least 70%, 75%, 80%, 85%, 86%, 87%, 88%, 89%, 90%, 91%, 92%, 93%, 94%, 95%, 96%, 97%, 98%, 99% to SEQ ID NO: 51 and that comprises an epitope recognize by an antigen receptor, e.g. CAR.

In some embodiments, an Fc fusion of an antigen or an extracellular binding domain thereof is linked or attached to the surface support as a dimer formed by two Fc fusion polypeptides containing the polypeptide antigen or portion thereof an Fc domain. In some embodiments, the resulting polypeptide antigen-Fc fusion protein, e.g. BCMA-Fc, ROR1-Fc, CD22-Fc, or CD19-Fc, can be expressed in host cells, e.g. transformed with the expression vectors, whereby assembly between Fc domains can occurs by interchain disulfide bonds formed between the Fc moieties to yield a dimeric, such as divalent, polypeptide antigen fusion protein. In some embodiments, the host cell is a mammalian cell line. Exemplary of mammalian cells for recombinant expression of proteins include HEK293 cells or CHO cells or derivatives thereof. In some aspects, the nucleic acid encoding the Fc fusion protein further includes a signal peptide for secretion from the cell. In an exemplary embodiment, the signal peptide is CD33 (e.g. set forth in SEQ ID NO: 44).

In some embodiments, the cell of the therapeutic cell composition expresses a CAR that binds to or recognizes a universal tag that can be fused to an antibody or a fragment or variant thereof. In particular embodiments, cells expressing such CARs are able to specifically recognize and kill target cells, for example tumor cells, that have been bound by antibodies that have been fused with the universal tag. One example includes, but is not limited to, anti-FITC CAR expressing T cells that can bind to and/or recognize various human cancer cells when those cells are bound by cancer-reactive FITC-labeled antibodies. Thus, in some embodiments, the same CAR that binds to the universal tag is useful for the treatment of different cancers, provided there are available antibodies that recognize antigens associated with the cancers that contain the universal tag. In particular embodiments, a particle (e.g., a bead particle) comprises a surface exposed binding molecule that comprises universal tag binding molecule that is able to be bound by or recognized by recombinant receptor, e.g. CAR. In certain embodiments, the binding molecule is a universal tag or a portion thereof bound or recognized by the antigen receptor, e.g. CAR. Particular embodiments contemplate that any polypeptide domain that can be fused to an antibody, or an antigen binding fragment or variant thereof, that does not prevent the antibody from binding to its respective target is suitable for use as a universal tag. In some embodiments, a particle is bound to a binding molecule that comprises a universal tag, or a portion thereof, selected from the group consisting of: FITC, streptavidin, biotin, histidine, dinitrophenol, peridinin chlorophyll protein complex, green fluorescent protein, PE, HRP, palmitoylation, nitrosylation, alkalanine phosphatase, glucose oxidase, and maltose binding protein.

b. Antibodies

In some aspects, the binding molecule is an antibody (e.g., an anti-idiotype antibody) or antigen-binding fragment thereof (“anti-IDs”) that specifically recognizes a recombinant receptor, for example a recombinant receptor, e.g., CAR, as described in Section III. In particular, an anti-idiotype antibody targets the antigen binding site of another antibody, such as the scFv of the extracellular antigen binding domain of a CAR. In some embodiments, the anti-ID is able to bind to the recombinant receptor to stimulate a recombinant receptor-dependent activity. Exemplary anti-idiotype antibodies against antigen-specific CARs are known. These include, but are not limited to, anti-idiotypic antibodies directed against a CD22-directed CAR, see e.g. PCT Publication No. WO2013188864; CD19-directed CAR, see e.g. PCT Publication No. WO 2018/023100; a GPRC5D-directed CAR, see e.g. PCT Application No. PCT/US2020/063497; and a BCMA-directed CAR, see e.g. PCT Application No. PCT/US2020/063492. The anti-idiotypic antibody can be immobilized or attached to a surface support (e.g. bead) as described above for use as a recombinant receptor stimulating agent against cells expressing the recombinant receptor (e.g. CAR) targeted by the anti-idiotypic antibody.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The term “anti-idiotype antibody” refers to an antibody, including antigen-binding fragments thereof, that specifically recognizes, is specifically targeted to, and/or specifically binds to an idiotope of an antibody, such as an antigen-binding fragment. The idiotopes of an antibody may include, but are not necessarily limited to, residues within one or more of complementarity determining region(s) (CDRs) of the antibody, variable regions of the antibody, and/or partial portions or portions of such variable regions and/or of such CDRs, and/or any combination of the foregoing. The CDR may be one or more selected from the group consisting of CDR-H1, CDR-H2, CDR-H3, CDR-L1, CDR-L2, and CDR-L3. The variable regions of the antibody may be heavy chain variable regions, light chain variable regions, or a combination of the heavy chain variable regions and the light chain variable regions. The partial fragments or portions of the heavy chain variable regions and/or the light chain variable regions of the antibody may be fragments including 2 or more, 5 or more, or 10 or more contiguous amino acids, for example, from about 2 to about 100, from about 5 to about 100, from about 10 to about 100, from about 2 to about 50, from about 5 to about 50, or from about 10 to about 50 contiguous amino acids within the heavy chain variable regions or the light chain variable regions of the antibody; the idiotope may include multiple non-contiguous stretches of amino acids. The partial fragments of the heavy chain variable regions and the light chain variable regions of the antibody may be fragments including 2 or more, 5 or more, or 10 or more contiguous amino acids, for example, from about 2 to about 100, from about 5 to about 100, from about 10 to about 100, from about 2 to about 50, from about 5 to about 50, or from about 10 to about 50 contiguous amino acids within the variable regions, and in some embodiments contain one or more CDRs or CDR fragments. The CDR fragments may be consecutive or non-consecutive 2 or more, or 5 or more amino acids within the CDR. Therefore, the idiotopes of the antibody may be from about 2 to about 100, from about 5 to about 100, from about 10 to about 100, from about 2 to about 50, from about 5 to about 50, or from about 10 to about 50 contiguous amino acids containing one or more CDR or one or more CDR fragments within the heavy chain variable regions or the light chain variable regions of the antibody. In another embodiment, the idiotopes may be a single amino acid which is located at the variable regions of the antibody, for example, CDR sites.

In some embodiments, the idiotope is any single antigenic determinant or epitope within the variable portion of an antibody. In some cases it can overlap the actual antigen-binding site of the antibody, and in some cases it may comprise variable region sequences outside of the antigen-binding site of the antibody. The set of individual idiotopes of an antibody is in some embodiments referred to as the “idiotype” of such antibody.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), A1-Lazikani et al., (1997) JMB 273,927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 1, below, lists exemplary position boundaries of CDR-L1, CDR-L2, CDR-L3 and CDR-H1, CDR-H2, CDR-H3 as identified by Kabat, Chothia, and Contact schemes, respectively. For CDR-H1, residue numbering is listed using both the Kabat and Chothia numbering schemes. FRs are located between CDRs, for example, with FR-L1 located between CDR-L1 and CDR-L2, and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1

| CDR | Kabat | Chothia | Contact |
|---|---|---|---|
| CDR-L1 | L24-L34 | L24-L34 | L30-L36 |
| CDR-L2 | L50-L56 | L50-L56 | L46-L55 |
| CDR-L3 | L89-L97 | L89-L97 | L89-L96 |
| CDR-H1 | H31-H35B | H26-H32 . . . 34 | H30-H35B |
| (Kabat Numbering¹) | | | |
| CDR-H1 | H31-H35 | H26-H32 | H30-H35 |
| (Chothia Numbering²) | | | |
| CDR-H2 | H50-H65 | H52-H56 | H47-H58 |
| CDR-H3 | H95-H102 | H95-H102 | H93-H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or V_{L} amino acid sequence, it is understood that such a CDR has a sequence of the corresponding CDR (e.g., CDR-H3) within the variable region, as defined by any of the aforementioned schemes. In some embodiments, specified CDR sequences are specified.

Likewise, unless otherwise specified, a FR or individual specified FR(s) (e.g., FR-H1, FR-H2), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, or Contact method. In other cases, the particular amino acid sequence of a CDR or FR is given.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In particular embodiments, the antibodies are single-chain antibody fragments comprising a variable heavy chain region and/or a variable light chain region, such as scFvs.

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all framework regions (FRs) amino acid residues are derived from human FRs. In some embodiments, the humanized forms of a non-human antibody, e.g., a murine antibody, are chimeric antibodies that contain minimal sequences derived from non-human immunoglobulin. In certain embodiments, the humanized antibodies are antibodies from non-human species having one or more complementarily determining regions (CDRs) from the non-human species and a framework region (FR) from a human immunoglobulin molecule. In some embodiments, a humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity. (See, e.g., Queen, U.S. Pat. No. 5,585,089 and Winter, U.S. Pat. No. 5,225,539.) Such chimeric and humanized monoclonal antibodies can be produced by recombinant DNA techniques known in the art.

In certain embodiments, a humanized antibody is a human immunoglobulin (recipient antibody) in which residues from a heavy chain variable region of the recipient are replaced by residues from a heavy chain variable region of a non-human species (donor antibody) such as mouse, rat, rabbit, or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. In some embodiments, a nucleic acid sequences encoding human variable heavy chains and variable light chains are altered to replace one or more CDR sequences of the human (acceptor) sequence by sequence encoding the respective CDR in the nonhuman antibody sequence (donor sequence). In some embodiments, the human acceptor sequence may comprise FR derived from different genes. In particular embodiments, a humanized antibody will contain substantially all of at least one, and typically two, variable domains, in which all or substantially all of the hypervariable loops correspond to those of a non-human immunoglobulin, and all or substantially all of the FRs are those of a human immunoglobulin sequence. In some embodiments, the humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, incorporated by reference herein. In some embodiments, provided herein are humanized anti-idiotype antibodies.

In particular embodiments, an antibody, e.g., an anti-idiotype antibody, is humanized. In certain embodiments, the antibody is humanized by any suitable known means. For example, in some embodiments, a humanized antibody can have one or more amino acid residues introduced into it from a source which is non-human. These non-human amino acid residues are often referred to as “import” residues, which are typically taken from an “import” variable domain. In particular embodiments, humanization can be essentially performed by following the method of Winter and co-workers (Jones et al. (1986) Nature 321:522-525; Riechmann et al. (1988) Nature 332:323-327; Verhoeyen et al. (1988) Science 239:1534-1536), such as by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In certain embodiments, the humanized antibody is a human antibody in which some hypervariable region residues and possibly some FR residues are substituted by residues from analogous sites in rodent antibodies.

Sequences encoding full length antibodies can be subsequently obtained by joining the rendered variable heavy and variable light chain sequences to human constant heavy chain and constant light chain regions. Suitable human constant light chain sequences include kappa and lambda constant light chain sequences. Suitable human constant heavy chain sequences include IgG1, IgG2 and sequences encoding IgG1 mutants which have rendered immune-stimulating properties. Such mutants may have a reduced ability to activate complement and/or antibody dependent cellular cytotoxicity and are described in U.S. Pat. No. 5,624,821; WO 99/58572, U.S. Pat. No. 6,737,056. A suitable constant heavy chain also includes an IgG1 comprising the substitutions E233P, L234V, L235A, A327G, A330S, P331S and a deletion of residue 236. In another embodiment, the full length antibody comprises an IgA, IgD, IgE, IgM, IgY or IgW sequence.

Suitable human donor sequences can be determined by sequence comparison of the peptide sequences encoded by the mouse donor sequences to a group of human sequences, preferably to sequences encoded by human germ line immunoglobulin genes or mature antibody genes. A human sequence with a high sequence homology, preferably with the highest homology determined may serve as the acceptor sequence to for the humanization process.

In addition to the exchange of human CDRs for mouse CDRs, further manipulations in the human donor sequence may be carried out to obtain a sequence encoding a humanized antibody with optimized properties (such as affinity of the antigen).

Furthermore the altered human acceptor antibody variable domain sequences may also be rendered to encode one or more amino acids (according to the Kabat numbering system) of position 4, 35, 38, 43, 44, 46, 58, 62, 64, 65, 66, 67, 68, 69, 73, 85, 98 of the light variable region and 2, 4, 36, 39, 43, 45, 69, 70, 74, 75, 76, 78, 92 of the heavy variable region corresponding to the non-human donor sequence (Carter and Presta, U.S. Pat. No. 6,407,213)

In particular embodiments, it is generally desirable that antibodies be humanized with retention of high affinity for the antigen and other favorable biological properties. To achieve this goal, in some embodiments, the humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and imported sequences so that the desired antibody characteristic, such as increased affinity for the target antigen(s), is achieved. In general, the hypervariable region residues are directly and most substantially involved in influencing antigen binding.

In particular embodiments, choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody. See, e.g., Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies. See, e.g., Carter et al. (1992) Proc. Natl. Acad. Sci. USA, 89:4285; Presta et al. (1993) J. Immunol., 151:2623.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

### 2. Target-Expressing Cells

In some embodiments, the recombinant receptor stimulating agent is a cell that expresses the target recognized by the antigen receptor, i.e. the recombinant receptor stimulating agents is a target-expressing cells. In some embodiments, the target is an antigen of the recombinant receptor and thus, in some cases, the target-expressing cells are antigen-expressing cells. In some embodiments, the recombinant receptor stimulating agent is an antigen-expressing cell, such as a cell expressing a target or an antigen as described above.

In certain embodiments, the cells, e.g., target-expressing cells, such as antigen-expressing cells are exogenous, heterologous, and/or autologous to a subject. In some embodiments, the cells are exogenous to the subject.

In certain embodiments, the target-expressing cells, express a target that is bound by and/or recognized by the recombinant receptor. In some embodiments, the target is an antibody and the target-expressing cells express the antibody. In some embodiments, the target-expressing cells are tumor cells. In particular embodiments, the target-expressing cells are primary cells.

In some embodiments, the target is an antigen recognized by the recombinant receptor and the target-expressing cells are antigen-expressing cells. In certain embodiments, the antigen-expressing cells, express an antigen that is bound by and/or recognized by the recombinant receptor. In some embodiments, the antigen-expressing cells are tumor cells. In particular embodiments, the antigen-expressing cells are primary cells. In some embodiments, the cell line is an immortal cell line. In particular embodiments, the antigen expressing cells are cancerous cells and/or tumor cells. In some embodiments, the antigen-expressing cells are derived from a cancer cell and/or a tumor cells, e.g., human cancer cells and/or human tumor cells. In some embodiments, the antigen-expressing cells are cells from a cancer cell line, optionally a human cancer cell line. In some embodiments, the antigen-expressing cells are cell from a tumor cell line, optionally a human tumor cell line.

In particular embodiments, the antigen-expressing cells are tumor cells. In some embodiments, the antigen-expressing cells are circulating tumor cells, e.g., neoplastic immune cells such as neoplastic B cells (or cells derived from neoplastic B cells).

In particular embodiments, the antigen-expressing cells express anb integrin (avb6 integrin), B cell maturation antigen (BCMA), B7-H3, B7-H6, carbonic anhydrase 9 (CA9, also known as CAIX or G250), a cancer-testis antigen, cancer/testis antigen IB (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD 133, CD13 8, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gplOO), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-AIAI), Human leukocyte antigen A2 HLA-A2), IL-22 receptor alpha (IL-22Ra), IL-13 receptor alpha 2 (IL-13Ra2), kinase insert domain receptor (kdr), kappa light chain, L I cell adhesion molecule (LI-CAM), CE7 epitope of L I-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (KG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRPl, also known as TYRPl or gp75), Tyrosinase related protein 2 (TRP2, also known as dopachrome, tautomerase, dopachrome deltaisomerase or DCT), vascular endothelial growth factor receptor (VEGFR), vascular endothelial growth factor receptor 2 (VEGFR2), Wilms Tumor 1 (WT-1), or a combination thereof. In some embodiments, the antigen-expressing cells express a pathogen-specific or pathogen-expressed antigen, or an antigen associated with a universal tag, and/or biotinylated molecules, and/or molecules expressed by HIV, HCV, HBV or other pathogens. In particular embodiments, the antigen expressing cells express one or more antigens associated with a B cell malignancy, such as any of a number of known B cell markers. In certain embodiments, the antigen-expressing cells express CD20, CD19, CD22, ROR1, CD45, CD21, CD5, CD33, Igkappa, Iglambda, CD79a, CD79b, CD30 or a combination thereof. In some embodiments, the antigen expression-cells express CD19, e.g., human CD19.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In certain embodiments, the antigen-expressing cells are, or are derived from, a tumor cell. In some embodiments, the tumor cell is cancerous. In particular embodiments the tumor cells is non-cancerous. In some embodiments, the tumor cell is or is derived a circulating B cell, such as a circulating B cell capable of forming a tumor in vivo. In some embodiments, the tumor cell is or is derived from a circulating B cell that is a neoplastic, tumorigenic, or cancerous B cell.

In certain embodiments, the tumor cell is, or is derived from, a human cancer cell. In some embodiments, the tumor cell is derived from a cell of a (n) AIDS-related cancer, a breast cancer, a cancer of the digestive/gastrointestinal tract, an anal cancer, an appendix cancer, a bile duct cancer, a colon cancer, a colorectal cancer, an esophageal cancer, a gallbladder cancer, islet cell tumors, pancreatic neuroendocrine tumors, a liver cancer, a pancreatic cancer, a rectal cancer, a small intestine cancer, a stomach (gastric) cancer, an endocrine system cancer, an adrenocortical carcinoma, a parathyroid cancer, a pheochromocytoma, a pituitary tumor, a thyroid cancer, an eye cancer, an intraocular melanoma, a retinoblastoma, a bladder cancer, a kidney (renal cell) cancer, a penile cancer, a prostate cancer, a transitional cell renal pelvis and ureter cancer, a testicular cancer, a urethral cancer, a Wilms' tumor or other childhood kidney tumor, a germ cell cancer, a central nervous system cancer, an extracranial germ cell tumor, an extragonadal germ cell tumor, an ovarian germ cell tumor, a gynecologic cancer, a cervical cancer, an endometrial cancer, a gestational trophoblastic tumor, an ovarian epithelial cancer, a uterine sarcoma, a vaginal cancer, a vulvar cancer, a head and neck cancer, a hypopharyngeal cancer, a laryngeal cancer, a lip and oral cavity cancer, a metastatic squamous neck cancer, a nasopharyngeal cancer, an oropharyngeal cancer, a paranasal sinus and nasal cavity cancer, a pharyngeal cancer, a salivary gland cancer, a throat cancer, a musculoskeletal cancer, a bone cancer, a Ewing's sarcoma, a gastrointestinal stromal tumors (GIST), an osteosarcoma, a malignant fibrous histiocytoma of bone, a rhabdomyosarcoma, a soft tissue sarcoma, a uterine sarcoma, a neurologic cancer, a brain tumor, an astrocytoma, a brain stem glioma, a central nervous system atypical teratoid/rhabdoid tumor, a central nervous system embryonal tumors, a central nervous system germ cell tumor, a craniopharyngioma, an ependymoma, a medulloblastoma, a spinal cord tumor, a supratentorial primitive neuroectodermal tumors and pineoblastoma, a neuroblastoma, a respiratory cancer, a thoracic cancer, a non-small cell a lung cancer, a small cell lung cancer, a malignant mesothelioma, a thymoma, a thymic carcinoma, a skin cancer, a Kaposi's sarcoma, a melanoma, or a Merkel cell carcinoma, or any equivalent human cancer thereof.

In particular embodiments, the tumor cell is derived from a non-hematologic cancer, e.g., a solid tumor. In certain embodiments, the tumor cell is derived from a hematologic cancer. In certain embodiments, the tumor cell is derived from a cancer that is a B cell malignancy or a hematological malignancy. In particular embodiments, the tumor cell is derived from a non-Hodgkin lymphoma (NHL), an acute lymphoblastic leukemia (ALL), a chronic lymphocytic leukemia (CLL), a diffuse large B-cell lymphoma (DLBCL), acute myeloid leukemia (AML), or a myeloma, e.g., a multiple myeloma (MM), or any equivalent human cancer thereof. In some embodiments, the antigen-expressing cell is a neoplastic, cancerous, and/or tumorigenic B cell. Multiple tumor cell lines are known and available and can be selected depending on the antigen recognized by the particular recombinant receptor (e.g. CAR).

Any of a number of tumor cell lines are known and available. Tumor cell lines are known that express particular tumor antigens or surface expression of a tumor antigen can be readily determined or measured by as skilled artisan using any of a variety of techniques, such as by flow cytometry. Exemplary tumor cell lines include, but are not limited to, lymphoma cells (Raji; Daudi; Jeko-1; BJAB; Ramos; NCI-H929; BCBL-1; DOHH-2, SC-1, WSU-NHL, JVM-2, Rec-1, SP-53, RL, Granta 519, NCEP-1, CL-01), leukemia cells (BALL-1, RCH-ACV, SUP-B15); cervical carcinoma cells (33A; CaSki; HeLa), lung carcinoma cells (NCI-H358; A549, H1355, H1975, Calu-1, H1650 and H727), breast cells, (Hs-578T; ZR-75-1; MCF-7; MCF-7/HER2; MCF10A; MDA-MB-231; SKBR-3, BT-474, MDA-MB-231); ovarian cells (ES-2; SKOV-3; OVCAR3; HEY1B); multiple myeloma cells (U266, NCI-H929, RPMI-8226, OPM2, LP-1, L363, MM.1S, MM.1R, MC/CAR, JJN3, KMS11, AMO-1, EJM; MOLP-8). For instance, exemplary CD19-expressing cell lines include, but are not limited to, Raji, Daudi and BJAB; exemplary CD20-expressing cell lines include Daudi, Ramos and Raji; exemplary CD22-expressing cell lines include, but are not limited to, Ramos, Raji, A549, H727, and H1650; exemplary Her2-expressing cell lines include SKOV3, BT-474 and SKBR-3; exemplary BCMA-expressing cell lines include, but are not limited to, RPMI-8226, NCI-H929, MMIS, MMIR and KMS11; exemplary GPRC5D-expressing cell lines include, but are not limited to, AMO-1, EJM, NCI-H929, MM.1S, MM1.R, MOLP-8, and OPM-2; exemplary ROR1-expressing cell lines include, but are not limited to, A549, MDA-MB-231, H1975, BALL-1 and RCH-ACV.

In some embodiments, the target-expressing cell line is a cell line that has been transduced to express the target of the recombinant receptor. In some embodiments, the target is a tumor antigen. In particular embodiments, the antigen-expressing cell line is a cell line that has been transduced to express the tumor antigen. This cell line may be a mammalian cell line, including, but not limited to, human cell lines. In some embodiments, the human cell line may be K562, U937, 721.221, T2, and CIR cells. For instance, the K562 chronic myeloid leukemia cell line may be introduced with a nucleic acid encoding the tumor antigen. In some embodiments, the cell line can be engineered with plasmid vectors or messenger RNAs (mRNAs) that encode the tumor antigen of interest. In some embodiments, the introduction can be by lentivial-based transduction. In some embodiments the cell line (e.g. K562 cells) stably expresses the exogenous nucleic acid encoding the tumor antigen. In some embodiments the exogenous nucleic acid may be integrated into the genome of the cell line (e.g. K562 cell). In some embodiments the exogenous nucleic acid may be integrated into the genome of the cell line (e.g. K562 cell) at a particular locus. In some embodiments, the exogenous nucleic acid may be integrated into the genome of the cell line (e.g. K562 cell) at a genomic safe harbor (GSH). A GSH is a site which supports stable integration and expression of exogenous nucleic acid while minimizing the risk of unwanted interactions with the host cell genome (see e.g. Sadelain et al., Nat Rev Cancer. (2011) 12 (1): 51-8). Several safe GSHs for stable integration of exogenous nucleic acid in human cells have been identified, including AAVS1, a naturally occurring site of integration of AAV virus on chromosome 19; CCR5 gene a chemokine receptor gene also known as an HIV-1 coreceptor; and the human ortholog of the mouse Rosa26 locus (see e.g. Papapetrou and Schambach Mol Ther. (2016) 24 (4): 678-684).

In some embodiments, the target-expressing cells are provided at a fixed amount of the cells of the reporter cells expressing the recombinant receptor (effector cells). In some embodiments, amount is from 100:1 to 0.001 ratio of target-expressing target cells to effector T cells (T:E), such as a titrated amount from 50:1 to 0.050 T:E ratio, from 25:1 to 0.025 T:E ratio, from 12:1 to 0.012:1 T:E ratio, from 10:1 to 0.010 T:E ratio or from 5:1 to 0.5 T:E ratio. In some embodiments, the ratio is or is about from a 12:1 to 0.012:1 T:E ratio. In some embodiments, the ratio is or is about 1:1 to 6:1. The particular ratio can be empirically determined depending on the particular target and the target cells being employed. For instance, the ratio chosen is one that results in a detectable signal in the assay, including a linear dose-response increase in detectable signal across the plurality of titrated amounts of the viral vector used to transduce the reporter T cells.

For instance, the target is an antigen of the recombinant receptor. In some embodiments, the antigen-expressing cell provided at a fixed amount of the cells of the reporter cells expressing the recombinant receptor (effector cells). In some embodiments, amount is from 100:1 to 0.001 ratio of antigen-expressing target cells to effector T cells (T:E), such as a titrated amount from 50:1 to 0.050 T:E ratio, from 25:1 to 0.025 T:E ratio, from 12:1 to 0.012:1 T:E ratio, from 10:1 to 0.010 T:E ratio or from 5:1 to 0.5 T:E ratio. In some embodiments, the ratio is or is about from a 12:1 to 0.012:1 T:E ratio. In some embodiments, the ratio is or is about 1:1 to 6:1. The particular ratio can be empirically determined depending on the particular antigen and the target cells being employed. For instance, the ratio chosen is one that results in a detectable signal in the assay, including a linear dose-response increase in detectable signal across the plurality of titrated amounts of the viral vector used to transduce the reporter T cells.

### C. Measuring Reporter Activity

The methods for assessing potency provided herein include measuring reporter activity of the reporter cell compositions in response to stimulation of recombinant receptors of the cells of the reporter cell composition. As described above, the provided assays allow for measuring activity detectable signal in the reporter cells in response to the incubation with a recombinant receptor stimulating agent, such as described in Section I-A, from a plurality of incubating conditions, where each incubation comprises a different titrated amount of viral vector.

In particular embodiments, the detectable signal is or includes the production and/or secretion of an enzymatic product. In some embodiments, the detectable signal is or includes the production and/or secretion of a bioluminescent factor. In certain embodiments, intensity of light signal is positively correlated with recombinant receptor-dependent activity as a result of luciferase expression.

Suitable techniques for the measurement of the production or secretion of a factor are known in the art. Production and/or secretion of a soluble factor can be measured by determining the concentration or amount of the extracellular amount of the factor, or determining the amount of transcriptional activity of the gene that encodes the factor. Suitable techniques include, but are not limited to assays such as an immunoassay, an aptamer-based assay, a histological or cytological assay, an mRNA expression level assay, an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay, protein microarrays, high-performance liquid chromatography (HPLC), Meso Scale Discovery (MSD) electrochemiluminescence and bead based multiplex immunoassays (MIA). In some embodiments, the suitable technique may employ a detectable binding reagent that specifically binds the soluble factor.

In particular embodiments, the measurement of the soluble factor, e.g., cytokine, is measured by ELISA (enzyme-linked immunosorbent assay). ELISA is a plate-based assay technique designed for detecting and quantifying substances such as peptides, cytokines, antibodies and hormones. In an ELISA, the soluble factor must be immobilized to a solid surface and then complexed with an antibody that is linked to an enzyme. Detection is accomplished by assessing the conjugated enzyme activity via incubation with a substrate to produce a detectable signal. In some embodiments, the recombinant receptor-dependent activity is measured with an ELISA assay.

In certain embodiments, production or secretion, including of light signal, is stimulated in a reporter cell composition that contains recombinant receptor expressing cells, e.g., CAR expressing cells, by a binding molecule capable of binding to the recombinant receptor to stimulate a recombinant receptor-dependent activity, e.g., a CAR-dependent activity. In some embodiments, the binding molecule is an antigen or an epitope thereof that is specific to the recombinant receptor; a cell, e.g., a cell that expresses the antigen; or an antibody or a portion or variant thereof that binds to and/or recognizes the recombinant receptor; or a combination thereof (see e.g., Section I-B above). In certain embodiments, the binding molecule is a recombinant protein that comprises the antigen or epitope thereof that is bound by or recognized by the recombinant receptor.

The duration of the plurality of incubations is contemplated to be commensurate with at least the minimal amount of time for expression of an enzyme (e.g., luciferase) and subsequent detection of product (e.g., luminescence). It is further contemplated that within a type of activity, e.g., enzymatic activity, there may be a difference in time for differing amounts of available substrate. In some embodiments, the plurality of incubations are performed for at or about 15 minutes to at or about 24 hours, such as at or about 2 hours to at or about 6 hours, for example at or about 4 hours. In some embodiments, the plurality of incubations are performed for at or about 30 minutes, 1 hour, 2 hours, 3 hours, 4 hours, 5 hours, 6 hours, 7 hours, 8 hours, 9 hours, 10 hours, 11 hours or 12 hours or any value between any of the foregoing. In some embodiments, the plurality of incubations are performed for at, about, or at least 10, 15, 20, 25, 30, 35, 40, 45, 50, 55, or 60 minutes. In some embodiments, the plurality of incubations are performed for at, about, or at least 30 minutes. In some embodiments, the plurality of incubations are performed for at, about, or at least 60 minutes. In some embodiments, the plurality of incubations are performed for at or about between 10 and 60, 20 and 60, 30 and 60, 40 and 60, 50 and 60 minutes.

In certain embodiments, the detectable signal is a light signal. In some embodiments, cells of the reporter cell composition that contain recombinant receptor expressing cells are incubated in the presence of a binding molecule for an amount of time, and the production and/or secretion of the factor is measured at one or more time points during the incubation. In some embodiments, the cells are incubated with the binding molecule for up to or about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 19 hours, about 20 hours, about 21 hours, about 22 hours, about 23 hours, about 24 hours, about 48 hours, or for a duration of time between 1 hour and 4 hours, between 1 hour and 12 hours, between 12 hours and 24 hours, each inclusive, or for more than 24 hours and the amount of a factor, e.g., a light signal, is detected.

In some embodiments, the binding molecule is a cell that expresses an antigen recognized by the recombinant receptor. In some embodiments, the recombinant receptor is a CAR, and a constant number of the cells of the reporter cell composition are incubated at a plurality of ratios of cells of the reporter cell composition to the cells expressing the antigen including at or about 1:100, 1:75, 1:50, 1:40, 1:30, 1:20, 1:15, 1:14, 1:13, 1:12, 1:11, 1:10, 1:9, 1:8, 1:7, 1:6, 1:5, 1:4, 1:3, 1:2, 1:1, 1:0.5, 1:0.4, 1:0.3, 1:0.2, or 1:0.1, or a range between any of the foregoing, such as at a ratio between 1:1 and 1:10 or 1:0.2 to 1:12, each inclusive. In some embodiments, the plurality of ratios includes any or all of the ratios provided herein.

In some embodiments, the binding molecule is a cell that expresses an antigen recognized by the recombinant receptor. In some embodiments, the recombinant receptor is a CAR, and a number of the cells of the reporter cell composition are incubated with a constant number of cells expressing antigen at a plurality of ratios of cells of the reporter cell composition to the cells expressing the antigen including at or about 1:100, 1:75, 1:50, 1:40, 1:30, 1:20, 1:15, 1:14, 1:13, 1:12, 1:11, 1:10, 1:9, 1:8, 1:7, 1:6, 1:5, 1:4, 1:3, 1:2, 1:1, 1:0.5, 1:0.4, 1:0.3, 1:0.2, or 1:0.1, or a range between any of the foregoing, such as at a ratio between 1:1 and 1:10 or 1:0.2 to 1:12, each inclusive. In some embodiments, the plurality of ratios includes any or all of the ratios provided herein.

In some embodiments, between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, and between about 1×10⁸ and about 1×10¹⁰ cells of the cell composition, each inclusive, are incubated with a constant amount or concentration of binding molecule.

In some embodiments, the cells of the reporter cell composition are incubated with the binding molecule, in a volume of cell media. In certain embodiments, the cells are incubated with the binding molecule in a volume of at least or about 1 μL, at least or about 10 μL, at least or about 25 μL, at least or about 50 μL, at least or about 100 μL, at least or about 500 μL, at least or about 1 mL, at least or about 1.5 mL, at least or about 2 mL, at least or about 2.5 mL, at least or about 5 mL, at least or about 10 mL, at least or about 20 mL, at least or about 25 mL, at least or about 50 mL, at least or about 100 mL, or greater than 100 mL. In certain embodiments, the cells are incubated with the binding molecule in a volume that falls between about 1 μL and about 100 μL, between about 100 μL and about 500 μL, between about 500 μL and about 1 mL, between about 500 μL and about 1 mL, between about 1 mL and about 10 mL, between about 10 mL and about 50 mL, or between about 10 mL and about 100 mL, each inclusive. In certain embodiments, the cells are incubated with the binding molecule in a volume of between about 100 μL and about 1 mL, inclusive. In particular embodiments, the cells are incubated with the binding molecule in a volume of about 500 μL.

In certain embodiments, the measurement of the detectable signal is the amount or concentration, or a relative amount or concentration, of the factor in the reporter cell composition at a time point during or at the end of the incubation for each of the plurality of ratios tested. In particular embodiments, the measurement is subtracted by or normalized to a control measurement. In some embodiments, the control measurement is a measurement from the same cell composition taken prior to the incubation. In particular embodiments the control measurement is a measurement taken from an identical control cell composition that was not incubated with the binding molecule. In certain embodiments, the control is a measurement taken at an identical time point during incubation with the bind molecule from a cell composition that does not contain recombinant receptor positive cells.

In certain embodiments, cells of the reporter cell composition are incubated with the target cells for up to or about 1 hour, about 2 hours, about 3 hours, about 4 hours, about 5 hours, about 6 hours, about 8 hours, about 12 hours, about 18 hours, about 24 hours, about 48 hours, or greater than 48 hours. In some embodiments, a constant number of cells of the therapeutic cell composition are incubated with the cells expressing antigen for about 18 hours, 19 hours, 20 hours, 21 hours, 22 hours, 23 hours, or 24 hours. In some embodiments, a constant number of cells between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, or between about 1×10⁸ and about 1×10¹⁰ cells of the therapeutic cell composition, each inclusive, are incubated with a varying number of the antigen-expressing cells to generate a plurality of ratios. In certain embodiments, a constant amount of cells between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, or between about 1×10⁸ and about 1×10¹⁰ CAR+ cells of the therapeutic cell composition, each inclusive, are incubated with a varying number of antigen-expressing cells to generate a plurality of ratios.

In some embodiments, the measurements of the detectable signal are fit using a mathematical model to produce a dose response curve of the detectable signal. Curve fitting may, in some cases, allow for inference or extrapolation of behavior, e.g., activity of the reporter cells to produce the detectable signal and therefore potency of the viral vector. It is contemplated that any method known in the art to performing curve fitting may be used. In some embodiments, the curve is a sigmoid. In some embodiments, based on the detectable signal measured from each of the plurality of incubations, the titrated ratio that results in a half-maximal detectable signal is determined. In some embodiments, the titrated ratio that results in a half-maximal detectable signal is inferred, extrapolated, or estimated from the dose response curve. In some embodiments, the detectable signal is normalized to the maximum recombinant receptor-dependent activity measured. In some embodiments, the detectable signal is normalized to the upper asymptote of the curve, optionally a range of values of the upper asymptote.

In some embodiments, the methods including assays as described herein may be performed in duplicate or triplicate, or more, to verify the measurements of recombinant receptor-dependent activity. In some cases where the assay is performed, for example, in duplicate, triplicate, or more, the measured recombinant receptor-dependent activity from each of the replicates is used to provide a descriptive statistical measure of the recombinant receptor-dependent activity. For example, in some cases, an average (e.g. arithmetic mean), median, standard deviation, and/or variance of each measure of the recombinant receptor-dependent activity is determined for each of the plurality of ratios test. In some embodiments, an average of each measure of the recombinant receptor-dependent activity is determined. In some embodiments, a standard deviation of each measure of the recombinant receptor-dependent activity is determined. In some embodiments, the average measure of recombinant receptor-dependent activity are fit using a mathematical model to produce or estimate a recombinant receptor-dependent activity curve. In some embodiments, the curve is normalized to the average maximal value. In some embodiments, the curve is normalized to the upper asymptote, optionally an average of a range of values of the upper asymptote. The measures described herein may be used with reference to a reference standard, such as a reference standard described herein, e.g., Section I-D-1.

### D. Determining Viral Vector Potency

The methods provided herein allow for determining a potency of viral vector composition. It is contemplated that the assays described herein may be used to assess the potency of a viral vector composition manufactured by processes such as those described herein (e.g., Section-I), as well as any other manufacturing process that allows for viral vector manufactured to be cultured with reporter cells as described in Section IA1 in the methods provided comprising a plurality of incubations, where each incubation includes culturing different titrated ratios of the viral vector composition (i.e., a vector volume or MOI) with a binding molecule able to stimulate a recombinant receptor-dependent activity in the reporter cell composition. In some embodiments, 1, 2, 3, 4, 5, 6, 7, 8, 9, 10, or more viral vector compositions may be assessed according to the methods provided herein.

By taking measurements of the detectable signal at each of the plurality of ratios tested (i.e., plurality of vector volumes or vector MOIs with a binding molecule), the potency of the viral vector composition may be determined. In some embodiments, the measurements are composites determined by taking an arithmetic mean or median across duplicates, triplicates, or more replicates. In some embodiments, the standard deviation and/or variance of the measurements may be determined. In some embodiments, one or more measurements, including composite measurements, of the recombinant receptor-dependent activity, such as described in Section I-C, of the viral vector composition to the binding molecule, such as a binding molecule described in Section I-B, can be used to determine a potency of a viral vector composition.

In some embodiments, the plurality of incubations at different ratios produces a plurality of measurements to which a curve fitting method may be applied. In some embodiments, the plurality of measurements includes composite measurements (e.g., means or medians). For example, the recombinant receptor-dependent activity measurements can be fit with a curve, e.g., a sigmoid, to allow the inference, extrapolation, or estimation of the behavior (e.g., sensitivity) of the viral vector composition. In some embodiments, a curve fitted to the measurements may be used to estimate behavior (e.g., potency) of the viral vector composition which was not directly examined during the assay. For example, the curve may be used to estimate a lower asymptote; a minimal value; a loss of detection of recombinant receptor-dependent activity; a half-maximal value (e.g., 50% recombinant receptor-dependent activity); a 10%-90%, 20%-80%, 30%-70%, or 40%-60% recombinant receptor-dependent activity range; an upper asymptote; and a maximal value and the ratios at which each of the values or ranges occur.

It is contemplated that any measure, ratio at half-maximal, range, maximal, minimal, asymptote, and composite measures thereof) may be used to determine the potency of a viral vector. In some embodiments, the potency is a relative potency.

### 1. Potency

In some embodiments, the potency of the viral vector composition is defined as the ratio at which one or more or a range of detectable signal measurements occurs. In some embodiments, the one or more or range of measurements are composite measurements, such as a mean or median determined from replicated experiments. In some embodiments, the measurements and ratios are determined from a dose response curve of the measured detectable signal. In some embodiments, the measured detectable signal is normalized to a maximum activity measured for the viral vector composition, e.g., by varying viral vector volume or viral vector MOI. In some embodiments, the dose response curve is normalized to a maximum detectable signal measured for the viral vector composition. In some embodiments, the dose response curve is normalized to an upper asymptote of the recombinant receptor-dependent activity measured for the viral vector composition, optionally an average of measured values across the asymptote.

In some embodiments, the potency of a viral vector composition is the range of ratios over which 10%-90% recombinant receptor-dependent activity occurs, or vice versa. In some embodiments, the range of ratios over which 10%-90% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.1-0.9 or 10%-90%.

In some embodiments, the potency of a viral vector composition is the range of ratios over which 20%-80% recombinant receptor-dependent activity occurs, or vice versa. In some embodiments, the range of ratios over which 20%-80% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.2-0.8 or 20%-80%.

In some embodiments, the potency of a viral vector composition is the range of ratios over which 30%-70% recombinant receptor-dependent activity occurs, or vice versa. In some embodiments, the range of ratios over which 30%-70% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.3-0.7 or 30%-70%.

In some embodiments, the potency of a viral vector composition is the range of ratios over which 40%-60% recombinant receptor-dependent activity occurs, or vice versa. In some embodiments, the range of ratios over which 40%-60% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.4-0.6 or 40%-60%.

In some embodiments, the potency of a viral vector composition is the ratio at which the half-maximal recombinant receptor-dependent activity occurs. In some embodiments, the half-maximal value and ratio at which the half-maximal value occurs is estimated from a recombinant receptor-dependent activity curve. In some embodiments, for example when the recombinant receptor-dependent activity measures or recombinant receptor-dependent activity curve are normalized, the half-maximal recombinant receptor-dependent activity value is 0.5 or 50%.

In some embodiments, for example when the recombinant receptor-dependent activity curve is fit by a sigmoid, a linear portion of the curve is determined. In some embodiments, the potency is a measurement and corresponding ratio from the linear portion of the curve. In some embodiments, the half-maximal value measurement and ratio occur in the linear portion of the curve.

### 2. Relative Potency

The methods provided herein allow for determination of a potency of a viral vector composition relative to a different viral vector composition, e.g., reference standard. This type of potency may be referred to as a relative potency. For example, a viral vector composition assessed according the methods provided herein may be compared to a different viral vector composition (e.g., reference standard, for example as described below), for example assessed according to the methods provided herein to determine how the potencies of the viral vector compositions relate to one another (e.g., titrated as viral vector volume or MOI as described herein). This offers an advantage in that multiple viral vector compositions can be compared to determine which composition has a highest potency.

In some embodiments, the relative potency of the viral vector composition is defined as the ratio(s) (e.g., percentages) at which one or more or a range of recombinant receptor-dependent activity measurements occurs for the viral vector composition compared to the ratio(s) at which one or more or a range of recombinant receptor-dependent activity measurements occurs for the reference standard. In some embodiments, the one or more or range of measurements for one or both the viral vector composition and reference standard are composite measurements, such as a mean or median determined from replicated experiments. In some embodiments, the measurements and ratios for the viral vector composition and the reference standard are determined from a recombinant receptor-dependent activity curve of the measured recombinant receptor-dependent activity for compositions, respectively. In some embodiments, the measured recombinant receptor-dependent activity for the viral vector composition and the reference standard is normalized to a maximum activity measured for the test viral vector composition and reference standard, respectively. In some embodiments, the recombinant receptor-dependent activity curve for the viral vector composition and the reference standard is normalized to a maximum recombinant receptor-dependent activity measured for the viral vector composition and reference standard, respectively. In some embodiments, the recombinant receptor-dependent activity curve for the therapeutic cell composition and the reference standard is normalized to an upper asymptote of the recombinant receptor-dependent activity measured for the viral vector composition and reference standard, respectively, optionally an average of measured values across the asymptote.

In some embodiments, the relative potency of a viral vector composition is the range of ratios over which 10%-90% recombinant receptor-dependent activity occurs, or vice versa, compared to the range over which 10%-90% recombinant receptor-dependent activity occurs, or vice versa, for the standard reference. In some embodiments, the range of ratios over which 10%-90% recombinant receptor-dependent activity occurs for the viral vector composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the viral vector composition and the reference standard are normalized, the range of recombinant receptor-dependent activity value range is from 0.1-0.9 or 10%-90%.

In some embodiments, the relative potency of a viral vector composition is the range of ratios over which 20%-80% recombinant receptor-dependent activity occurs, or vice versa, compared to the range over which 20%-80% recombinant receptor-dependent activity occurs, or vice versa, for the standard reference. In some embodiments, the range of ratios over which 20%-80% recombinant receptor-dependent activity occurs for the therapeutic cell composition and the reference standard is estimated from a recombinant receptor-dependent activity curve for the viral vector composition and the reference standard, respectively. In some embodiments, for example when the recombinant receptor-dependent activity measures or recombinant receptor-dependent activity curve for the viral vector composition and the reference standard are normalized, the range of recombinant receptor-dependent activity value range is from 0.2-0.8 or 20%-80%.

In some embodiments, the relative potency of a viral vector composition is the range of ratios over which 30%-70% recombinant receptor-dependent activity occurs, or vice versa, compared to the range over which 30%-70% recombinant receptor-dependent activity occurs, or vice versa, for the standard reference. In some embodiments, the range of ratios over which 30%-70% recombinant receptor-dependent activity occurs for the viral vector composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the viral vector composition and the reference standard are normalized, the range of recombinant receptor-dependent activity value range is from 0.3-0.7 or 30%-70%.

In some embodiments, the relative potency of a viral vector composition is the range of ratios over which 40%-60% recombinant receptor-dependent activity occurs, or vice versa, compared to the range over which 40%-60% recombinant receptor-dependent activity occurs, or vice versa, for the standard reference. In some embodiments, the range of ratios over which 40%-60% recombinant receptor-dependent activity occurs for the viral vector composition and the reference standard is estimated from a recombinant receptor-dependent activity curve for the viral vector composition and the reference standard, respectively. In some embodiments, for example when the recombinant receptor-dependent activity measures or recombinant receptor-dependent activity curve for the viral vector composition and the reference standard are normalized, the range of recombinant receptor-dependent activity value range is from 0.4-0.6 or 40%-60%.

In some embodiments, the relative potency of a viral vector composition is the ratio at which a specified recombinant receptor-dependent activity (e.g., 10%, 20%, 25%, 30%, 40%, 50%, 60%, 70%, 75%, 80%, or 90% of the maximum) occurs relative to the ratio at which the specified recombinant receptor-dependent activity occurs for the reference standard. In some embodiments, the specified recombinant receptor-dependent activity and ratio at which the specified value occurs for the viral vector composition and the reference standard is determined from a recombinant receptor-dependent activity curve for the therapeutic cell composition and the reference standard, respectively.

In some embodiments, the relative potency of a viral vector composition is the ratio at which the half-maximal recombinant receptor-dependent activity occurs compared to the ratio at which the half-maximal recombinant receptor-dependent activity occurs for the reference standard. In some embodiments, the half-maximal value and ratio at which the half-maximal value occurs for the viral vector composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the viral vector composition and reference standard are normalized, the half-maximal recombinant receptor-dependent activity value is 0.5 or 50%.

In some embodiments, for example when the recombinant receptor-dependent activity curve for viral vector composition and the reference standard are fit by a sigmoid, a linear portion of the curves is determined. In some embodiments, the relative potency is a comparison of the measurement and corresponding ratio from the linear portion of the curve of the viral vector composition and the measurement and corresponding ratio from the linear portion of the curve of the reference standard. In some embodiments, the half-maximal value measurement and ratio for the therapeutic cell composition and reference standard occur in the linear portion of the curve.

In some embodiments, the comparison between the measurements, such as described above, for the viral vector composition and the reference composition is a division. For example, the ratio at which half-maximal recombinant receptor-dependent activity occurs for the therapeutic cell composition is divided by the ratio at which half-maximal recombinant receptor-dependent activity occurs for the reference standard. In some embodiments, the relative potency is expressed as a ratio. In some embodiments, the relative potency is expressed as a percentage.

In some embodiments, for example when the recombinant receptor-dependent activity curve for viral vector composition and the reference standard are fit by a sigmoid and normalized as described above, the relative potency is the difference between the curves. In some embodiments, the difference between the curves is measured for the linear portion of the normalized curves. In some embodiments, normalization of the recombinant receptor-dependent activity curves, e.g., sigmoid curves, for viral vector composition and the reference standard, may be used to directly compare the recombinant receptor-dependent activity curve for viral vector composition and the reference standard.

a. Reference Standard

Particular embodiments contemplate that a measurement of a recombinant receptor-dependent activity (e.g., CAR+ dependent activity) for a viral vector composition can be compared to a reference measurement, (i.e. a reference measure) of a reference standard to, for example, determine a relative potency. In particular embodiments, the reference measurement is a predetermined measurement, or value thereof, of the recombinant receptor-dependent activity of the reference standard. In some embodiments, the recombinant receptor-dependent activity of the reference standard is assessed according to the methods disclosed herein. In some embodiments, the reference standard is a viral vector composition for which titrated ratios resulting in a recombinant receptor-dependent activity have been validated. In some embodiments, the reference standard is a viral vector composition for which titrated ratios resulting in a recombinant receptor-dependent activity have been validated and a curve, e.g., sigmoid, has been fit to the measured activity to generate recombinant receptor-dependent activity curve. In some embodiments, the recombinant receptor-dependent activity curve for the reference standard is normalized. In some embodiments, the recombinant receptor-dependent activity curve is normalized to a maximal measured recombinant receptor-dependent activity. In some embodiments, the recombinant receptor-dependent activity curve is normalized to an upper asymptote of the recombinant receptor-dependent activity curve. In some embodiments, the recombinant receptor-dependent activity curve is normalized to an average value calculated over the upper asymptote of the recombinant receptor-dependent activity curve. In some embodiments, the reference standard is a viral vector composition comprising a validated titrated ratio resulting in a half-maximal recombinant receptor-dependent activity. In some embodiments, the validated titrated ratio resulting in a half-maximal recombinant receptor-dependent activity is determined from the recombinant receptor-dependent activity curve.

In some embodiments, the reference standard is a commercially available viral vector composition. In some embodiments, the reference standard is a viral vector composition manufactured using a manufacturing process that is identical to a manufacturing process used to manufacture the viral vector composition to which it is compared. In some embodiments, the reference standard is a viral vector composition manufactured using a manufacturing process that is different from a manufacturing process used to manufacture the viral vector composition to which it is compared. In some embodiments, the reference standard is from a lot process determined to be representative. In some embodiments, the reference standard is GMP grade. In some embodiments, the reference standard is a viral vector composition comprising an identical recombinant receptor as the therapeutic cell composition to which it is compared. In some embodiments, the reference standard is a viral vector composition comprising a different recombinant receptor as the therapeutic cell composition to which it is compared. In some embodiments, the reference standard is a viral vector composition manufactured from the same subject to which it is compared. In some embodiments, the reference standard is a viral vector composition manufactured from a different subject from which the viral vector composition it which it is compared is manufactured. In some embodiments, the reference standard may be a combination of one or more of those described above.

### II. ARTICLES OF MANUFACTURE AND KITS

Also provided are articles of manufacture, systems, apparatuses, and kits useful in performing the provided methods. Also provided are articles of articles of manufacture, systems, apparatuses, and kits that contain the provided reporter T cells. In some embodiments, the provided articles of manufacture or kits contain reporter T cells for insertion of the nucleic acid sequences encoding candidate binding domains on a test viral vector, e.g., to generate recombinant receptors. In some embodiments, the articles of manufacture or kits can be used in methods of generating a plurality of polynucleotides and/or reporter T cells. In some embodiments, the articles of manufacture or kits provided herein contain T cells, T cell lines and/or a plurality of T cells, such as reporter T cells, described herein.

In some embodiments, the articles of manufacture or kits provided herein contain T cells, T cell lines and/or plurality of T cells, such as any reporter T cells, reporter T cell lines and/or a plurality of reporter T cells described herein. In some embodiments, the T cells, reporter T cell lines and/or a plurality of reporter T cells or any of the modified T cells provided in the articles and/or kits can be used in accordance with used the screening methods described herein. In some embodiments, the articles of manufacture or kits provided herein contain control T cells, reporter T cell lines and/or a plurality of reporter T cells. In some embodiments, the articles of manufacture or kits include one or more reporter T cells, e.g., reporter T cells that contain a reporter molecule, wherein the expression of said reporter molecule is responsive to a signal through the intracellular signaling region. In some embodiments, the articles of manufacture or kits include one or a plurality of reporter T cells, e.g., reporter T cells that contain a reporter molecule and a recombinant receptor, e.g., one of a plurality of recombinant receptors.

In some embodiments, the articles of manufacture or kits include one or more components used to assess the properties of the cells following incubation with a test viral vector, such as cell expressing the recombinant receptors described herein. For example, the articles of manufacture or kits can include binding reagents, e.g., antibodies, antigen-binding fragments thereof, purified or isolated antigen or fragments thereof and/or probes, used to assess particular properties of the introduced candidate recombinant receptors, e.g., cell surface expression of the candidate recombinant receptors, and/or detectable signal produced by the reporter molecule in the reporter T cell, e.g., a Nur77 reporter. In some embodiments, the articles of manufacture or kits can include components that are used for detection of particular properties, such as labeled components, e.g., fluorescently labeled components and/or components that can produce a detectable signal, e.g., substrates that can produce fluorescence or luminescence.

In some embodiments, the articles of manufacture or kits include one or more containers, typically a plurality of containers, packaging material, and a label or package insert on or associated with the container or containers and/or packaging, generally including instructions for use, e.g., instructions for nucleic acid assembly and/or introduction of the assembled nucleic acid molecules or sets of nucleic acid molecules into of cells, such as transfection or transduction of cells used in the provided methods, such as T cells, T cell lines and/or plurality of T cells. In some embodiments, the articles of manufacture and kits include components and/or containers that facilitate high-throughput or large-scale assembly and/or screening. In some embodiments, the articles of manufacture and kits can include high-throughput or large-scale format containers, e.g., multi-well specimen plates, such as a 96-well plate or a 384-well plate.

The articles of manufacture provided herein contain packaging materials. Packaging materials for use in packaging the provided materials are well known to those of skill in the art. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the T cells, T cell lines and/or plurality of T cells are packaged separately. In some embodiments, each container can have a single compartment. In some embodiments, other components of the articles of manufacture or kits are packaged separately, or together in a single compartment.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors include viral vectors, such as retroviral vectors, for example lentiviral or gammaretroviral vectors, having a genome carrying another nucleic acid and capable of inserting into a host genome for propagation thereof.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### IV. EXEMPLARY EMBODIMENTS

Among the provided embodiments are:

1. A method for determining potency of viral vectors, comprising:
a) introducing a titrated amount of a test viral vector encoding a recombinant receptor into a plurality of populations of reporter T cells, wherein each population of reporter T cells is the same and each is introduced with a different amount of the titrated test viral vector, wherein:
each of the reporter T cell populations comprise reporter T cells comprising a nucleic acid sequence encoding a reporter molecule operably linked to a transcriptional regulatory element of a T cell transcription factor;
the recombinant receptor comprises an extracellular binding domain specific to a target, a transmembrane domain and comprises or is complexed with an intracellular signaling region comprising an ITAM-containing domain;
b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule;
c) measuring each of the plurality of populations of reporter T cells for the detectable signal from the reporter molecule; and
d) determining, based on the measured detectable signal, the titrated amount of the test viral vector that results in a half-maximal detectable signal.

2. The method of embodiment 1, wherein the potency is a relative potency and the method further comprises comparing the half-maximal detectable signal of the test viral vector to a half-maximal detectable signal of a reference viral vector standard in the same assay.

3. A method for determining potency of viral vectors, comprising:
a) introducing a titrated amount of a test viral vector encoding a recombinant receptor into a plurality of populations of reporter T cells, wherein each population of reporter T cells is the same and each is introduced with a different amount of the titrated test viral vector, wherein:
each of the reporter T cell populations comprise reporter T cells comprising a nucleic acid sequence encoding a reporter molecule operably linked to a transcriptional regulatory element of a T cell transcription factor;
the recombinant receptor comprises an extracellular binding domain specific to a target, a transmembrane domain and an intracellular signaling region comprising an ITAM-containing domain;
b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule;
c) measuring each of the plurality of populations of reporter T cells for the detectable signal from the reporter molecule; and
d) determining, based on the measured detectable signal, the relative potency of the viral test viral vector by comparing the half-maximal detectable signal to a half-maximal detectable signal of a reference viral vector standard in the same assay.

4. The method of embodiment 2 or embodiment 3, wherein the relative potency is a percentage of the detectable signal of the test viral vector to the reference viral vector standard.

5. The method of embodiment 2 or embodiment 3, wherein the relative potency is a ratio of the detectable signal of the test viral vector to the reference viral vector standard.

6. The method of any of embodiments 1-5, wherein the titrated amount of a test viral vector is a serial dilution of the viral vector.

7. The method embodiment 6, wherein the serial dilution of the viral vector is a serial dilution based on the vector volume.

8. The method of embodiment 6, wherein the serial dilution is a serial dilution based on the viral vector titer.

9. The method of embodiment 8, wherein the viral vector titer is a functional titer, optionally wherein the functional titer is quantified by in vitro plaque assay.

10. The method of embodiment 8, wherein the viral vector titer is a physical titer, optionally wherein the physical titer is quantified via DNA or RNA quantification by a PCR method.

11. The method of embodiment 9 or 10, wherein the viral vector titer is quantified as Infectious Units (IU) per unit of viral vector volume.

12. The method of embodiment 6, wherein the serial dilution is a serial dilution based on the multiplicity of infection (MOI) of the viral vector.

13. The method of embodiment 12, wherein the MOI is quantified via viral vector titer, optionally a functional titer, per number of permissive cells in culture conditions suitable for infection.

14. The method of any of embodiments 1-5, wherein the titrated amount of a test viral vector is a ratio of a constant amount of viral vector to the number of cells in the population of reporter T cells.

15. The method of embodiment 14, wherein the amount of the test viral vector is a volume of the test viral vector.

16. The method of embodiment 14, wherein the amount of the test viral vector is a titer of the test viral vector.

17. The method of embodiment 14, wherein the amount of the test viral vector is a MOI of the test viral vector.

18. The method of any one of embodiments 12, 13, and 17, wherein the MOI is between about 0.001 and 10 particles/cell, optionally at or about 0.01, at or about 0.1, at or about 1.0, or at or about 10 particles/cell or any value between any of the foregoing.

19. The method of embodiments 1-18, wherein the reporter T cell is an immortalized cell line.

20. The method of embodiments 1-5, where in the reporter T cell is a Jurkat cell line or a derivative thereof.

21. The method of embodiment 20, wherein the Jurkat cell line or derivative thereof is Jurkat cell clone E6-1.

22. The method of any of embodiments 1-21, wherein the regulatory element comprises a response element or elements recognized by the transcription factor that is activated upon signaling through the ITAM-containing domain of the recombinant receptor induced by the recombinant receptor stimulating agent.

23. The method of any of embodiments 1-22, wherein the T cell transcription factor is selected from the group consisting of Nur77, NF-κB, NFAT or AP1.

24. The method of any of embodiments 1-23, wherein the T cell transcription factor is Nur77.

25. The method of embodiment 24, wherein the transcriptional regulatory element comprises the Nur77 promoter or portion thereof containing a response element or elements recognized by a transcription factor.

26. The method of embodiment 24 or embodiment 25, wherein the transcriptional regulatory element is a transcriptional regulatory element within an endogenous Nur77 locus in the T cell.

27. The method of any of embodiments 24-26, wherein the nucleic acid sequence encoding the reporter molecule is integrated in the genome of the reporter T cell at or near the endogenous locus encoding Nur77, wherein the reporter molecule is operably linked to a transcriptional regulatory element of the endogenous Nur77 locus.

28. The method of any of embodiments 24-27, wherein the nucleic acid sequence encoding the reporter molecule is integrated by:
a) inducing a genetic disruption at one or more target site(s) at or near the endogenous locus encoding Nur77; and
b) introducing a template polynucleotide comprising a nucleic acid encoding the reporter molecule for knock-in of the reporter molecule in the endogenous locus by homology directed repair (HDR).

29. The method of embodiment 28, wherein the genetic disruption is induced by a CRISPR-Cas9 combination that specifically binds to, recognizes, or hybridizes to the target site.

30. The method of embodiment 29, wherein the RNA-guided nuclease comprises a guide RNA (gRNA) having a targeting domain that is complementary to the target site.

31. The method of any of embodiments 24-30, wherein the nucleic acid encoding the reporter is present within the genome at a site that is at or near the final exon of the endogenous locus encoding Nur77.

32. The method of any of embodiments 28-31, wherein the one or more target site(s) comprise, and/or the nucleic acid is present within the genome at a site comprising, the nucleic acid sequence TCATTGACAAGATCTTCATG (SEQ ID NO:3) and/or GCCTGGGAACACGTGTGCA (SEQ ID NO:4).

33. The method of any of embodiments 1-32, wherein the reporter molecule is or comprises a luciferase, a β-galactosidase, a chloramphenicol acetyltransferase (CAT), a β-glucuronidase (GUS), or a modified form thereof.

34. The method of any of embodiments 1-33, wherein the reporter molecule is a luciferase, optionally firefly luciferase.

35. The method of any of embodiments 1-34, wherein the nucleic acid sequence encoding the reporter molecule further encodes one or more marker(s) that is or comprises a transduction marker and/or a selection marker.

36. The method of embodiment 35, wherein the transduction marker comprises a fluorescent protein, optionally eGFP.

37. The method of any of embodiments 2-36, wherein the reference viral vector standard is a validated viral vector lot that is representative of the same manufacturing process as the test viral vector.

38. The method of embodiment 37, wherein the reference viral vector standard is a viral vector lot produced under good manufacturing practice (GMP).

39. The method of any of embodiments 2-38, wherein the assessment of the reference viral vector standard is carried out in parallel with the test viral vector in the assay.

40. The method of any of embodiments 1-39, wherein the intracellular signaling domain is or comprises an intracellular signaling domain of a CD3 chain, or a signaling portion thereof.

41. The method of any of embodiments 1-40, wherein the intracellular signaling domain is or comprises a CD3-zeta (CD32) chain or a signaling portion thereof.

42. The method of any of embodiments 1-41, wherein the intracellular signaling region further comprises a costimulatory signaling region.

43. The method of embodiment 42, wherein the costimulatory signaling region comprises an intracellular signaling domain of a T cell costimulatory molecule or a signaling portion thereof.

44. The method of embodiment 42 or embodiment 43, wherein the costimulatory signaling region comprises an intracellular signaling domain of a CD28, a 4-1BB or an ICOS or a signaling portion thereof.

45. The method of any of embodiments 1-41, wherein the recombinant receptor is an engineered T cell receptor (eTCR).

46. The method of any of embodiments 1-44, wherein the recombinant receptor is a chimeric antigen receptor (CAR).

47. The method of any of embodiments 1-46, wherein the recombinant receptor stimulating agent is a binding molecule that is or comprises a target antigen or an extracellular domain binding portion thereof, optionally a recombinant antigen, of the recombinant receptor.

48. The method of embodiment 47, wherein the binding molecule is or comprises an extracellular domain binding portion of the antigen and the extracellular domain binding portion comprises an epitope recognized by the recombinant receptor.

49. The method of any of embodiments 1-46, wherein the recombinant receptor stimulating agent is or comprises a binding molecule that is an antibody specific to an extracellular domain of the recombinant receptor.

50. The method of any of embodiments 1-49, wherein the recombinant receptor stimulating agent is immobilized or attached to a solid support.

51. The method of embodiment 50, wherein the solid support is a surface of the vessel, optionally a well of microwell plate, in which the plurality of incubations are performed.

52. The method of embodiment 50, wherein the solid support is a bead.

53. The method of embodiment 52, wherein the beads are from a composition having a concentration of the binding molecule of between or between about 0.5 μg/mL and 500 μg/mL, inclusive, optionally at or about 5 μg/mL, 10 μg/mL, 25 μg/mL, 50 μg/mL, 100 μg/mL or 200 μg/m, or any value between the foregoing.

54. The method of embodiment 52 or embodiment 53, wherein, for the incubating, the beads are added at a ratio of reporter T cells to the beads that is from or from about 5:1 to 1:5, inclusive.

55. The method of any of embodiments 52-54, wherein, for the incubating, the beads are added at a ratio of reporter cells to the beads is from or from about 3:1 to 1:3 or 2:1 to 1:2.

56. The method of any of embodiments 52-55, wherein, for the incubating, the beads are added at a ratio of reporter cells to the beads that is or is about 1:1.

57. The method of any of embodiments 1-46, wherein the recombinant receptor stimulating agent is a target antigen-expressing cell, optionally wherein the cell is a clone, from a cell line, or a primary cell taken from a subject.

58. The method of embodiment 57, wherein the antigen target-expressing cell is a cell line.

59. The method of embodiment 58, wherein the cell line is a tumor cell line.

60. The method of embodiment 57, wherein the antigen target-expressing cell is a cell that has been introduced, optionally by transduction, to express the antigen target of the recombinant receptor.

61. The method of any of embodiments 57-60, wherein, for the incubating, the target antigen-expressing cells are added at a ratio of antigen target-expressing cells to the reporter T cells of from or from about 1:1 to 10:1.

62. The method of any of embodiments 57-61, wherein, for the incubating, the antigen target-expressing cells are added at a ratio of antigen target-expressing cells to the reporter T cells of from or from about 1:1 to 6:1.

63. The method of any of embodiments 1-62, wherein the plurality of incubations are performed in a flask, a tube, or a multi-well plate.

64. The method of any of embodiments 1-63, wherein the plurality of incubations are each performed individually in a well of a multi-well plate.

65. The method of embodiment 63 or embodiment 64, wherein the multi-well plate is a 96-well plate, a 48-well plate, a 12-well plate or a 6-well plate.

66. The method of any of embodiments 1-65, wherein the detectable signal is measured using a plate reader.

67. The method of embodiment 66, wherein the detectable signal is luciferase and the plate reader is a luminometer plate reader.

68. The method of any of embodiments 1-67, wherein the virial vector is an adenoviral vector, adeno-associated viral vector, or a retroviral vector 69. The method of any of embodiments 1-68, wherein the viral vector is a retroviral vector.

70. The method of any of embodiments 1-69, wherein the viral vector is a lentiviral vector.

71. The method of embodiment 70, wherein the lentiviral vector is derived from HIV-1. 72. The method of any of embodiments 1-71, wherein the detectable signal is luciferase luminescence.

### V. EXAMPLES

The following examples are included for illustrative purposes only and are not intended to limit the scope of the invention.

### Example 1 Generation of Nur77-Luciferase-EGFP Reporter Cell Line

An exemplary reporter cell line was generated containing a Nur77-Luciferase-EGFP knock-in reporter. Orphan nuclear hormone receptor Nur77 (also called Nr4a1; exemplary human Nur77 DNA sequence set forth in SEQ ID NO:1, encoding the polypeptide set forth in SEQ ID NO:2) is an immediate-early response gene induced by activation of signal from the T cell receptor and/or via molecules containing immunoreceptor tyrosine-based activation motif (ITAM). A Jurkat T cell clone E6-1 (ATCC® TIB-152™) was engineered by co-transfection of a vector encoding a Nur77-targeting guide RNA (gRNA)/CRISPR-Cas9 (gRNA targeting domain sequences set forth in SEQ ID NOS: 3 and 4), and exemplary template DNA for knock-in of the reporter by homology directed repair (HDR; template DNA sequence set forth in SEQ ID NO: 5). The template DNA contained polynucleotides encoding two T2A ribosomal skip elements (sequence set forth in SEQ ID NO:6, encoding polypeptide sequence set forth in SEQ ID NO: 7) on either side of Firefly Luciferase 2 (FFLuc2) (sequence set forth in SEQ ID NO:8; encoding polypeptide sequence set forth in SEQ ID NO:9), as well as the monomeric Enhanced Green Fluorescent Protein (EGFP) at the 5′ end (sequence set forth in SEQ ID NO:10, encoding polypeptide sequence set forth in SEQ ID NO: 11). These regions were flanked on either side of the coding sequences by the 5′ homology arm (set forth in SEQ ID NO: 12, containing 2 silent mutations to reduce cleavage of the template DNA by CRISPR/Cas9) and the 3′ homology arm (set forth in SEQ ID NO: 13), homologous to sequences surrounding the stop codon of the endogenous Nur77 gene. The T2A-FFLuc2-T2A-EGFP encoding sequences were targeted to be inserted in-frame with the endogenous Nur77 gene, prior to the stop codon.

Cells were transfected and incubated with phorbol 12-myristate 13-acetate (PMA) and ionomycin for 18 hours and assessed for EGFP expression. Cells that expressed EGFP were sorted using flow cytometry. Knock-in at the Nur77 locus was confirmed by DNA sequencing.

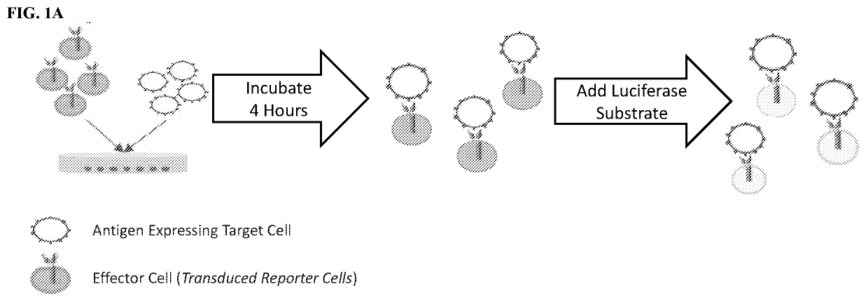

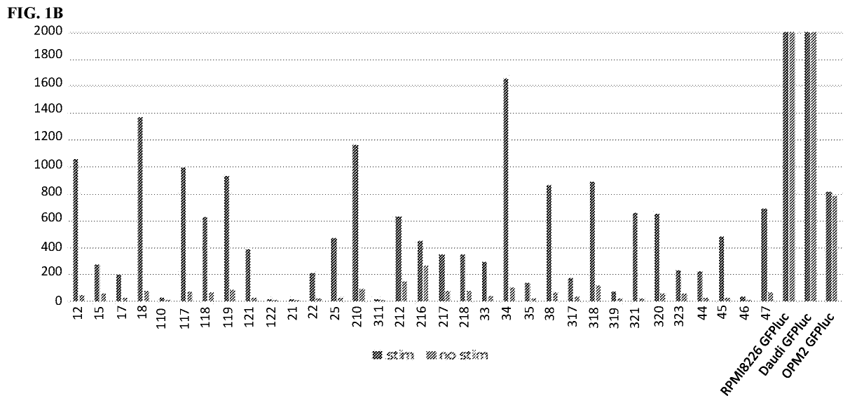

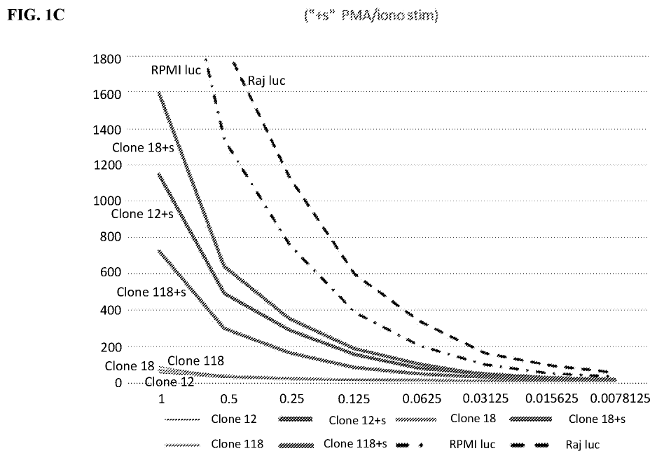

Sorted EGFP+ cells were then incubated with ONE-GLOW Luciferase Assay Buffer and Substrate (Promega), a specific substrate for the luciferase enzyme, following stimulation of the cells with PMA-ionomycin. Following incubation with substrate at room temperature for at least three minutes to allow for complete cell lysis, luciferase activity was measured with a plate luminometer in relative luminescence units (RLU). Previously established cell lines expressing luciferase were used as positive controls, and unmodified parental cells were used as negative controls. An exemplary diagram is shown in FIG. 1A.

As shown in FIG. 1B, many EGFP+ tested clones showed luciferase enzymatic activity in the presence of activation agonists and substrate. Of these, three exemplary EGFP+/Luc+ cell lines were assessed quantitatively in response to PMA/ionomycin stimulation. Cells were incubated with serial dilution of PMA/ionomycin prior to the addition of the ONE-GLOW Luciferase Assay Substrate as previously described. One Burkitt's lymphoma (Raji) and one multiple myeloma (RPMI 8226) cell line with constitutively active Luciferase enzymes were chosen as controls. As shown in FIG. 1C, a dose-dependent decrease in luciferase activity was observed with decreasing PMA/ionomycin concentration. The results are consistent with the utility of the Nur77-FFLuc2-EGFP reporter construct in assessing dose-dependent stimulation of the reporter cells using PMA/ionomycin.

### Example 2 Assessment of Viral Vector Potency Using the Nur77-Luciferase-EGFP Reporter Cell Line Via Vector Volume

The specific ability or capacity of a product, such as a lentiviral vector, to achieve a defined biological effect is its biological activity, and potency is the quantitative measure of that biological activity. Potency is therefore based on the attribute of the vector which is linked to the relevant biological properties, including efficiency of transduction of target cells. To assess viral vector potency, transduction efficiency of a lentiviral vector encoding a chimeric antigen receptor (CAR) was measured using the stably transfected Nur77-FFLuc2-EGFP Jurkat cell reporter line generated as described in Example 1.

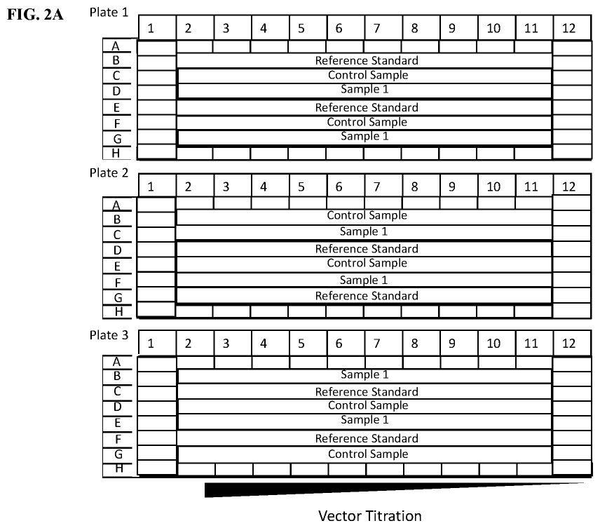

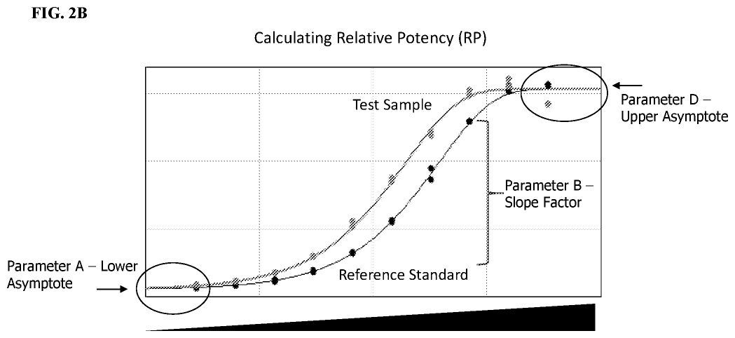

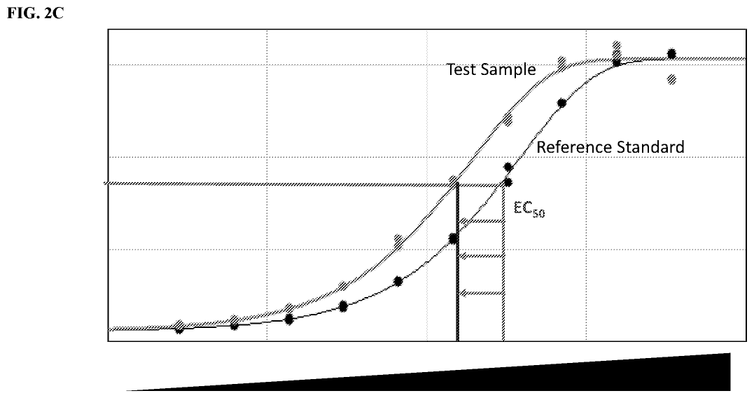

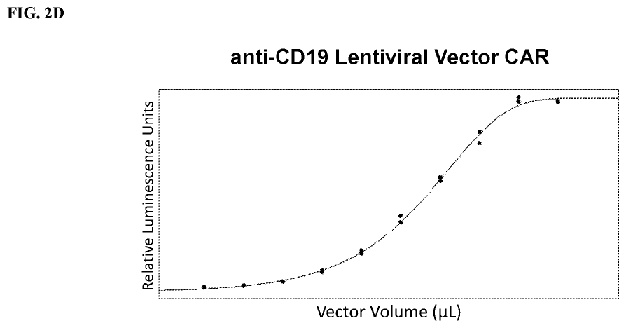

The vector potency assay utilized a 3-plate assay format in which the position of each sample was rotated among the plates to reduce sources of bias due to placement of samples. The vector was titrated from left to right to generate a 10-point dose response curve. An exemplary plate assay set up is shown in FIG. 2A.

The Jurkat reporter cell line was transduced with serially diluted lentiviral vector containing nucleic acid encoding an exemplary CAR. The titrated amounts of serially diluted retroviral vector was added individually in duplicate to wells of a multi-well plate that had been plated with the Jurkat reporter cells. The exemplary CAR included an antigen-binding domain directed against a target antigen (e.g. CD19), a transmembrane domain, and an intracellular signaling region containing a CD3-zeta derived intracellular signaling region and a costimulatory signaling domain. The cells were incubated under conditions sufficient for integration of the CAR construct into the genome of cell. A reference standard was also included that was a lentiviral vector containing the same nucleic acid encoding the CAR as the test lentiviral vector and that was produced from a lot process that was determined to be representative. In some cases, the reference standard can be a lot that has been previously validated for good manufacturing practice (GMP), such as described in Example 4. In such examples, a further control lentiviral vector can also be included for comparison in which the control is a lentiviral vector from a representative lot process, but that has not yet been validated for GMP.

Following the transduction, the CAR-transduced Nur77-FFLuc2-EGFP Jurkat cell reporter cells were then co-cultured with target cells expressing the antigen recognized by the CAR, in this example Raji cells, which are an immortalized Burkitt's lymphoma cell line that endogenously expresses surface CD19. The antigen-expressing target cells were added to the wells of the micro-well plate at a target to effector ratio (T:E) of between 1:1-6:1. Following co-culture at temperatures in media conducive for cell maintenance, luciferase specific substrate was added and the relative luminescence was measured on a plate reader as previously described.

The percent (%) relative potency for the viral vector was determined by using a constrained 5-parameter logistic curve. FIG. 2B depicts an exemplary dose response curve for an exemplary test sample, in which the vector volume (in microliters) is plotted on the x-axis and the relative luciferase units (RLU) on the y-axis, which is directly proportional to vector function. The dose response curve of the exemplary test sample demonstrated the reference standard and test sample have suitable biological equivalence in the assay, and pass other system suitability criteria. This includes criteria for coefficient of variation (CV), R2, and equivalency of the upper asymptote, slope factor, and lower asymptote, shown in FIG. 2B. The upper asymptote (Parameter D) was determined as the mean of duplicate responses at the maximum dose with a difference in the maximum effect between upper asymptotes in a test condition. Similarly, the lower asymptote (Parameter A) was determined as the mean of duplicate responses at the minimal dose with a difference in the minimal effect between lower asymptotes in a test condition.

After biological equivalence has been established the reference standard and test sample dose response curves were constrained. The ratio of the test sample's 50% effective concentration (EC₅₀) compared to the reference standard's EC₅₀ was calculated for each. The results were averaged and reported as Mean % Relative Potency compared to the reference standard; see FIG. 2C. The curve shift to the left indicates an increase in potency of the test sample compared to the reference standard (whereas a curve shift to the right would indicate a decrease in potency compared to the reference standard).

A similar dose response curve is depicted in FIG. 2D for a further exemplary test lot of lentiviral vector encoding an anti-CD19 CAR. The dose response curve can be used to measure the titrated amount that results in a half-maximal detectable signal as a measure of viral vector potency. In some aspects, the relative potency of the viral test viral vector can be determined by comparing the half-maximal detectable signal to a half-maximal detectable signal of a reference viral vector standard in the same assay, using methods as described above.

These results show that the Nur77-FFLuc2-EGFP Jurkat T cell reporter line can be used to assess and compare potencies of viral vectors.

### Example 3 Assessment of Viral Vector Potency Using the Nur77-Luciferase-EGFP Reporter Cell Line Via Multiplicity of Infection (MOI)

To assess viral vector potency, transduction efficiency of a lentiviral vector encoding a chimeric antigen receptor (CAR) was measured using the stably transfected Nur77-FFLuc2-EGFP Jurkat cell reporter line generated as described in Example 1.

This vector potency assay utilized an assay format in which the vector was titrated to generate a range of MOI (IU/cell).

The Jurkat reporter cell line was transduced with the titrated amount of lentiviral vector encoding the CAR. The titrated amount of lentiviral vector was added individually in duplicate to wells of a multi-well plate that had been plated with the Jurkat reporter cells. The exemplary CAR included an antigen-binding domain directed against a target antigen (e.g. BCMA), a transmembrane domain, and an intracellular signaling region containing a CD3-zeta derived intracellular signaling region and a costimulatory signaling domain. The cells were incubated under conditions sufficient for integration of the CAR construct into the genome of cell.

Following the transduction, the CAR-transduced Nur77-FFLuc2-EGFP Jurkat cell reporter cells were then co-cultured with BCMA-expressing target cells. Following co-culture at temperatures in media conducive for cell maintenance, luciferase specific substrate was added and the relative luminescence was measured on a plate reader as previously described.

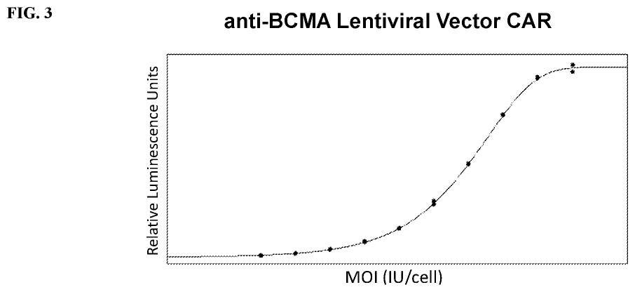

FIG. 3 depicts an exemplary dose response curve for an exemplary test sample, in which the vector MOI (in IU/cell) is plotted on the x-axis and the relative luciferase units (RLU) on the y-axis. The dose response curve can be used to measure the titrated amount that results in a half-maximal detectable signal as a measure of viral vector potency. In some aspects, the relative potency of the viral test viral vector can be determined by comparing the half-maximal detectable signal to a half-maximal detectable signal of a reference viral vector standard in the same assay, using methods as described in Example 2.

These data support that the Nur77-FFLuc2-EGFP Jurkat T cell reporter line can be used to assess and compare potencies of viral vectors.

### Example 4 Method Qualification of Exemplary Vector Potency Assay

To assess the vector potency assay for qualifications according to standard Good Manufacturing Practice (GMP) principles, experiments were carried out to determine the accuracy, precision, repeatability, linearity and specificity of the assay conducted using vector volume as described in Example 2.

Briefly, Nur77-FFLuc2-EGFP Jurkat T cell reporter cells were transfected with test, control and reference vector lots encoding the same exemplary CAR, substantially as described in Example 2. Then, the following qualification parameters were evaluated: accuracy, precision (including repeatability and intermediate precision), linearity, range, and specificity (including antigen-specificity, stability-indicating specificity, and representative material).

### A) Accuracy and Precision

To assess accuracy, precision and repeatability of a reference vector lot from a characterized process determined to be representative was assayed by multiple operators at several levels of percent relative potency. For example, for assessing 200% relative potency a 2× the volume of the 100% reference standard control was used to transduce the Jurkat reporter cell line, and for assessing 50% relative potency a half the volume of the reference standard control was used. A range from 50-200% relative potency was tested (e.g. 50%, 71%, 100%, 141% and 200%)

For assays conducted to measure either of relative accuracy and intermediate precision, at least 3 operators conducted separate experiments over multiple test days to assess the percent recovery. For assays to measure repeatability, a single operator performed 3 experiments with the same test conditions. Using the vector potency assay described in Example 2, relative accuracy target of 80-120% was met and intermediate precision and repeatability targets of ≤20% CV were met, as shown in Tables E1 and E2 below.

TABLE E1
Accuracy and Precision

| Levels (% Relative Potency) | 200% | 141% | 100% | 71% | 50% | Overall |
|---|---|---|---|---|---|---|
| Relative Accuracy | 101 | 99 | 99 | 101 | 100 | 100 |
| (% Recovery) | | | | | | |
| 90% CI of Mean | 97-104 | 90-109 | 93-104 | 89-113 | 98-103 | 98-102 |
| (% Recovery) | | | | | | |
| IP (% CV) | 4 | 6 | 7 | 7 | 3 | 5 |
| 90% CI of IP | 5-18 | 5-35 | 4-14 | 3-22 | 1-4 | 4-7 |

TABLE E2
Repeatability

| | Relative Potency at 100% |
|---|---|
| Relative Accuracy (% Recovery) | 102 |
| Repeatability (% CV) | 2 |
| 90% CI of Repeatability | 1-4% |

### B) Linearity

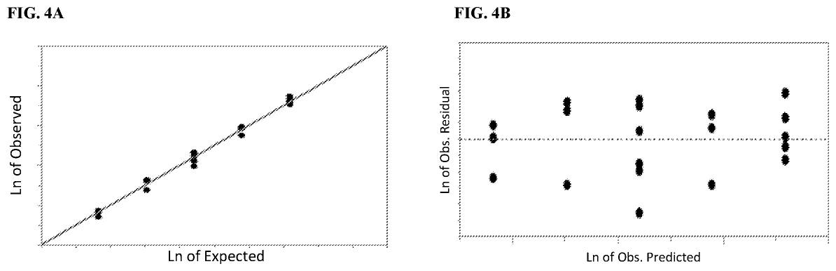

In order to ensure the method demonstrated linearity, the line of best fit was calculated using the accuracy and intermediate precision as described above and is depicted in FIG. 4A. Briefly, the use of linear assays in GMP method qualification is a significant challenge, as existing methods are often limited to parallelism (i.e., in contrast to true dose response curves) as a result of inaccuracies of calculations at the upper asymptotes. The summary of fit can be seen below in Table E3. As shown, conformance to acceptance criteria for accuracy and intermediate precision in at least five consecutive levels the method demonstrated linearity. A linear distribution with a slope of about 1 was observed. Additionally, residual distribution was not bias to one side or the other as shown in FIG. 4B. These data indicates that the residuals (and hence the error) are also normally distributed.

TABLE E3
Summary of Fit

| | |
|---|---|
| R2 | 0.992 |
| Slope (90% CI) | 0.999 (0.966, 1.032) |
| Y-intercept (90% CI) | 0.003 (−0.151, 0.157) |

Taken together, these data support that the assay displays conformance to all acceptance criteria for linearity. Further, these data support that this assay has a linear range from at least 50% to 200%.

### C) Specificity

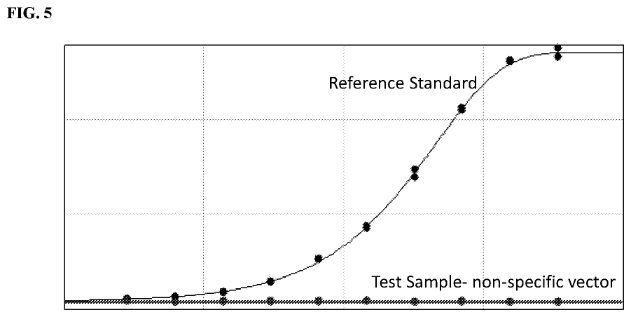

Antigen-specificity was demonstrated as the non-specific vector failed assay acceptance criteria. Briefly, a non-specific vector was used to assess antigen specificity of the reporter cell assay (i.e., specific T cell transduction). A non-specific vector was chosen that would not interact with target cells, specifically a vector that should not be stimulated by the presence of the specific antigen on the target cells. As shown in FIG. 5, the non-specific vector failed to produce any measured output (Y axis) at any volume (X axis), demonstrating antigen specificity of the assay.

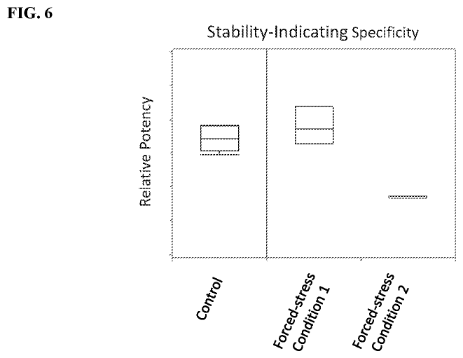

Specificity of the assay was also assessed as stability indicating. Briefly, 3 separate vials of identical vector were thawed for a first forced degradation event (i.e., forced stress). One vial was immediately re-frozen as a control, while the other two underwent two separate temperate stress protocols. As shown in FIG. 6, one protocol resulted in stable vector relatively comparable to the single forced degradation control, while the other forced stress condition resulted in a decrease in relative potency of the vector. These data support that the assay is stability indicating, and that there are degrading conditions which the assay can detect.

### D) Conclusions

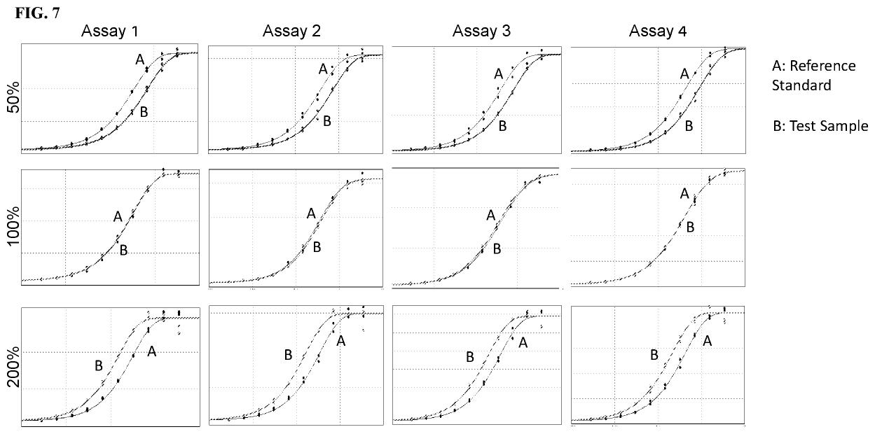

These data support the use of the exemplary assay as a qualified method of determining vector potency. Specifically the data show the method is highly accurate, precise, linear across a wide range (at least 50%-200%), antigen-specific, and stability-indicating. Exemplary readouts across 4 independent assays performed by separate operators are shown in FIG. 7

Further, the assay format reduces commonly observed bioassay biases and addresses many of the challenges cell and gene therapy potency assays face during development, including biological equivalence. Some of these common biases that may be reduced by this assay format include: plate location bias, operator and day to day variability, cell passage age, etc. This allows for results to be compared across operators, across assays, across study days, and across vector lots. Finally, system suitability and assay acceptance criteria are ideal for method trending, which is required for ensuring the assay remains in its validated state through method trending. This allows for consistent monitoring across the assays performance over time at a test site to ensure standards are met and the process remains in the state of control.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Table of Sequences

| SEQ | | |
|---|---|---|
| ID | Sequence | Description |
| 1 | ttcctggtgt aagctttggt atggatggtg gccgtctccc tacagactgg | Human Nur77 DNA |
| | gagctgttagagggcaggga tcctagctga cacatctatg tcctcgcctt ggttggaggc | NCBI Reference |
| | ctccaccatggacagaggcc aggccctgcc cctcccaggc agcctggctc cttctgctgg | Sequence: |
| | gccctgaagg cagacgggat aatgtggttg gccaaggcct gttggtccat ccagagtgag | NM_001202233.1 |
| | atgccctgta tccaagccca atatgggaca ccagcaccga gtccgggacc ccgtgaccac | |
| | tggcaagcg accccctgac ccctgagttc atcaagccca ccatggacct ggccagcccc | |
| | gaggcagccc ccgctgcccc cactgccctg cccagcttca gcaccttcat ggacggctac | |
| | acaggagagt ttgacacctt cctctaccag ctgccaggaa cagtccagcc atgctcctca | |
| | gcctcctcct cggcctcctc cacatcctcg tcctcagcca cctcccctgc ctctgcctcc | |
| | ttcaagttcg aggacttcca ggtgtacggc tgctaccccg gccccctgag cggcccagtg | |
| | gatgaggccc tgtcctccag tggctctgac tactatggca gcccctgctc ggccccgtcg | |
| | ccctccacgc ccagcttcca gccgccccag ctctctccct gggatggctc cttcggccac | |
| | ttctcgccca gccagactta cgaaggcctg cgggcatgga cagagcagct gcccaaagcc | |
| | tctgggcccc cacagcctcc agccttcttt tccttcagtc ctcccaccgg ccccagcccc | |
| | agcctggccc agagccccct gaagttgttc ccctcacagg ccacccacca gctgggggag | |
| | ggagagagct attccatgcc tacggccttc ccaggtttgg cacccacttc tccacacctt | |
| | gagggctcgg ggatactgga tacacccgtg acctcaacca aggcccggag | |
| | cggggcccca ggtggaagtg aaggccgctg gctgtgtgt ggggacaacg cttcatgcca | |
| | gcattatggt gtccgcacat gtgagggctg caagggcttc ttcaagcgca cagtgcagaa | |
| | aaacgccaag tacatctgcc tggctaacaa ggactgccct gtggacaaga ggggcgaaa | |
| | ccgctgccag ttctgccgct tccagaagtg cctggcggtg ggcatggtga aggaagttgt | |
| | ccgaacagac agcctgaagg ggcgggggg cggctacct tcaaaaccca | |
| | agcagccccc agatgcctcc cctgccaatc tcctcacttc cctggtccgt gcacacctgg | |
| | actcagggcc cagcactgcc aaactggact actccaagtt ccaggagctg gtgctgcccc | |
| | actttgggaa ggaagatgct ggggatgtac agcagttcta cgacctgctc tccggttctc | |
| | tggaggtcat ccgcaagtgg gcggagaaga tccctggctt tgctgagctg tcaccggctg | |
| | accaggacct gttgctggag tcggccttcc tggagctctt catcctccgc ctggcgtaca | |
| | ggtctaagcc aggcgagggc aagctcatct tctgctcagg cctggtgcta caccggctgc | |
| | agtgtgcccg tggcttcggg gactggattg acagtatcct ggccttctca aggtccctgc | |
| | acagcttgct tgtcgatgtc cctgccttcg cctgcctctc tgcccttgtc ctcatcaccg | |
| | accggcatgg gctgcaggag ccgcggcggg tggaggagct gagaaccgc | |
| | tcgccagct gcctgaagga gcacgtggca gctgtggcgg gcgagcccca cagccagc | |
| | tgcctgtcac gtctgttggg caaactgccc gagctgcgga ccctgtgcac ccagggcctg | |
| | cagcgcatct tctacctcaa gctggaggac ttggtgcccc ctccacccat cattgacaag | |
| | atcttcatgg acacgctgcc cttctgaccc ctgcctggga acacgtgtgc acatgcgcac | |
| | tctcatatgc caccccatgt gcctttagtc cacggacccc cagagcaccc ccaagcctgg | |
| | gcttgagctg cagaatgact ccaccttctc acctgctcca ggaggtttgc agggagctca | |
| | agcccttggg gagggggatg ccttcatggg ggtgacccca cgatttgtct tatccccccc | |
| | agcctggccc cggcctttat gttttttgta agataaaccg tttttaacac atagcgccgt | |
| | gctgtaaata agcccagtgc tgctgtaaat acaggaagaa agagcttgag gtgggagcgg | |
| | ggctgggagg aagggatggg ccccgccttc ctgggcagcc tttccagcct cctgctggct | |
| | ctctcttcct accctccttc cacatgtaca taaactgtca ctctaggaag aagacaaatg | |
| | acagattctg acatttatat ttgtgtattt tcctggattt atagtatgtg acttttctga | |
| | ttaatatatt | |
| | taatatattg aaaaaaaat agacatgtag ttggaactga aaaaaaaaaa aaa | |
| 2 | MWLAKACWSI QSEMPCIQAQ YGTPAPSPGP RDHLASDPLT | Human Nur77 |
| | PEFIKPTMDL ASPEAAPAAP TALPSFSTFM DGYTGEFDTF | NCBI Reference |
| | LYQLPGTVQP CSSASSSASS TSSSSATSPA SASFKFEDFQ | Sequence: |
| | VYGCYPGPLS GPVDEALSSS GSDYYGSPCS APSPSTPSFQ | NP_001189162.1 |
| | PPQLSPWDGS FGHFSPSQTY EGLRAWTEQL PKASGPPQPP | |
| | AFFSFSPPTG PSPSLAQSPL KLFPSQATHQ LGEGESYSMP | |
| | TAFPGLAPTS PHLEGSGILD TPVTSTKARS GAPGGSEGRC | |
| | AVCGDNASCQ HYGVRTCEGC KGFFKRTVQK | |
| | NAKYICLANK DCPVDKRRRN RCQFCRFQKC | |
| | LAVGMVKEVV RTDSLKGRRG RLPSKPKQPP DASPANLLTS | |
| | VRAHLDSGP STAKLDYSKF QELVLPHFGK EDAGDVQQFY | |
| | DLLSGSLEVI RKWAEKIPGF AELSPADQDL LLESAFLELF | |
| | ILRLAYRSKP GEGKLIFCSG LVLHRLQCAR GFGDWIDSIL | |
| | AFSRSLHSLL VDVPAFACLS ALVLITDRHG LQEPRRVEEL | |
| | QNRIASCLKE HVAAVAGEPQ PASCLSRLLG KLPELRTLCT | |
| | QGLQRIFYLK LEDLVPPPPI IDKIFMDTLP | |
| 3 | CAUGAAGAUCUUGUCAAUGA | Human Nur77 |
| | | gRNA 1 |
| | | targeting |
| | | domain |
| 4 | UGCACACGUGUUCCCAGGC | Human Nur77 |
| | | gRNA 2 |
| | | targeting |
| | | domain |
| 5 | cacctaaatt gtaagcgtta atattttgtt aaaattcgcg ttaaattttt gttaaatcag | Nur77 Knock-in |
| | ctcatttttt aaccaatagg ccgaaatcgg caaaatccct tataaatcaa aagaatagac | construct |
| | cgagataggg ttgagtgttg ttccagtttg gaacaagagt ccactattaa agaacgtgga | sequence |
| | ctccaacgtc aaagggcgaa aaaccgtcta tcagggcgat ggcccactac gtgaaccatc | |
| | accctaatca agttttttgg ggtcgaggtg ccgtaaagca ctaaatcgga accctaaagg | |
| | gagcccccga tttagagctt gacggggaaa gccggcgaac gtggcgagaa | |
| | aggaagggaa gaaagcgaaa ggagcgggcg ctagggcgct ggcaagtgta | |
| | gcggtcacgc tgcgcgtaac caccacaccc gccgcgctta atgcgccgct | |
| | acagggcgcg tcccattcgc cattcaggct gcgcaactgt tgggaagggc gatcggtgcg | |
| | ggcctcttcg ctattacgcc agctggcgaa | |
| | agggggatgt gctgcaaggc gattaagttg ggtaacgcca gggttttccc agtcacgacg | |
| | ttgtaaaacg acggccagtg agcgcgcgta atacgactca ctatagggcg aattgggtac | |
| | caatctcact atgttgcccg agctggtctc gaactcctgg gctcaaatga tcctcctgtc | |
| | tcagcctcct aaagtgctgg gattacaggt gtgagccacc acgcctagcc cttcactgtg | |
| | acttctgaca gtgcagatca gattggttgt gcctgttttg gactttatgt aaatgtagtt | |
| | ctgcaggatg gaatctggtg ttgaatgcag aggttttcag atttctctgt tttttaaagg | |
| | aaagaatcca ccctcgttca ttttttcact taaattgcac aggggaccca acgatataga | |
| | acacaatcag aggtactctg ggctgaggga gtgctgagtt ctgaggctgg gtttctcaga | |
| | acagtctaga ttttaaaaac ccaatgatct agccagaaaa cgtaggttag gattttattt | |
| | cccgtttgtg accctgggca agtcattagc ctcctgggcc tcgggttctc acttggagta | |
| | tgaggataat gagggttact gcttctcaga cttgtgacga tgcttactaa tggccaacat | |
| | gtgaatgcgc ttttgtgaag tgccagcaga gcatgagggg tggtcagggg cagcagtttt | |
| | aggggcctgg gggaggctgg ggctttgggg gcctggttct cagatgtaca gctaatcctg | |
| | tacccttccc gcagaccggc atgggctgca ggagccgcgg cgggtggagg | |
| | agctgcagaa | |
| | ccgcatcgcc agctgcctga aggagcacgt ggcagctgtg gcgggcgagc | |
| | cccagccagc cagctgcctg tcacgtctgt tgggcaaact gcccgagctg cggaccctgt | |
| | gcacccaggg cctgcagcgc atcttctacc tcaagctgga ggacttggtg ccccctccac | |
| | ctatcatcga caagatcttc atggacacgc tgcccttcgg atccggagaa ggacggggct | |
| | ctctgcttac atgtggcgac gttgaggaaa accccggacc tatggaagat gccaaaaaca | |
| | ttaagaaggg | |
| | cccagcgcca ttctacccac tcgaagacgg gaccgccggc gagcagctgc | |
| | acaaagccat gaagcgctac gccctggtgc ccggcaccat cgcctttacc gacgcacata | |
| | tcgaggtgga cattacctac gccgagtact tcgagatgag cgttcggctg gcagaagcta | |
| | tgaagcgcta tgggctgaat acaaaccatc ggatcgtggt gtgcagcgag aatagcttgc | |
| | agttcttcat gcccgtgttg ggtgccctgt tcatcggtgt ggctgtggcc ccagctaacg | |
| | acatctacaa cgagcgcgag ctgctgaaca gcatgggcat cagccagccc accgtcgtat | |
| | tcgtgagcaa | |
| | gaaagggctg caaaagatcc tcaacgtgca aaagaagcta ccgatcatac aaaagatcat | |
| | catcatggat agcaagaccg actaccaggg cttccaaagc atgtacacct tcgtgacttc | |
| | ccatttgcca cccggcttca acgagtacga cttcgtgccc gagagcttcg accgggacaa | |
| | aaccatcgcc ctgatcatga acagtagtgg cagtaccgga ttgcccaagg gcgtagccct | |
| | accgcaccgc accgcttgtg tccgattcag tcatgcccgc gaccccatct tcggcaacca | |
| | gatcatcccc gacaccgcta tcctcagcgt ggtgccattt caccacggct tcggcatgtt | |
| | caccacgctg ggctacttga tctgcggctt tcgggtcgtg ctcatgtacc gcttcgagga | |
| | ggagctattc ttgcgcagct tgcaagacta taagattcaa tctgccctgc tggtgcccac | |
| | actatttagc ttcttcgcta agagcactct catcgacaaa tacgacctaa gcaacttgca | |
| | cgagatcgcc agcggcgggg cgccgctcag caaggaagtc ggcgaggccg | |
| | tggccaaacg cttccaccta cccggcatcc gccagggcta cggcctgaca gaaacaacca | |
| | gcgccattct gatcaccccc gaaggggacg acaagcctgg cgcagtaggc | |
| | aaggtggtgc ccttcttcga ggctaaggtg gtggacttgg acaccggcaa gacactgggt | |
| | gtgaaccagc gcggcgagct gtgcgtccgt ggccccatga tcatgagcgg ctacgttaac | |
| | aaccccgagg ctacaaacgc | |
| | tctcatcgac aaggacggct ggctgcacag cggcgacatc gcctactggg | |
| | acgaggacga gcacttcttc atcgtggacc ggctgaagag cctgatcaaa tacaagggct | |
| | accaggtggc cccagccgaa ctggagagca tcctgctgca acaccccaac atcttcgacg | |
| | ccggggtcgc cggcctgccc gacgacgatg ccggcgagct gcccgccgca | |
| | gtcgtcgtgc tggaacacgg caaaaccatg accgagaagg agatcgtgga ctatgtggcc | |
| | agccaggtca caaccgccaa | |
| | gaagctgcgc ggtggtgttg tgttcgtgga cgaggtgcct aaaggactga ccggcaagtt | |
| | ggacgcccgc aagatccgcg agattctcat taaggccaag aagggcggca | |
| | agatcgccgt gggcagcgga gagggcagag gaagtcttct aacatgcggt | |
| | gacgtggagg agaatcccgg ccctatggtg tccaagggcg aagaactgtt taccggcgtg | |
| | gtgcccatcc tggtggaact ggatggggat gtgaacggcc acaagttcag cgttagcgga | |
| | gaaggcgaag gcgacgccac | |
| | atacggaaag ctgaccctga agttcatctg caccaccggc aagctgcctg tgccttggcc | |
| | tacactggtc accacactga catacggcgt gcagtgcttc agcagatacc ccgaccatat | |
| | gaagcagcac gacttcttca agagcgccat gcctgagggc tacgtgcaag agcggaccat | |
| | cttctttaaa gacgacggca actacaagac cagggccgaa gtgaagttcg agggcgacac | |
| | cctggtcaac cggatcgagc tgaagggcat cgacttcaaa gaggacggca acatcctggg | |
| | ccacaagctt gagtacaact acaacagcca caacgtgtac atcatggccg acaagcagaa | |
| | aaacggcatc aaagtgaact tcaagatccg gcacaacatc gaggacggct ctgtgcagct | |
| | ggccgatcac taccagcaga acacacccat cggagatggc cctgtgctgc tgcccgataa | |
| | ccactacctg agcacccaga gcaagctgag caaggacccc aacgagaagc | |
| | gggaccacat ggtgctgctg gaatttgtga cagccgccgg aatcaccctc ggcatggatg | |
| | agctgtacaa | |
| | gtgactcgag cctgggaaca cgtgtgcaca tgcgcactct catatgccac cccatgtgcc | |
| | tttagtccac ggacccccag agcaccccca agcctgggct tgagctgcag aatgactcca | |
| | ccttctcacc tgctccagga ggtttgcagg gagctcaagc ccttggggag ggggatgcct | |
| | tcatgggggt gaccccacga tttgtcttat cccccccagc ctggccccgg cctttatgtt | |
| | ttttgtaaga taaaccgttt ttaacacata gcgccgtgct gtaaataagc ccagtgctgc | |
| | tgtaaataca ggaagaaaga gcttgaggtg ggagcggggc tgggaggaag | |
| | ggatgggccc | |
| | cgccttcctg ggcagccttt ccagcctcct gctggctctc tcttcctacc ctccttccac | |
| | atgtacataa actgtcactc taggaagaag acaaatgaca gattctgaca tttatatttg | |
| | tgtattttcc tggatttata gtatgtgact tttctgatta atatatttaa tatattgaat | |
| | aaaaaataga catgtagttg gaactgagat tcagtctgtc tctgatgccc cctccccact | |
| | cccccaccag acacacccca tcattacata agagatgggc tgctcaagat gaaacttgga | |
| | tgttaccagc ctgagctgtc aggcctcagt gtactcattt gtaaaaggcg gataataatg | |
| | acacctgctt cacgaggttg ttatgcaaag cacttagact aatttctaac acgtgggaag | |
| | cctgcattag ctgtgcctgg ctagctgtgc ctggctcatt gctggggtct gcagtggctg | |
| | actagcccag gggtcactgc agggccctag caatagactt agccgcagat ctcagggttg | |
| | tcatgtttcc taaactggac atatattctc tgattcttga tttccacatc cataaaacaa | |
| | gaatagaccc agcctcacag agctgcggcc gccaccgcgg tggagctcca gcttttgttc | |
| | cctttagtga gggttaattg cgcgcttggc gtaatcatgg tcatagctgt ttcctgtgtg | |
| | aaattgttat ccgctcacaa ttccacacaa catacgagcc ggaagcataa agtgtaaagc | |
| | ctggggtgcc taatgagtga gctaactcac attaattgcg ttgcgctcac tgcccgcttt | |
| | ccagtcggga aacctgtcgt gccagctgca ttaatgaatc ggccaacgcg cggggagagg | |
| | cggtttgcgt attgggcgct cttccgcttc ctcgctcact gactcgctgc gctcggtcgt | |
| | tcggctgcgg cgagcggtat cagctcactc aaaggcggta atacggttat ccacagaatc | |
| | aggggataac gcaggaaaga acatgtgagc aaaaggccag caaaaggcca | |
| | ggaaccgtaa aaaggccgcg ttgctggcgt ttttccatag gctccgcccc cctgacgagc | |
| | atcacaaaaa tcgacgctca agtcagaggt ggcgaaaccc gacaggacta taaagatacc | |
| | aggcgtttcc ccctggaagc tccctcgtgc gctctcctgt tccgaccctg ccgcttaccg | |
| | gatacctgtc cgcctttctc ccttcgggaa gcgtggcgct ttctcatagc tcacgctgta | |
| | ggtatctcag ttcggtgtag gtcgttcgct ccaagctggg ctgtgtgcac gaaccccccg | |
| | ttcagcccga ccgctgcgcc ttatccggta actatcgtct tgagtccaac ccggtaagac | |
| | acgacttatc gccactggca gcagccactg gtaacaggat tagcagagcg aggtatgtag | |
| | gcggtgctac | |
| | agagttcttg aagtggtggc ctaactacgg ctacactaga agaacagtat ttggtatctg | |
| | cgctctgctg aagccagtta ccttcggaaa aagagttggt agctcttgat ccggcaaaca | |
| | aaccaccgct ggtagcggtg gtttttttgt ttgcaagcag cagattacgc gcagaaaaaa | |
| | aggatctcaa gaagatcctt tgatcttttc tacggggtct gacgctcagt ggaacgaaaa | |
| | ctcacgttaa gggattttgg tcatgagatt atcaaaaagg atcttcacct agatcctttt | |
| | aaattaaaaa tgaagtttta aatcaatcta aagtatatat gagtaaactt ggtctgacag | |
| | ttaccaatgc ttaatcagtg aggcacctat ctcagcgatc tgtctatttc gttcatccat | |
| | agttgcctga ctccccgtcg tgtagataac tacgatacgg gagggcttac catctggccc | |
| | cagtgctgca atgataccgc gagacccacg ctcaccggct ccagatttat cagcaataaa | |
| | ccagccagcc ggaagggccg agcgcagaag tggtcctgca actttatccg cctccatcca | |
| | gtctattaat tgttgccggg aagctagagt aagtagttcg ccagttaata gtttgcgcaa | |
| | cgttgttgcc attgctacag gcatcgtggt gtcacgctcg tcgtttggta tggcttcatt | |
| | cagctccggt tcccaacgat caaggcgagt tacatgatcc cccatgttgt gcaaaaaagc | |
| | ggttagctcc ttcggtcctc cgatcgttgt cagaagtaag ttggccgcag tgttatcact | |
| | catggttatg gcagcactgc ataattctct tactgtcatg ccatccgtaa gatgcttttc | |
| | tgtgactggt gagtactcaa ccaagtcatt ctgagaatag tgtatgcggc gaccgagttg | |
| | ctcttgcccg gcgtcaatac gggataatac cgcgccacat agcagaactt taaaagtgct | |
| | catcattgga aaacgttctt cggggcgaaa actctcaagg atcttaccgc tgttgagatc | |
| | cagttcgatg taacccactc gtgcacccaa ctgatcttca gcatctttta ctttcaccag | |
| | cgtttctggg tgagcaaaaa caggaaggca aaatgccgca aaaaagggaa | |
| | taagggcgac | |
| | acggaaatgt tgaatactca tactcttcct ttttcaatat tattgaagca tttatcaggg | |
| | ttattgtctc atgagcggat acatatttga atgtatttag aaaaataaac aaataggggt | |
| | tccgcgcaca tttccccgaa aagtgc | |
| 6 | gaag gcagaggctc tctcctcaca tgtggggatg ttgaagaaaa tccaggtccc | T2A DNA |
| 7 | EGRGSLLTCGDVEENPGP | T2A protein |
| 8 | atggaagatg ccaaaaacat taagaagggc ccagcgccat tctacccact cgaagacggg | FFLuc2 DNA |
| | accgccggcg agcagctgca caaagccatg aagcgctacg ccctggtgcc | |
| | cggcaccatc gcctttaccg acgcacatat cgaggtggac attacctacg ccgagtactt | |
| | cgagatgagc gttcggctgg cagaagctat gaagcgctat gggctgaata caaaccatcg | |
| | gatcgtggtg tgcagcgaga atagcttgca gttcttcatg cccgtgttgg gtgccctgtt | |
| | catcggtgtg gctgtggccc cagctaacga catctacaac gagcgcgagc tgctgaacag | |
| | catgggcatc | |
| | agccagccca ccgtcgtatt cgtgagcaag aaagggctgc aaaagatcct caacgtgcaa | |
| | aagaagctac cgatcataca aaagatcatc atcatggata gcaagaccga ctaccagggc | |
| | ttccaaagca tgtacacctt cgtgacttcc catttgccac ccggcttcaa cgagtacgac | |
| | ttcgtgcccg agagcttcga ccgggacaaa accatcgccc tgatcatgaa cagtagtggc | |
| | agtaccggat tgcccaaggg cgtagcccta ccgcaccgca ccgcttgtgt ccgattcagt | |
| | catgcccgcg accccatctt cggcaaccag atcatccccg acaccgctat cctcagcgtg | |
| | gtgccatttc accacggctt cggcatgttc accacgctgg gctacttgat ctgcggcttt | |
| | cgggtcgtgc tcatgtaccg cttcgaggag gagctattct tgcgcagctt gcaagactat | |
| | aagattcaat ctgccctgct ggtgcccaca ctatttagct tcttcgctaa gagcactctc | |
| | atcgacaaat acgacctaag caacttgcac gagatcgcca gcggcggggc | |
| | gccgctcagc aaggaagtcg gcgaggccgt ggccaaacgc ttccacctac | |
| | ccggcatccg ccagggctac ggcctgacag aaacaaccag cgccattctg atcacccccg | |
| | aaggggacga caagcctggc gcagtaggca aggtggtgcc cttcttcgag gctaaggtgg | |
| | tggacttgga caccggcaag | |
| | acactgggtg tgaaccagcg cggcgagctg tgcgtccgtg gccccatgat catgagcggc | |
| | tacgttaaca accccgaggc tacaaacgct ctcatcgaca aggacggctg gctgcacagc | |
| | ggcgacatcg cctactggga cgaggacgag cacttcttca tcgtggaccg gctgaagagc | |
| | ctgatcaaat acaagggcta ccaggtggcc ccagccgaac tggagagcat cctgctgcaa | |
| | caccccaaca tcttcgacgc cggggtcgcc ggcctgcccg acgacgatgc | |
| | cggcgagctg | |
| | cccgccgcag tcgtcgtgct ggaacacggc aaaaccatga ccgagaagga | |
| | gatcgtggac tatgtggcca gccaggtcac aaccgccaag aagctgcgcg gtggtgttgt | |
| | gttcgtggac gaggtgccta aaggactgac cggcaagttg gacgcccgca agatccgcga | |
| | gattctcatt aaggccaaga agggcggcaa gatcgccgtg | |
| 9 | MEDAKNIKKG PAPFYPLEDG TAGEQLHKAM KRYALVPGTI | FFLuc2 Protein |
| | AFTDAHIEVD ITYAEYFEMS VRLAEAMKRY GLNTNHRIVV | |
| | CSENSLQFFM PVLGALFIGV AVAPANDIYN ERELLNSMGI | |
| | SQPTVVFVSK KGLQKILNVQ KKLPIIQKII IMDSKTDYQG | |
| | FQSMYTFVTS HLPPGFNEYD FVPESFDRDK TIALIMNSSG | |
| | STGLPKGVAL PHRTACVRFS HARDPIFGNQ IIPDTAILSV | |
| | VPFHHGFGMF TTLGYLICGF RVVLMYRFEE ELFLRSLQDY | |
| | KIQSALLVPT LFSFFAKSTL IDKYDLSNLH EIASGGAPLS | |
| | KEVGEAVAKR FHLPGIRQGY GLTETTSAIL ITPEGDDKPG | |
| | AVGKVVPFFE AKVVDLDTGK TLGVNQRGEL | |
| | CVRGPMIMSG YVNNPEATNA LIDKDGWLHS | |
| | GDIAYWDEDE HFFIVDRLKS LIKYKGYQVA PAELESILLQ | |
| | HPNIFDAGVA GLPDDDAGEL PAAVVVLEHG KTMTEKEIVD | |
| | YVASQVTTAK KLRGGVVFVD EVPKGLTGKL DARKIREILI | |
| | KAKKGGKIAV | |
| 10 | atggtgtcca agggcgaaga actgtttacc ggcgtggtgc ccatcctggt ggaactggat | eGFP DNA |
| | ggggatgtga acggccacaa gttcagcgtt agcggagaag gcgaaggcga | |
| | cgccacatac ggaaagctga ccctgaagtt catctgcacc accggcaagc tgcctgtgcc | |
| | ttggcctaca ctggtcacca cactgacata cggcgtgcag tgcttcagca gataccccga | |
| | ccatatgaag cagcacgact tcttcaagag cgccatgcct gagggctacg tgcaagagcg | |
| | gaccatcttc | |
| | tttaaagacg acggcaacta caagaccagg gccgaagtga agttcgaggg cgacaccctg | |
| | gtcaaccgga tcgagctgaa gggcatcgac ttcaaagagg acggcaacat cctgggccac | |
| | aagcttgagt acaactacaa cagccacaac gtgtacatca tggccgacaa gcagaaaaac | |
| | ggcatcaaag tgaacttcaa gatccggcac aacatcgagg acggctctgt gcagctggcc | |
| | gatcactacc agcagaacac acccatcgga gatggccctg tgctgctgcc cgataaccac | |
| | tacctgagca cccagagcaa gctgagcaag gaccccaacg agaagcggga | |
| | ccacatggtg ctgctggaat ttgtgacagc cgccggaatc accctcggca tggatgagct | |
| | gtacaag | |
| 11 | MVSKGEELFT GVVPILVELD GDVNGHKFSV SGEGEGDATY | eGFP Protein |
| | GKLTLKFICT TGKLPVPWPT | |
| | LVTTLTYGVQ CFSRYPDHMK QHDFFKSAMP EGYVQERTIF | |
| | FKDDGNYKTR AEVKFEGDTL VNRIELKGID FKEDGNILGH | |
| | KLEYNYNSHN VYIMADKQKN GIKVNFKIRH NIEDGSVQLA | |
| | DHYQQNTPIG DGPVLLPDNH YLSTQSKLSK DPNEKRDHMV | |
| | LLEFVTAAGI TLGMDELYK | |
| 12 | AATCTCACTATGTTGCCCGAGCTGGTCTCGAACTCCTGG | Nur77 left |
| | GCTCAAATGATCCTCCTGTCTCAGCCTCCTAAAGTGC | homology arm |
| | TGGGATTACAGGTGTGAGCCACCACGCCTAGCCCTTCA | (chr12: |
| | CTGTGACTTCTGACAGTGCAGATCAGATTGGTTGTGCC | 52,058,015- |
| | TGTTTTGGACTTTATGTAAATGTAGTTCTGCAGGAT | 52,058,941 |
| | GGAATCTGGTGTTGAATGCAGAGGTTTTCAGATTTCT | hg38 |
| | CTGTTTTTTAAAGGAAAGAATCCACCCTCGTTCATTT | assembly), |
| | TTTCACTTAAATTGCACAGGGGACCCAACGATATAGA | with silent |
| | ACACAATCAGAGGTACTCTGGGCTGAGGGAGTGCTGA | mutations |
| | GTTCTGAGGCTGGGTTTCTCAGAACAGTCTAGATTTT | |
| | AAAAACCCAATGATCTAGCCAGAAAACGTAGGTTAGG | |
| | ATTTTATTTCCCGTTTGTGACCCTGGGCAAGTCATTAGC | |
| | CTCCTGGGCCTCGGGTTCTCACTTGGAGTATGAGGATA | |
| | ATGAGGGTTACTGCTTCTCAGACTTGTGACGATGCTTA | |
| | CTAATGGCCAACATGTGAATGCGCTTTTGTGAAGTG | |
| | CCAGCAGAGCATGAGGGGTGGTCAGGGGCAGCAGT | |
| | TTTAGGGGCCTGGGGGAGGCTGGGGCTTTGGGGGC | |
| | CTGGTTCTCAGATGTACAGCTAATCCTGTACCCTTC | |
| | CCGCAGACCGGCATGGGCTGCAGGAGCCGCGGC | |
| | GGGTGGAGGAGCTGCAGAACCGCATCGCCAGCT | |
| | GCCTGAAGGAGCACGTGGCAGCTGTGGCGGGCG | |
| | AGCCCCAGCCAGCCAGCTGCCTGTCACGTCTGTTG | |
| | GGCAAACTGCCCGAGCTGCGGACCCTGTGCACC | |
| | CAGGGCCTGCAGCGTATCTTCTACCTCAAGCTGG | |
| | AGGACTTGGTGCCCCCTCCACCtATCATCGA | |
| | CAAGATCTTCATGGACACGCTGCCCTTC | |
| 13 | GCCTGGGAACACGTGTGCACATGCGCACTCTCATATG | Nur77 right |
| | CCACCCCATGTGCCTTTAGTCCACGGACCCCCAGAGC | homology arm |
| | ACCCCCAAGCCTGGGCTTGAGCTGCAGAATGACTCC | (chr12: |
| | ACCTTCTCACCTGCTCCAGGAGGTTTGCAGGGAGCTC | 52,058,950- |
| | AAGCCCTTGGGGAGGGGGATGCCTTCATGGGGGTGA | 52,059,924 |
| | CCCCACGATTTGTCTTATCCCCCCCAGCCTGGCCCCG | hg38 |
| | GCCTTTATGTTTTTTGTAAGATAAACCGTTTTTAACA | assembly): |
| | CATAGCGCCGTGCTGTAAATAAGCCCAGTGCTGCTGT | |
| | AAATACAGGAAGAAAGAGCTTGAGGTGGGAGCGGG | |
| | GCTGGGAGGAAGGGATGGGCCCCGCCTTCCTGGGCA | |
| | GCCTTTCCAGCCTCCTGCTGGCTCTCTCTTCCTACCC | |
| | TCCTTCCACATGTACATAAACTGTCACTCTAGGAAG | |
| | AAGACAAATGACAGATTCTGACATTTATATTTGTGT | |
| | ATTTTCCTGGATTTATAGTATGTGACTTTTCTGATTA | |
| | ATATATTTAATATATTGAATAAAAAATAGACATGTA | |
| | GTTGGAACTGAGATTCAGTCTGTCTCTGATGCCCCCT | |
| | CCCCACTCCCCCACCAGACACACCCCATCATTACATA | |
| | AGAGATGGGCTGCTCAAGATGAAACTTGGATGTTAC | |
| | CAGCCTGAGCTGTCAGGCCTCAGTGTACTCATTTGTA | |
| | AAAGGCGGATAATAATGACACCTGCTTCACGAGGTT | |
| | GTTATGCAAAGCACTTAGACTAATTTCTAACACGTGG | |
| | GAAGCCTGCATTAGCTGTGCCTGGCTAGCTGTGCCTG | |
| | GCTCATTGCTGGGGTCTGCAGTGGCTGACTAGCCCAG | |
| | GGGTCACTGCAGGGCCCTAGCAATAGACTTAGCCGCA | |
| | GATCTCAGGGTTGTCATGTTTCCTAAACTGGACATATA | |
| | TTCTCTGATTCTTGATTTCCACATCCATAAAACAAGAA | |
| | TAGACCCAGCCTCACAGAGCT | |
| 14 | TCATTGACAAGATCTTCATG | Nur77 Target |
| | | Site 1 |
| 15 | GCCTGGGAACACGTGTGCA | Nur77 Target |
| | | Site 2 |
| 16 | RASQDISKYLN | CDR L1 |
| 17 | SRLHSGV | CDR L2 |
| 18 | GNTLPYTFG | CDR L3 |
| 19 | DYGVS | CDR H1 |
| 20 | VIWGSETTYYNSALKS | CDR H2 |
| 21 | YAMDYWG | CDR H3 |
| 22 | EVKLQESGPGLVAPSQSLSVTCTVSGVSLPDYGVSWIRQPPR | VH |
| | KGLEWLGVIWGSETTYYNSALKSRLTIIKDNSKSQVFLKMNS | |
| | LQTDDTAIYYCAKHYYYGGSYAMDYWGQGTSVTVSS | |
| 23 | DIQMTQTTSSLSASLGDRVTISCRASQDISKYLNWYQQKPDG | VL |
| | TVKLLIYHTSRLHSGVPSRFSGSGSGTDYSLTISNLEQEDIATY | |
| | FCQQGNTLPYTFGGGTKLEIT | |
| 24 | DIQMTQTTSSLSASLGDRVTISCRASQDISKYLNWYQQKPDG | scFv |
| | TVKLLIYHTSRLHSGVPSRFSGSGSGTDYSLTISNLEQEDIATY | |
| | FCQQGNTLPYTFGGGTKLEITGSTSGSGKPGSGEGSTKGEVK | |
| | LQESGPGLVAPSQSLSVTCTVSGVSLPDYGVSWIRQPPRKGL | |
| | EWLGVIWGSETTYYNSALKSRLTIIKDNSKSQVFLKMNSLQT | |
| | DDTAIYYCAKHYYYGGSYAMDYWGQGTSVTVSS | |
| 25 | KASQNVGTNVA | CDR L1 |
| 26 | SATYRNS | CDR L2 |
| 27 | QQYNRYPYT | CDR L3 |
| 28 | SYWMN | CDR H1 |
| 29 | QIYPGDGDTNYNGKFKG | CDR H2 |
| 30 | KTISSVVDFYFDY | CDR H3 |
| 31 | EVKLQQSGAELVRPGSSVKISCKASGYAFSSYWMNWVKQRP | VH |
| | GQGLEWIGQIYPGDGDTNYNGKFKGQATLTADKSSSTAYM | |
| | QLSGLTSEDSAVYFCARKTISSVVDFYFDYWGQGTTVTVSS | |
| 32 | DIELTQSPKFMSTSVGDRVSVTCKASQNVGTNVAWYQQKPG | VL |
| | QSPKPLIYSATYRNSGVPDRFTGSGSGTDFTLTITNVQSKDLA | |
| | DYFCQQYNRYPYTSGGGTKLEIKR | |
| 33 | GGGGSGGGGSGGGGS | Linker |
| 34 | EVKLQQSGAELVRPGSSVKISCKASGYAFSSYWMNWVKQRP | scFv |
| | GQGLEWIGQIYPGDGDTNYNGKFKGQATLTADKSSSTAYM | |
| | QLSGLTSEDSAVYFCARKTISSVVDFYFDYWGQGTTVTVSSG | |
| | GGGSGGGGSGGGGSDIELTQSPKFMSTSVGDRVSVTCKASQ | |
| | NVGTNVAWYQQKPGQSPKPLIYSATYRNSGVPDRFTGSGSG | |
| | TDFTLTITNVQSKDLADYFCQQYNRYPYTSGGGTKLEIKR | |
| 35 | HYYYGGSYAMDY | HC-CDR3 |
| 36 | HTSRLHS | LC-CDR2 |
| 37 | QQGNTLPYT | LC-CDR3 |
| 38 | gacatccagatgacccagaccacctccagcctgagcgccagcctgggcgaccgggtgaccat | Sequence |
| | cagctgccgggccagccaggacatcagcaagtacctgaactggtatcagcagaagcccgacg | encoding scFv |
| | gcaccgtcaagctgctgatctaccacaccagccggctgcacagcggcgtgcccagccggttta | |
| | gcggcagcggctccggcaccgactacagcctgaccatctccaacctggaacaggaagatatcg | |
| | ccacctacttttgccagcagggcaacacactgccctacacctttggcggcggaacaaagctgga | |
| | aatcaccggcagcacctccggcagcggcaagcctggcagcggcgagggcagcaccaaggg | |
| | cgaggtgaagctgcaggaaagcggccctggcctggtggcccccagccagagcctgagcgtg | |
| | acctgcaccgtgagcggcgtgagcctgcccgactacggcgtgagctggatccggcagccccc | |
| | caggaagggcctggaatggctgggcgtgatctggggcagcgagaccacctactacaacagcg | |
| | ccctgaagagccggctgaccatcatcaaggacaacagcaagagccaggtgttcctgaagatga | |
| | acagcctgcagaccgacgacaccgccatctactactgcgccaagcactactactacggcggca | |
| | gctacgccatggactactggggccagggcaccagcgtgaccgtgagcagc | |
| 39 | GSTSGSGKPGSGEGSTKG | Linker |
| 40 | ESKYGPPCPPCP | spacer |
| | | (IgG4hinge) |
| | | (aa) |
| 41 | GAATCTAAGTACGGACCGCCCTGCCCCCCTTGCCCT | spacer |
| | | (IgG4hinge) |
| | | (nt) |
| 42 | ESKYGPPCPPCPGQPREPQVYTLPPSQEEMTKNQVSLTCLVK | Hinge-CH3 |
| | GFYPSDIAVEWESNGQPENNYKTTPPVLDSDGSFFLYSRLTV | spacer |
| | DKSRWQEGNVFSCSVMHEALHNHYTQKSLSLSLGK | |
| 43 | ESKYGPPCPPCPAPEFLGGPSVFLFPPKPKDTLMISRTPEVTCV | Hinge-CH2-CH3 |
| | VVDVSQEDPEVQFNWYVDGVEVHNAKTKPREEQFNSTYRV | spacer |
| | VSVLTVLHQDWLNGKEYKCKVSNKGLPSSIEKTISKAKGQP | |
| | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLGK | |
| 44 | RWPESPKAQASSVPTAQPQAEGSLAKATTAPATTRNTGRGG | IgD-hinge-Fc |
| | EEKKKEKEKEEQEERETKTPECPSHTQPLGVYLLTPAVQDL | |
| | WLRDKATFTCFVVGSDLKDAHLTWEVAGKVPTGGVEEGLL | |
| | ERHSNGSQSQHSRLTLPRSLWNAGTSVTCTLNHPSLPPQRLM | |
| | ALREPAAQAPVKLSLNLLASSDPPEAASWLLCEVSGFSPPNIL | |
| | LMWLEDQREVNTSGFAPARPPPQPGSTTFWAWSVLRVPAPP | |
| | SPQPATYTCVVSHEDSRTLLNASRSLEVSYVTDH | |
| 45 | FWVLVVVGGVLACYSLLVTVAFIIFWV | CD28 (amino |
| | | acids 153-179 |
| | | of Accession |
| | | No. P10747) |
| 46 | IEVMYPPPYLDNEKSNGTIIHVKGKHLCPSPLFPGPSKP | CD28 (amino |
| | FWVLVVVGGVLACYSLLVTVAFIIFWV | acids 114-179 |
| | | of Accession |
| | | No. P10747) |
| 47 | RSKRSRLLHSDYMNMTPRRPGPTRKHYQPYAPPRDFAAYRS | CD28 (amino |
| | | acids 180-220 |
| | | of P10747) |
| 48 | RSKRSRGGHSDYMNMTPRRPGPTRKHYQPYAPPRDFAAYRS | CD28 (LL to |
| | | GG) |
| 49 | KRGRKKLLYIFKQPFMRPVQTTQEEDGCSCRFPEEEEGGCEL | 4-1BB (amino |
| | | acids 214-255 |
| | | of Q07011.1) |
| 50 | RVKFSRSADAPAYQQGQNQLYNELNLGRREEYDVLDKRRG | CD3 zeta |
| | RDPEMGGKPRRKNPQEGLYNELQKDKMAEAYSEIGMKGER | |
| | RRGKGHDGLYQGLSTATKDTYDALHMQALPPR | |
| 51 | RVKFSRSAEPPAYQQGQNQLYNELNLGRREEYDVLDKRRGR | CD3 zeta |
| | DPEMGGKPRRKNPQEGLYNELQKDKMAEAYSEIGMKGERR | |
| | RGKGHDGLYQGLSTATKDTYDALHMQALPPR | |
| 52 | RVKFSRSADAPAYKQGQNQLYNELNLGRREEYDVLDKRRG | CD3 zeta |
| | RDPEMGGKPRRKNPQEGLYNELQKDKMAEAYSEIGMKGER | |
| | RRGKGHDGLYQGLSTATKDTYDALHMQALPPR | |
| 53 | LEGGGEGRGSLLTCGDVEENPGPR | T2A |
| 54 | MLLLVTSLLLCELPHPAFLLIPRKVCNGIGIGEFKDSLSINATN | tEGFR |
| | IKHFKNCTSISGDLHILPVAFRGDSFTHTPPLDPQELDILKTVK | |
| | EITGFLLIQAWPENRTDLHAFENLEIIRGRTKQHGQFSLAVVS | |
| | LNITSLGLRSLKEISDGDVIISGNKNLCYANTINWKKLFGTSG | |
| | QKTKIISNRGENSCKATGQVCHALCSPEGCWGPEPRDCVSCR | |
| | NVSRGRECVDKCNLLEGEPREFVENSECIQCHPECLPQAMNI | |
| | TCTGRGPDNCIQCAHYIDGPHCVKTCPAGVMGENNTLVWK | |
| | YADAGHVCHLCHPNCTYGCTGPGLEGCPTNGPKIPSIATGM | |
| | VGALLLLLVVALGIGLFM | |

1. A method for determining potency of viral vectors, comprising:
a) introducing a titrated amount of a test viral vector encoding a recombinant receptor into a plurality of populations of reporter T cells, wherein each population of reporter T cells is the same and each is introduced with a different amount of the titrated test viral vector, wherein:
each of the reporter T cell populations comprise reporter T cells comprising a nucleic acid sequence encoding a reporter molecule operably linked to a transcriptional regulatory element of a T cell transcription factor;
the recombinant receptor comprises an extracellular binding domain specific to a target, a transmembrane domain and comprises or is complexed with an intracellular signaling region comprising an ITAM-containing domain;
b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule;
c) measuring each of the plurality of populations of reporter T cells for the detectable signal from the reporter molecule; and
d) determining, based on the measured detectable signal, the titrated amount of the test viral vector that results in a half-maximal detectable signal.

2. The method of claim 1, wherein the potency is a relative potency and the method further comprises comparing the half-maximal detectable signal of the test viral vector to a half-maximal detectable signal of a reference viral vector standard in the same assay.

3. A method for determining potency of viral vectors, comprising:
a) introducing a titrated amount of a test viral vector encoding a recombinant receptor into a plurality of populations of reporter T cells, wherein each population of reporter T cells is the same and each is introduced with a different amount of the titrated test viral vector, wherein:
each of the reporter T cell populations comprise reporter T cells comprising a nucleic acid sequence encoding a reporter molecule operably linked to a transcriptional regulatory element of a T cell transcription factor;
the recombinant receptor comprises an extracellular binding domain specific to a target, a transmembrane domain and an intracellular signaling region comprising an ITAM-containing domain;
b) incubating each of the plurality of populations of reporter T cells in the presence of a recombinant receptor stimulating agent, wherein binding of the recombinant receptor stimulating agent to the recombinant receptor induces signaling through the intracellular signaling region of the recombinant receptor to produce a detectable signal from the reporter molecule;
c) measuring each of the plurality of populations of reporter T cells for the detectable signal from the reporter molecule; and
d) determining, based on the measured detectable signal, the relative potency of the viral test viral vector by comparing the half-maximal detectable signal to a half-maximal detectable signal of a reference viral vector standard in the same assay.

4. The method of claim 2, wherein the relative potency is a percentage of the detectable signal of the test viral vector to the reference viral vector standard, or wherein the relative potency is a ratio of the detectable signal of the test viral vector to the reference viral vector standard.

5. (canceled)

6. The method of claim 1, wherein the titrated amount of a test viral vector is a serial dilution of the viral vector.

7. The method claim 6, wherein the serial dilution of the viral vector is a serial dilution based on the vector volume, the serial dilution is a serial dilution based on the viral vector titer, or wherein the serial dilution is a serial dilution based on the multiplicity of infection (MOI) of the viral vector.

8. (canceled)

9. The method of claim 7, wherein the viral vector titer is a functional titer, optionally wherein the functional titer is quantified by in vitro plaque assay, or wherein the viral vector titer is a physical titer, optionally wherein the physical titer is quantified via DNA or RNA quantification by a PCR method.

10-13. (canceled)

14. The method of claim 1, wherein the titrated amount of a test viral vector is a ratio of a constant amount of viral vector to the number of cells in the population of reporter T cells.

15. The method of claim 14, wherein the amount of the test viral vector is a volume of the test viral vector, the amount of test viral vector is a titer of the test viral vector, or wherein the amount of the test viral vector is a MOI of a test viral vector.

16-19. (canceled)

20. The method of claim 1, where in the reporter T cell is a Jurkat cell line or a derivative thereof.

21. (canceled)

22. The method of claim 1, wherein the transcriptional regulatory element comprises a response element or elements recognized by the transcription factor that is activated upon signaling through the ITAM-containing domain of the recombinant receptor induced by the recombinant receptor stimulating agent.

23. The method of claim 1, wherein the transcription factor is selected from the group consisting of Nur77, NF-κB, NFAT or AP1.

24. (canceled)

25. The method of claim 22, wherein the transcriptional regulatory element comprises the Nur77 promoter or portion thereof containing a response element or elements recognized by a transcription factor.

26. The method of claim 25, wherein the transcriptional regulatory element is a transcriptional regulatory element within an endogenous Nur77 locus in the T cell.

27. The method of claim 26, wherein the nucleic acid sequence encoding the reporter molecule is integrated in the genome of the reporter T cell at or near the endogenous locus encoding Nur77, wherein the reporter molecule is operably linked to a transcriptional regulatory element of the endogenous Nur77 locus.

28. The method of claim 27, wherein the nucleic acid sequence encoding the reporter molecule is integrated by:
a) inducing a genetic disruption at one or more target site(s) at or near the endogenous locus encoding Nur77; and
b) introducing a template polynucleotide comprising a nucleic acid encoding the reporter molecule for knock-in of the reporter molecule in the endogenous locus by homology directed repair (HDR).

29. (canceled)

30. (canceled)

31. The method of claim 28, wherein the nucleic acid encoding the reporter is present within the genome at a site that is at or near the final exon of the endogenous locus encoding Nur77.

32. The method of claim 31, wherein the nucleic acid is present within the genome at a site comprising, the nucleic acid sequence TCATTGACAAGATCTTCATG (SEQ ID NO:3) and/or GCCTGGGAACACGTGTGCA (SEQ ID NO:4).

33. (canceled)

34. The method of claim 1, wherein the reporter molecule is a luciferase, optionally firefly luciferase.

35. The method of claim 1, wherein the nucleic acid sequence encoding the reporter molecule further encodes one or more marker(s) that is or comprises a transduction marker and/or a selection marker.

36. The method of claim 35, wherein the transduction marker comprises a fluorescent protein, optionally eGFP.

37-40. (canceled)

41. The method of claim 1, wherein the intracellular signaling domain is or comprises a CD3-zeta (CD3) chain or a signaling portion thereof.

42. The method of claim 1, wherein the intracellular signaling region further comprises a costimulatory signaling region.

43. (canceled)

44. (canceled)

45. The method of claim 1, wherein the recombinant receptor is an engineered T cell receptor (eTCR) or is a chimeric antigen receptor (CAR).

46. (canceled)

47. The method of claim 1, wherein the recombinant receptor stimulating agent comprises a binding molecule that is or comprises a target antigen or an extracellular domain binding portion thereof of the recombinant receptor, or wherein the recombinant receptor stimulating agent comprises a binding molecule that is an antibody specific to an extracellular domain of the recombinant receptor.

48. (canceled)

49. (canceled)

50. The method of claim 1, wherein the recombinant receptor stimulating agent is immobilized or attached to a solid support.

51-56. (canceled)

57. The method of claim 1, wherein the recombinant receptor stimulating agent is a target-expressing cell from a cell line, a primary cell taken from a subject, or a cell that has been introduced to express the target of the recombinant receptor.

58-62. (canceled)

63. The method of claim 1, wherein the plurality of incubations are performed in a flask, a tube, or a multi-well plate.

64. (canceled)

65. (canceled)

66. The method of claim 1, wherein the detectable signal is measured using a plate reader.

67. (canceled)

68. The method of claim 1, wherein the virial vector is an adenoviral vector, adeno-associated viral vector, or a retroviral vector.

69-71. (canceled)

72. The method of claim 1, wherein the detectable signal is luciferase luminescence.


SEQUENCE LISTING (54 sequences)

SEQ ID NO: 1 - DNA, 2597 bp, Artificial Sequence
  Human Nur77 DNA
ttcctggtgtaagctttggtatggatggtggccgtctccctacagactgggagctgttagagggcagggatcctagctgacacatctatgtcctcgccttggttggaggcctccaccatggacagaggccaggccctgcccctcccaggcagcctggctccttctgctgggccctgaaggcagacgggataatgtggttggccaaggcctgttggtccatccagagtgagatgccctgtatccaagcccaatatgggacaccagcaccgagtccgggaccccgtgaccactggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctggctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggcggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgctcgccagctgcctgaaggagcacgtggcagctgtggcgggcgagccccacagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaactgaaaaaaaaaaaaaa

SEQ ID NO: 2 - protein, 609 aa, Artificial Sequence
  Human Nur77
MWLAKACWSIQSEMPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLP

SEQ ID NO: 3 - RNA, 20 nt, Artificial Sequence
  Human Nur77 gRNA 1
caugaagaucuugucaauga

SEQ ID NO: 4 - RNA, 19 nt, Artificial Sequence
  Human Nur77 gRNA 2
ugcacacguguucccaggc

SEQ ID NO: 5 - DNA, 7286 bp, Artificial Sequence
  Nur77 Knock-in construct
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaatctcactatgttgcccgagctggtctcgaactcctgggctcaaatgatcctcctgtctcagcctcctaaagtgctgggattacaggtgtgagccaccacgcctagcccttcactgtgacttctgacagtgcagatcagattggttgtgcctgttttggactttatgtaaatgtagttctgcaggatggaatctggtgttgaatgcagaggttttcagatttctctgttttttaaaggaaagaatccaccctcgttcattttttcacttaaattgcacaggggacccaacgatatagaacacaatcagaggtactctgggctgagggagtgctgagttctgaggctgggtttctcagaacagtctagattttaaaaacccaatgatctagccagaaaacgtaggttaggattttatttcccgtttgtgaccctgggcaagtcattagcctcctgggcctcgggttctcacttggagtatgaggataatgagggttactgcttctcagacttgtgacgatgcttactaatggccaacatgtgaatgcgcttttgtgaagtgccagcagagcatgaggggtggtcaggggcagcagttttaggggcctgggggaggctggggctttgggggcctggttctcagatgtacagctaatcctgtacccttcccgcagaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacctatcatcgacaagatcttcatggacacgctgcccttcggatccggagaaggacggggctctctgcttacatgtggcgacgttgaggaaaaccccggacctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaaatacgacctaagcaacttgcacgagatcgccagcggcggggcgccgctcagcaaggaagtcggcgaggccgtggccaaacgcttccacctacccggcatccgccagggctacggcctgacagaaacaaccagcgccattctgatcacccccgaaggggacgacaagcctggcgcagtaggcaaggtggtgcccttcttcgaggctaaggtggtggacttggacaccggcaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtggccccagccgaactggagagcatcctgctgcaacaccccaacatcttcgacgccggggtcgccggcctgcccgacgacgatgccggcgagctgcccgccgcagtcgtcgtgctggaacacggcaaaaccatgaccgagaaggagatcgtggactatgtggccagccaggtcacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggcagcggagagggcagaggaagtcttctaacatgcggtgacgtggaggagaatcccggccctatggtgtccaagggcgaagaactgtttaccggcgtggtgcccatcctggtggaactggatggggatgtgaacggccacaagttcagcgttagcggagaaggcgaaggcgacgccacatacggaaagctgaccctgaagttcatctgcaccaccggcaagctgcctgtgccttggcctacactggtcaccacactgacatacggcgtgcagtgcttcagcagataccccgaccatatgaagcagcacgacttcttcaagagcgccatgcctgagggctacgtgcaagagcggaccatcttctttaaagacgacggcaactacaagaccagggccgaagtgaagttcgagggcgacaccctggtcaaccggatcgagctgaagggcatcgacttcaaagaggacggcaacatcctgggccacaagcttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacccagagcaagctgagcaaggaccccaacgagaagcgggaccacatggtgctgctggaatttgtgacagccgccggaatcaccctcggcatggatgagctgtacaagtgactcgagcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaactgagattcagtctgtctctgatgccccctccccactcccccaccagacacaccccatcattacataagagatgggctgctcaagatgaaacttggatgttaccagcctgagctgtcaggcctcagtgtactcatttgtaaaaggcggataataatgacacctgcttcacgaggttgttatgcaaagcacttagactaatttctaacacgtgggaagcctgcattagctgtgcctggctagctgtgcctggctcattgctggggtctgcagtggctgactagcccaggggtcactgcagggccctagcaatagacttagccgcagatctcagggttgtcatgtttcctaaactggacatatattctctgattcttgatttccacatccataaaacaagaatagacccagcctcacagagctgcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 6 - DNA, 54 bp, Artificial Sequence
  T2A DNA
gaaggcagaggctctctcctcacatgtggggatgttgaagaaaatccaggtccc

SEQ ID NO: 7 - protein, 18 aa, Artificial Sequence
  T2A protein
EGRGSLLTCGDVEENPGP

SEQ ID NO: 8 - DNA, 1650 bp, Artificial Sequence
  FFLuc2 DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaaatacgacctaagcaacttgcacgagatcgccagcggcggggcgccgctcagcaaggaagtcggcgaggccgtggccaaacgcttccacctacccggcatccgccagggctacggcctgacagaaacaaccagcgccattctgatcacccccgaaggggacgacaagcctggcgcagtaggcaaggtggtgcccttcttcgaggctaaggtggtggacttggacaccggcaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtggccccagccgaactggagagcatcctgctgcaacaccccaacatcttcgacgccggggtcgccggcctgcccgacgacgatgccggcgagctgcccgccgcagtcgtcgtgctggaacacggcaaaaccatgaccgagaaggagatcgtggactatgtggccagccaggtcacaaccgccaagaagctgcgcggtggtgttgtgttcgtggacgaggtgcctaaaggactgaccggcaagttggacgcccgcaagatccgcgagattctcattaaggccaagaagggcggcaagatcgccgtg

SEQ ID NO: 9 - protein, 550 aa, Artificial Sequence
  FFLuc2
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 10 - DNA, 717 bp, Artificial Sequence
  eGFP
atggtgtccaagggcgaagaactgtttaccggcgtggtgcccatcctggtggaactggatggggatgtgaacggccacaagttcagcgttagcggagaaggcgaaggcgacgccacatacggaaagctgaccctgaagttcatctgcaccaccggcaagctgcctgtgccttggcctacactggtcaccacactgacatacggcgtgcagtgcttcagcagataccccgaccatatgaagcagcacgacttcttcaagagcgccatgcctgagggctacgtgcaagagcggaccatcttctttaaagacgacggcaactacaagaccagggccgaagtgaagttcgagggcgacaccctggtcaaccggatcgagctgaagggcatcgacttcaaagaggacggcaacatcctgggccacaagcttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacccagagcaagctgagcaaggaccccaacgagaagcgggaccacatggtgctgctggaatttgtgacagccgccggaatcaccctcggcatggatgagctgtacaag

SEQ ID NO: 11 - protein, 239 aa, Artificial Sequence
  eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 12 - DNA, 927 bp, Artificial Sequence
  Nur77 left homology arm
aatctcactatgttgcccgagctggtctcgaactcctgggctcaaatgatcctcctgtctcagcctcctaaagtgctgggattacaggtgtgagccaccacgcctagcccttcactgtgacttctgacagtgcagatcagattggttgtgcctgttttggactttatgtaaatgtagttctgcaggatggaatctggtgttgaatgcagaggttttcagatttctctgttttttaaaggaaagaatccaccctcgttcattttttcacttaaattgcacaggggacccaacgatatagaacacaatcagaggtactctgggctgagggagtgctgagttctgaggctgggtttctcagaacagtctagattttaaaaacccaatgatctagccagaaaacgtaggttaggattttatttcccgtttgtgaccctgggcaagtcattagcctcctgggcctcgggttctcacttggagtatgaggataatgagggttactgcttctcagacttgtgacgatgcttactaatggccaacatgtgaatgcgcttttgtgaagtgccagcagagcatgaggggtggtcaggggcagcagttttaggggcctgggggaggctggggctttgggggcctggttctcagatgtacagctaatcctgtacccttcccgcagaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgtatcttctacctcaagctggaggacttggtgccccctccacctatcatcgacaagatcttcatggacacgctgcccttc

SEQ ID NO: 13 - DNA, 975 bp, Artificial Sequence
  Nur77 right homology arm
gcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaactgagattcagtctgtctctgatgccccctccccactcccccaccagacacaccccatcattacataagagatgggctgctcaagatgaaacttggatgttaccagcctgagctgtcaggcctcagtgtactcatttgtaaaaggcggataataatgacacctgcttcacgaggttgttatgcaaagcacttagactaatttctaacacgtgggaagcctgcattagctgtgcctggctagctgtgcctggctcattgctggggtctgcagtggctgactagcccaggggtcactgcagggccctagcaatagacttagccgcagatctcagggttgtcatgtttcctaaactggacatatattctctgattcttgatttccacatccataaaacaagaatagacccagcctcacagagct

SEQ ID NO: 14 - DNA, 20 bp, Artificial Sequence
  Nur77 Target Site 1
tcattgacaagatcttcatg

SEQ ID NO: 15 - DNA, 19 bp, Artificial Sequence
  Nur77 Target Site 2
gcctgggaacacgtgtgca

SEQ ID NO: 16 - protein, 11 aa, Artificial Sequence
  CDR L1
RASQDISKYLN

SEQ ID NO: 17 - protein, 7 aa, Artificial Sequence
  CDR L2
SRLHSGV

SEQ ID NO: 18 - protein, 9 aa, Artificial Sequence
  CDR L3
GNTLPYTFG

SEQ ID NO: 19 - protein, 5 aa, Artificial Sequence
  CDR H1
DYGVS

SEQ ID NO: 20 - protein, 16 aa, Artificial Sequence
  CDR H2
VIWGSETTYYNSALKS

SEQ ID NO: 21 - protein, 7 aa, Artificial Sequence
  CDR H3
YAMDYWG

SEQ ID NO: 22 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 23 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 24 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 25 - protein, 11 aa, Artificial Sequence
  CDR L1
KASQNVGTNVA

SEQ ID NO: 26 - protein, 7 aa, Artificial Sequence
  CDR L2
SATYRNS

SEQ ID NO: 27 - protein, 9 aa, Artificial Sequence
  CDR L3
QQYNRYPYT

SEQ ID NO: 28 - protein, 5 aa, Artificial Sequence
  CDR H1
SYWMN

SEQ ID NO: 29 - protein, 17 aa, Artificial Sequence
  CDR H2
QIYPGDGDTNYNGKFKG

SEQ ID NO: 30 - protein, 13 aa, Artificial Sequence
  CDR H3
KTISSVVDFYFDY

SEQ ID NO: 31 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 32 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 33 - protein, 15 aa, Artificial Sequence
  Linker
GGGGSGGGGSGGGGS

SEQ ID NO: 34 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 35 - protein, 12 aa, Artificial Sequence
  HC-CDR3
HYYYGGSYAMDY

SEQ ID NO: 36 - protein, 7 aa, Artificial Sequence
  LC-CDR2
HTSRLHS

SEQ ID NO: 37 - protein, 9 aa, Artificial Sequence
  LC-CDR3
QQGNTLPYT

SEQ ID NO: 38 - DNA, 735 bp, Artificial Sequence
  scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 39 - protein, 18 aa, Artificial Sequence
  Linker
GSTSGSGKPGSGEGSTKG

SEQ ID NO: 40 - protein, 12 aa, Artificial Sequence
  spacer
ESKYGPPCPPCP

SEQ ID NO: 41 - DNA, 36 bp, Artificial Sequence
  spacer
gaatctaagtacggaccgccctgccccccttgccct

SEQ ID NO: 42 - protein, 119 aa, Artificial Sequence
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 229 aa, Artificial Sequence
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 282 aa, Artificial Sequence
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 45 - protein, 27 aa, Artificial Sequence
  CD28
FWVLVVVGGVLACYSLLVTVAFIIFWV

SEQ ID NO: 46 - protein, 66 aa, Artificial Sequence
  CD28
IEVMYPPPYLDNEKSNGTIIHVKGKHLCPSPLFPGPSKPFWVLVVVGGVLACYSLLVTVAFIIFWV

SEQ ID NO: 47 - protein, 41 aa, Artificial Sequence
  CD28
RSKRSRLLHSDYMNMTPRRPGPTRKHYQPYAPPRDFAAYRS

SEQ ID NO: 48 - protein, 41 aa, Artificial Sequence
  CD28
RSKRSRGGHSDYMNMTPRRPGPTRKHYQPYAPPRDFAAYRS

SEQ ID NO: 49 - protein, 42 aa, Artificial Sequence
  4-1BB
KRGRKKLLYIFKQPFMRPVQTTQEEDGCSCRFPEEEEGGCEL

SEQ ID NO: 50 - protein, 112 aa, Artificial Sequence
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 112 aa, Artificial Sequence
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 112 aa, Artificial Sequence
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 24 aa, Artificial Sequence
  T2A
LEGGGEGRGSLLTCGDVEENPGPR

SEQ ID NO: 54 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM